(19) United States
(12) Patent Application Publication
(10) Pub. No.: US 2024/0149115 A1
(43) Pub. Date: May 9, 2024

(54) METHOD AND SYSTEM FOR PROVIDING DIGITALLY-BASED MUSCULOSKELETAL REHABILITATION THERAPY

(71) Applicant: EVEREX, Seoul (KR)

(72) Inventors: Chan YOON, Seoul (KR); Jong Jin PARK, Seoul (KR); Chi Hyun CHOI, Hanam-si, Gyeonggi-do (KR)

(21) Appl. No.: 18/531,529

(22) Filed: Dec. 6, 2023

Related U.S. Application Data
(63) Continuation of application No. PCT/KR2023/003809, filed on Mar. 22, 2023.

(30) Foreign Application Priority Data
Nov. 3, 2022 (KR) 10-2022-0145283
Dec. 16, 2022 (KR) 10-2022-0177460

(51) Int. Cl. A63B 24/00; A63B 71/06; G16H 20/30

(52) U.S. Cl. CPC A63B 24/0075; A63B 71/0622; G16H 20/30; A63B 2024/0068; A63B 2071/0675

(57) ABSTRACT

Described herein is a method of providing digitally-based musculoskeletal rehabilitation therapy provided in an application to achieve the above-mentioned objects, the method may include: selecting, based on prescription information including an exercise plan for a patient being allocated from a doctor terminal, an exercise plan to be provided to the patient; executing the application on a user terminal to which an account of the patient is logged in; providing an exercise list according to the exercise plan to the user terminal on which the application is executed; playing, on the user terminal, an exercise image corresponding to each of a plurality of exercise items according to the plurality of exercise items constituting the exercise list; providing, based on a degree of playback of the exercise image satisfying a preset standard, an evaluation page for performing an evaluation related to the exercise item; and updating the exercise plan based on evaluation information received through the evaluation page.

### CROSS REFERENCE TO RELATED APPLICATION

The application is a Continuation Application of International Application PCT/KR2023/003809 filed on Mar. 22, 2023, which claims priority to Korean Patent Application No. 10-2022-0145283, filed on Nov. 3, 2022 and Korean Patent Application No. 10-2022-0177460, filed on Dec. 16, 2022, the entire contents of which is incorporated herein for all purposes by this reference.

### FIELD OF THE INVENTION

The present invention relates to a method and system for providing musculoskeletal rehabilitation therapy on a digital basis.

### DESCRIPTION OF THE RELATED ART

A musculoskeletal disease is pain or damage to a musculoskeletal system, such as muscles, nerves, tendons, ligaments, bones, and surrounding tissues. The musculoskeletal disease appears in many places in the body, including the neck and back, arms and legs, and more.

According to the World Health Organization (WHO), the economic losses from the musculoskeletal disease are the fourth highest of all diseases, and the musculoskeletal disease is chronic pain that affects not only daily life but also economic activity.

Meanwhile, the principle of treating musculoskeletal disease is to start with the least invasive treatment, which means that non-drug conservative treatments (e.g., exercise therapy and education, cognitive therapy, or relaxation therapy) should be performed first, and then drug and surgical treatments should be considered sequentially.

Treatment guidelines strongly recommend non-drug conservative treatment of musculoskeletal disease, and there is active research, mainly in the United States and Europe, on methods of performing non-drug conservative treatments of musculoskeletal disease.

Meanwhile, as technology advances, electronic devices (e.g., smartphones, tablet PCs, etc.) have become more popular, and accordingly, there is an increasing dependency on the Internet for many aspects of daily life.

As such, with the development of various technologies, including the Internet, behavioral patterns that were previously highly dependent on offline activities have gradually shifted to online, and currently, the activities centered around the online world have been experiencing exponential growth.

In response to this trend, even offline-based fields of industry, such as the healthcare industry, are increasingly attempting to provide healthcare services online.

In particular, in recent years, various healthcare services have been provided online, and patients, i.e., users, have been able to have medical consultations with healthcare providers about their illnesses with just a few clicks on an internet-connected electronic device.

As an example of such technology, Korean Patent No. 10-2195512 discloses a technology related to a server and system that provides an online healthcare platform and provides information on healthcare service provision points to patients online.

In response to this trend, there is a need to provide non-drug conservative treatment of musculoskeletal disease online.

### DISCLOSURE

### Technical Problem

The present invention relates to a method and system for providing a digitally-based musculoskeletal rehabilitation therapy, which is capable of providing rehabilitation therapy for a musculoskeletal disease on a digital basis.

Further, the present invention relates to a method and system for providing digitally-based musculoskeletal rehabilitation therapy that is capable of providing a personalized digital remedy suitable for an indication of a patient.

Further, the present disclosure relates to a method and system for providing digitally-based musculoskeletal rehabilitation therapy that is capable of providing cognitive behavioral therapy in conjunction with rehabilitation therapy.

There is provided a method of providing digitally-based musculoskeletal rehabilitation therapy provided in an application to achieve the above-mentioned objects, the method may include: selecting, based on prescription information including an exercise plan corresponding to an indication of a patient being allocated from a doctor terminal, an exercise plan to be provided to the patient; executing the application on a user terminal to which an account of the patient is logged in; providing an exercise list according to the exercise plan to the user terminal on which the application is executed; playing, on the user terminal, an exercise image corresponding to each of a plurality of exercise items according to the plurality of exercise items constituting the exercise list; providing, based on a degree of playback of the exercise image satisfying a preset standard, an evaluation page for performing an evaluation related to the exercise item; and updating the exercise plan based on evaluation information received through the evaluation page.

In an embodiment, the exercise plan may include exercise items related to an indication of the patient included in the prescription information on the patient, at least some of which are allocated to each of a plurality of different days constituting a preset rehabilitation period.

In an embodiment, in the providing of the exercise list, the user terminal may provide the exercise list including the plurality of exercise items allocated to specific days on which the exercise image is played, based on a reference date from which a counting of the preset rehabilitation period has been started, and in which the evaluation page may be provided to the user terminal for performing an evaluation related to the plurality of exercise items provided to the patient on each of the specific days, when a degree of playback of the exercise image satisfies a preset standard.

In an embodiment, the evaluation page may include at least one of a first evaluation area configured to evaluate exercise difficulty for the plurality of exercise items allocated to the specific day, a second evaluation area configured to select a high difficulty exercise among the plurality of exercise items, and a third evaluation area configured to evaluate exercise pain related to the plurality of exercise items, and which in the updating of the exercise plan, the difficulty of the exercise plan may be changed, or at least some of the exercise items constituting the exercise plan may be changed, based on the evaluation information received through at least one of the first evaluation area, the second evaluation area, and the third evaluation area.

In an embodiment, the exercise items according to the updated exercise plan may be provided to the user terminal, beginning on a day after the specific day has elapsed.

In an embodiment, the second evaluation area may display the plurality of exercise items allocated to the specific day, and in which, in the updating of the exercise plan, when at least one of the plurality of exercise items is selected from the user terminal, the selected exercise item may be excluded from the exercise plan and a different exercise item having an efficacy corresponding to the selected exercise item and causing less pain than the selected exercise item may be included in the exercise plan.

Further, the method may further include: allocating, to the patient account, a cognitive behavioral therapy plan that proceeds in conjunction with the exercise plan, in which the allocating of the cognitive behavioral therapy plan may include: receiving, through the user terminal, survey response data for a plurality of survey data; detecting, based on the survey response data, state information on the pain patient related to pain duration and a degree of cognitive distortion of the pain patient; specifying a user group corresponding to the state information on the pain patient among a plurality of user groups categorized according to the pain duration and degree of cognitive distortion; determining an initial therapy protocol corresponding to the user group among a plurality of therapy protocols; and providing a plurality of specific therapy programs included in the initial therapy protocol, sequentially during a preset rehabilitation period.

Further, the method may further include: providing, in response to the application being executed on the user terminal, an initial screen page, in which the initial screen page may include at least one of: a first menu item configured to access an exercise list according to the exercise plan; a second menu item configured to access the evaluation page; a third menu item configured to access the cognitive behavioral therapy plan allocated in conjunction with the exercise plan; and a fourth menu item configured to access a page for performing a functional evaluation of a specific motion of the patient, and in which, when the degree of playback of the exercise image does not satisfy the preset standard, the provision of the evaluation page to the user terminal may be restricted, even though the second menu item is selected on the user terminal.

In an embodiment, the functional evaluation of the specific motion may be configured such that the exercise plan is performed at a preset day interval during the preset rehabilitation period to which the exercise plan is allocated, and in which the fourth menu item may be configured to be included in the initial screen page on a specific day according to the preset day interval, and not to be included in the initial screen page, when not on the specific day during the rehabilitation period.

Further, there is provided a system 100 for providing digitally-based musculoskeletal rehabilitation therapy, the system may include: a communication unit configured to receive, from a doctor terminal, prescription information including an exercise plan for a patient; and a control unit, in response to an execution of an application on a user terminal to which the patient account is logged in, configured to provide an exercise list according to the exercise plan to the user terminal, in which the control unit may be configured to: play, on the user terminal, an exercise image corresponding to each of a plurality of exercise items constituting the exercise list according to the plurality of exercise items; provide, based on a degree of playback of the exercise image satisfying a preset standard, an evaluation page for performing an evaluation related to the exercise item; and update, based on evaluation information received through the evaluation page, the exercise plan.

Further, there is provided a program stored on a computer-readable recording medium, executable by one or more processes on an electronic device, the program may include instructions for performing of: determining, based on prescription information including an exercise plan for a patient being allocated from a doctor terminal, an exercise plan to be provided to the patient; executing the application on a user terminal to which an account of the patient is logged in; providing an exercise list according to the exercise plan to the user terminal on which the application is executed; playing, on the user terminal, an exercise image corresponding to each of a plurality of exercise items according to the plurality of exercise items constituting the exercise list; providing, based on a degree of playback of the exercise image satisfying a preset standard, an evaluation page for performing an evaluation related to the exercise item; and updating the exercise plan based on evaluation information received through the evaluation page.

Meanwhile, there is provided a method of providing exercise therapy according to the present invention to achieve the above-mentioned objects, the method may include: receiving, from a doctor terminal, prescription information related to exercise for a patient; allocating, to an account of the patient, based on the prescription information, an exercise plan including at least one prescribed exercise; receiving, from a patient terminal, an exercise image in which an exercise according to the prescribed exercise is photographed; extracting, from the exercise image, a keypoint corresponding to each of a plurality of preset joint points, using an artificial intelligence posture estimation model; analyzing, using an artificial intelligence motion analysis model, a relative positional relationship between the keypoints, and analyzing, based on the analysis of the positional relationship, an exercise motion of the patient for the prescribed exercise; and transmitting an analysis result of the exercise motion of the patient to the patient terminal, based on the analysis.

Further, the method may further include: outputting the exercise image to the patient terminal in real time, in conjunction with the exercise image being photographed on the patient terminal; and providing a graphic object corresponding to the extracted keypoint that overlaps an area where a subject corresponding to the patient is positioned in the exercise image, so as to allow the patient to recognize a joint point where an analysis is performed on an exercise motion of the patient.

In an embodiment, in the extracting of the keypoint, a visible joint point of the subject that is visible in the exercise image may be specified among the plurality of preset joint points, and in which the specified visible joint point may be extracted as the keypoint.

In an embodiment, the motion analysis model may predict, based on the training data, an invisible joint point of the subject that is not visible in the exercise image among the plurality of preset joint points, and analyze, based on the visible joint point and the invisible joint point, the exercise motion of the patient.

In this case, the keypoints may include keypoints corresponding to the visible joint point and the invisible joint point.

In an embodiment, the training data may include a first data group in which position information for a training target visible joint point of the subject included in a training target image and a training target invisible joint point estimated based on the visible joint point are sequentially listed, and a second data group including data values representing whether each of the training target visible joint point and the training target invisible joint point is visible.

In this case, a sequence of listing of the data values included in the second data group may have the same sequence as a sequence in which the training target visible joint point and the training target invisible joint point are listed.

In an embodiment, in the analyzing of the exercise motion of the patient, a relative positional relationship between the keypoints may be analyzed based on rule information related to the prescribed exercise, and the exercise motion of the patient may be analyzed by judging whether the relative positional relationship between the keypoints satisfies the rule information.

In an embodiment, visual appearances of the graphic objects overlapping the exercise image may be configured to be different depending on whether the relative positional relationship between the extracted keypoints satisfies the rule information.

In an embodiment, the analysis result of the exercise motion of the patient may include: a first analysis result providing the graphic object corresponding to the keypoint that overlaps the exercise image in real time with a different visual appearance based on the rule information, in a state in which the exercise image is being photographed on the patient terminal; and a second analysis result including an evaluation score of the patient for the prescribed exercise based on a keypoint extracted from each of a plurality of frames constituting the exercise image.

In this case, the first analysis result may be generated by a motion analysis model of an application installed on the patient terminal, in which the second analysis result may be generated on a cloud server in conjunction with the application, and in which both the first analysis result and the second analysis result may be transmitted to the doctor terminal.

Further, there is provided a system for providing exercise therapy according to the present invention, the system may include: a communication unit configured to receive, from a doctor terminal, prescription information related to an exercise for a patient; and a control unit configured to allocate, based on the prescription information, an exercise plan including at least one prescribed exercise, to an account of the patient, in which the control unit may be configured to: receive, from the patient terminal, an exercise image in which an exercise according to the prescribed exercise is photographed; extract a keypoint corresponding to each of a plurality of preset joint points from the exercise image; analyze a relative position relationship between the keypoints through an artificial intelligence behavioral analysis model; analyze an exercise motion of the patient for the prescribed exercise, based on the analysis of the positional relationship; and transmit an analysis result of the exercise motion of the patient to the patient terminal.

Further, there is provided a system for providing exercise therapy, the system may include: a communication unit configured to receive, from a doctor terminal, prescription information related to an exercise for a patient; and a control unit configured to allocate, based on the prescription information, an exercise plan including at least one prescribed exercise, to an account of the patient, in which the control unit may be configured to: receive, from the patient terminal, an exercise image in which an exercise according to the prescribed exercise is photographed; analyze, from the exercise image, an exercise motion of the patient for the prescribed exercise, using an artificial intelligence motion analysis model: and transmit an analysis result of the exercise motion of the patient, to the patient terminal.

Further, there is provided a program executable by one or more processes on an electronic device and stored on a computer-readable recording medium, the program may include instructions for performing of: receiving, from a doctor terminal, prescription information related to exercise for a patient; allocating, to an account of the patient, based on the prescription information, an exercise plan including at least one prescribed exercise; receiving, from a patient terminal, an exercise image in which an exercise according to the prescribed exercise is photographed; extracting, from the exercise image, a keypoint corresponding to each of a plurality of preset joint points; analyzing, using an artificial intelligence motion analysis model, a relative positional relationship between the keypoints, and analyzing, based on the analysis of the positional relationship, an exercise motion of the patient for the prescribed exercise; and transmitting an analysis result of the exercise motion of the patient to the patient terminal, based on the analysis.

Meanwhile, there is provided a method of estimating an exercise posture according to the present invention, the method may include: receiving, from a user terminal, an exercise image; analyzing, based on posture estimation information extracted from a posture estimation model trained using a training data set 400 including position information for a joint point, an exercise motion related to a specific exercise motion of a user included in the exercise image; and providing, based on a completion of the analysis, an exercise motion analysis result of the user related to the specific exercise motion of the user to the user terminal, in which the position information for the joint point included in the training data set may be position information for each of a plurality of predesignated training target joint points among joint points of a subject included in a training target exercise image, and in which the training data set may be configured with data extracted from the training target exercise image.

In an embodiment, the training data set may be configured with a plurality of data groups, corresponding respectively to different information attributes, in which a first data group of the plurality of data groups may include position information on each of the plurality of training target joint points, and in which, in the position information included in the first data group, coordinate information of each of the plurality of predesignated training target joint points in the training target exercise image may be included in a paired form.

In an embodiment, the position information included in the first data group may be defined as different types of information based on whether the plurality of training target joint points are visible in the training target image, and in which the definition of a type for the position information may be determined by data values of data included in a second data group different from the first data group.

In an embodiment, the posture estimation model may, based on the data value included in the second data group, set a training weight for the position information of each of the plurality of training target joint points included in the first data group differently.

In an embodiment, the position information of each of the plurality of training target joint points included in the first data group may be arranged sequentially within the first data group, based on a predefined sequence among the plurality of training target joint points, and in which the data values included in the second data group may be arranged within the second data group in the same sequence as the predefined sequence in which the position information of each of the plurality of training target joint points is arranged, so as to represent whether each of the plurality of training target joint points is visible.

In an embodiment, the training data set may further include a third data group including data values related to a photographing direction for the subject, in which the data values included in the third data group may be configured to have different data values depending on the photographing direction of the subject with respect to a camera that photographs the subject.

In this case, the posture estimation model may be configured to be trained in consideration of the photographing direction of the subject through the training data set having different data values according to the photographing direction of the subject, and in which an exercise motion analysis result of the user may be a result of analyzing a specific exercise motion of the user based on posture estimation information extracted in consideration of the photographing direction of the user included in the exercise image in the posture estimation model.

In an embodiment, the exercise image received from the user terminal and the training target exercise image may correspond to the specific exercise motion having an identical exercise code, and in which the training data set may include a fourth data group including an exercise code matched to the specific exercise motion performed by the subject in the training target exercise image.

Further, the training target exercise image may be a motion image, and in which the training data set may be configured with training data extracted centered on the subject included in the training target exercise image, from each of a plurality of standard frames selected based on a preset standard among a plurality of frames constituting the training target exercise image.

There is provided a system for estimating an exercise posture according to the present invention, the system may include: a posture estimation model configured to extract posture estimation information using a training data set including position information for a joint point; a motion analysis model configured to analyze, using the posture estimation information, an exercise motion related to a specific exercise motion of a user included in an exercise image targeted for analysis; and a service server configured to provide, based on a completion of the analysis, an exercise motion analysis result of the user related to the specific exercise motion of the user to the user terminal, in which the position information for the joint point included in the training data set may be position information of each of a plurality of predesignated training target joint points among joint points of a subject included in the training target exercise image, and in which the training data set may be configured with data extracted from the training target exercise image.

Further, there is provided a program executed by one or more processes on an electronic device and stored on a computer-readable recording medium, the program may include instructions to perform of: receiving, from a user terminal, an exercise image; analyzing, based on posture estimation information extracted from a posture estimation model trained using a training data set including position information for a joint point, an exercise motion related to a specific exercise motion of a user included in the exercise image; and providing, based on a completion of the analysis, an exercise motion analysis result of the user related to the specific exercise motion of the user to the user terminal, in which the position information for the joint point included in the training data set may be position information for each of a plurality of predesignated training target joint points among joint points of a subject included in a training target exercise image, and in which the training data set may be configured with data extracted from the training target exercise image.

Meanwhile, there is provided a method for providing cognitive behavioral therapy according to the present invention, the method may include: providing, on a user terminal, a plurality of survey data for diagnosing a state of a pain patient; receiving, through the user terminal, survey response data for the plurality of survey data; detecting, based on the survey response data, state information of the pain patient related to pain duration and a degree of cognitive distortion of the pain patient; specifying a user group corresponding to the state information of the pain patient among a plurality of user groups categorized according to the pain duration and degree of cognitive distortion; determining an initial therapy protocol corresponding to the user group among a plurality of therapy protocols; and providing sequentially a plurality of specific therapy programs included in the initial therapy protocol to the user terminal, according to a therapy week set for each of the plurality of specific therapy programs.

The therapy protocol according to the present invention may be understood as a therapy process configured with a plurality of therapy programs, each matched to a different topic. Further, the specific therapy program may be configured to include at least one therapy module related to a specific topic matched to the specific therapy program. Here, the therapy module may be understood as a detailed category for cognitive behavioral therapy of the pain patient for a specific topic. According to an embodiment, a server may have a plurality of therapy programs stored that are each matched to a different topic, in which the plurality of therapy programs may be configured to include at least one therapy module related to the topic matched to each of the plurality of therapy programs, and in which each of the plurality of therapy protocols may be configured with at least one different therapy program or therapy module depending on characteristics of pain duration and degree of cognitive distortion of a user group matched to each of the plurality of therapy protocols.

In this case, the plurality of user groups may include: a first user group having acute pain and high cognitive distortion, in relation to the characteristics of pain duration and degree of cognitive distortion; a second user group having acute pain and low cognitive distortion, in relation to the characteristics of pain duration and degree of cognitive distortion; a third user group having chronic pain and high cognitive distortion, in relation to the characteristics of pain duration and degree of cognitive distortion; and a fourth user group having chronic pain and low cognitive distortion, in relation to the characteristics of pain duration and degree of cognitive distortion, in which a reference for distinguishing between acute pain and chronic pain may be determined by whether the pain duration exceeds a reference period, and in which a reference for distinguishing between high cognitive distortion and low cognitive distortion may be determined by whether a score collected based on a plurality of questions related to cognitive distortion exceeds a reference score.

According to an embodiment, the providing sequentially to the user terminal may include: providing sequentially, to the user terminal, specific therapy modules constituting the plurality of specific therapy programs, according to a therapy week set for each of the plurality of specific therapy programs; and collecting, from the user terminal, therapy response data from the specific therapy modules, and may further include: updating, using the therapy response data, the initial therapy protocol.

According to an embodiment, the updating of the initial therapy protocol may be configured to update the initial therapy protocol using initial therapy response data required by the therapy modules provided for each therapy week of a preset initial therapy period among a preset overall therapy period during which the plurality of specific therapy programs are scheduled to be provided to the user terminal, and in which the updating of the initial therapy protocol may be accomplished through changing at least one of remaining therapy programs allocated for a remaining therapy period of the overall therapy period excluding the initial therapy period and therapy modules constituting the remaining therapy programs.

According to an embodiment, the updating of the initial therapy protocol may include: analyzing, using the initial therapy response data, the state of the pain patient for each of a plurality of different analysis categories related to at least one of emotion, pain, insomnia, cognitive distortion, or stress; specifying, using an analysis result, a category of the plurality of analysis categories for which a problematic symptom of the pain patient satisfies a preset reference; and changing at least one of the remaining therapy programs allocated for the remaining therapy period or the therapy modules constituting the remaining therapy programs, to be related to the specified category.

According to an embodiment, as a result of updating the initial therapy protocol, at least some of example sentences provided from the therapy modules constituting the remaining therapy programs may be changed to be related to the specified category.

According to an embodiment, the updating of the initial therapy protocol may include: performing to judge, based on the therapy response data, whether to maintain the preset overall therapy period for which the plurality of specific therapy programs are scheduled to be provided to the user terminal; and performing, based on a judgment result, to update the preset overall therapy period, and in which the update of the preset overall therapy period may include shortening the overall therapy period to a period shorter than the overall therapy period or lengthening the overall therapy period to a period longer than the overall therapy period. According to an embodiment, each of the plurality of specific therapy programs may include a worksheet module configured to review at least one of a degree of pain, pain duration, a mental health state, or a physical health state of the pain patient, and in which in the providing sequentially to the user terminal, user response information for a worksheet module provided in a therapy week prior to a current therapy week of the plurality of specific therapy programs may be provided in priority before a therapy program corresponding to the current therapy week of the plurality of specific therapy programs is provided, in order to make the pain patient aware of a past state of the pain patient.

Further, there may be provided a system for providing cognitive behavioral therapy according to the present invention, the system may include: a communication unit configured to perform communication with a user terminal; a storage unit configured to store a plurality of therapy programs each matched to a different topic; and a control unit configured to provide a plurality of survey data for diagnosing a state of a pain patient on the user terminal, in which the control unit may be configured to: receive, through the communication unit, response data to the plurality of survey data from the user terminal; detect, based on the response data, state information of the pain patient related to the pain duration and degree of cognitive distortion of the pain patient; specify a user group corresponding to the state information of the pain patient among a plurality of user groups categorized according to the pain duration and degree of cognitive distortion; determine an initial therapy protocol corresponding to the user group among a plurality of therapy protocols; and provide a plurality of specific therapy programs included in the initial therapy protocol, sequentially to the user terminal, according to a therapy week set for each of the plurality of specific therapy programs.

Further, there may be provided a program executed by one or more processes on an electronic device and stored on a computer-readable recording medium, in which the program may include instructions for performing of: providing, on a user terminal, a plurality of survey data for diagnosing a state of a pain patient; receiving, through the user terminal, response data to the plurality of survey data; detecting, based on the response data, state information of the pain patient related to the pain duration and degree of cognitive distortion of the pain patient; specifying a user group corresponding to the state information of the pain patient among a plurality of user groups categorized according to the pain duration and degree of cognitive distortion; determining an initial therapy protocol corresponding to the user group among a plurality of therapy protocols; and providing a plurality of specific therapy programs included in the initial therapy protocol to the user terminal sequentially, according to a therapy week set for each of the plurality of specific therapy programs.

### Advantageous Effects

As described above, the method and system for providing digitally-based musculoskeletal rehabilitation therapy according to the present invention can provide an exercise plan for musculoskeletal rehabilitation therapy to a patient through an application based prescription information including an exercise plan for the patient being allocated from the doctor terminal.

In particular, the method and system for providing digitally-based musculoskeletal rehabilitation therapy according to the present invention can play an exercise image corresponding to each of the plurality of exercise items, according to the plurality of exercise items constituting an exercise list, on the user terminal on which the application is executed. This allows a doctor to prescribe to a patient, and a patient to proceed rehabilitation through an exercise plan based on the doctor's prescription, even if the doctor and patient do not meet in person for rehabilitation therapy for a musculoskeletal disease, thereby resolving spatial, temporal, and economic constraints on the musculoskeletal rehabilitation therapy and increasing accessibility to the exercise therapy.

Further, the method and system for providing digitally-based musculoskeletal rehabilitation therapy according to the present invention can provide an evaluation page for performing an evaluation related to an exercise item based on a degree of playback of an exercise image satisfying a preset standard, and update an exercise plan based on evaluation information received through the evaluation page. This allows the patient to perform the exercise plan, provide appropriate feedback, and receive personalized rehabilitation therapy in which feedback is applied. In particular, the patient can be provided with individualized rehabilitation exercise therapy, such as adjusting the difficulty of exercise items according to the patient's state, and excluding exercise items that are difficult for the patient.

Further, the method and system for providing digitally-based musculoskeletal rehabilitation therapy according to the present invention can provide a cognitive behavioral therapy plan in conjunction with a rehabilitation exercise plan, thereby providing the patient with therapy for mental health as well as the rehabilitation site.

In particular, the method and system for providing digitally-based musculoskeletal rehabilitation therapy according to the present invention can receive, through the user terminal, survey response data for a plurality of survey data, and detect, based on the survey response data, state information on the patient related to pain duration and a degree of cognitive distortion of the patient. Further, based on the state information of the patient, a therapy protocol for personalized cognitive behavioral therapy can be provided to the patient. As a result, the method and system for providing rehabilitation therapy according to the present invention can provide the patient with a personalized cognitive behavioral therapy program for the patient in consideration of the pain duration and degree of cognitive distortion of the patient even if the patient has the same musculoskeletal disease, rather than providing the patient with a uniform cognitive behavioral therapy based on the musculoskeletal disease of the patient. Further, the patient can be provided with the personalized cognitive behavioral therapy that fits his/her state.

Further, the method and system for providing digitally-based musculoskeletal rehabilitation therapy according to the present invention can provide a plurality of therapy programs sequentially in conjunction with an exercise plan during a rehabilitation period. This allows the patient to complete rehabilitation therapy without dropping out, by systematically providing cognitive behavioral therapy along with the rehabilitation exercise.

Further, the method and system for providing digitally-based musculoskeletal rehabilitation therapy according to the present invention can update the therapy program to help the cognitive behavioral therapy of the patient based on therapy response data collected in the process of performing the cognitive behavioral therapy, so that the updated cognitive behavioral therapy can be provided in consideration of the patient's remission, rather than the initially determined method of cognitive behavioral therapy being provided continuously.

### BRIEF DESCRIPTION OF THE DRAWINGS

FIG. 1 is a conceptual view for describing a system for providing digitally-based musculoskeletal rehabilitation therapy according to the present invention.

FIGS. 2A, 2B, and 2C are conceptual views for describing a method of digitally-based musculoskeletal rehabilitation therapy according to the present invention.

FIG. 3 is a flowchart for describing a method of providing digitally-based musculoskeletal rehabilitation therapy according to the present invention.

FIG. 4 is a conceptual view for describing a method of providing musculoskeletal rehabilitation therapy and cognitive behavioral therapy in conjunction with each other in the present invention.

FIGS. 5A, 5B, 5C, 6, and 7 are concepts for describing a method of providing an exercise plan for personalized musculoskeletal rehabilitation therapy for a patient in the present invention.

FIGS. 8A, 8B, 8C, 8D, 8E, 8F, 8G, 8H, 8I, 8J, 8K, 8L, and 8M are concepts for describing a method of providing cognitive behavioral therapy in the present invention.

FIGS. 9A and 9B are concepts for describing a method of providing an AI functional evaluation service in the present invention.

FIG. 10 is a concept for describing a rehabilitation therapy report provided in the present invention.

FIG. 11 is a conceptual view for describing a system for providing exercise therapy according to the present invention.

FIGS. 12 and 13 are flowcharts for describing a method of providing exercise therapy according to the present disclosure.

FIGS. 14A and 14B are conceptual views for describing a doctor's prescription.

FIGS. 15 and 16 are conceptual views for describing a method of analyzing an exercise motion of a patient from an exercise image.

FIGS. 17, 18A, 18B, 18C, 18D, 18E, and 18F are conceptual views for describing an artificial intelligence posture estimation model.

FIGS. 19 and 20 are conceptual views for describing an application example that provides a motion analysis result of a user.

FIGS. 21A, 21B, and 21C are conceptual views for describing a user environment in which an exercise motion analysis result of a patient is provided.

### DETAILED DESCRIPTION OF THE INVENTION

Hereinafter, exemplary embodiments disclosed in the present specification will be described in detail with reference to the accompanying drawings. The same or similar constituent elements are assigned with the same reference numerals regardless of reference numerals, and the repetitive description thereof will be omitted. The suffixes “module”, “unit”, “part”, and “portion” used to describe constituent elements in the following description are used together or interchangeably in order to facilitate the description, but the suffixes themselves do not have distinguishable meanings or functions. In addition, in the description of the exemplary embodiment disclosed in the present specification, the specific descriptions of publicly known related technologies will be omitted when it is determined that the specific descriptions may obscure the subject matter of the exemplary embodiment disclosed in the present specification. In addition, it should be interpreted that the accompanying drawings are provided only to allow those skilled in the art to easily understand the embodiments disclosed in the present specification, and the technical spirit disclosed in the present specification is not limited by the accompanying drawings, and includes all alterations, equivalents, and alternatives that are included in the spirit and the technical scope of the present disclosure.

The terms including ordinal numbers such as “first,” “second,” and the like may be used to describe various constituent elements, but the constituent elements are not limited by the terms. These terms are used only to distinguish one constituent element from another constituent element.

When one constituent element is described as being “coupled” or “connected” to another constituent element, it should be understood that one constituent element can be coupled or connected directly to another constituent element, and an intervening constituent element can also be present between the constituent elements. When one constituent element is described as being “coupled directly to” or “connected directly to” another constituent element, it should be understood that no intervening constituent element is present between the constituent elements.

Singular expressions include plural expressions unless clearly described as different meanings in the context.

In the present application, it should be understood that terms “including” and “having” are intended to designate the existence of characteristics, numbers, steps, operations, constituent elements, and components described in the specification or a combination thereof, and do not exclude a possibility of the existence or addition of one or more other characteristics, numbers, steps, operations, constituent elements, and components, or a combination thereof in advance.

The present invention is directed to providing digitally-based rehabilitation therapy and cognitive behavioral therapy associated with the rehabilitation therapy to a patient with a muscle disease, and more particularly to a method and system for providing personalized rehabilitation therapy based on an indication of a patient. According to the present invention, a doctor (or healthcare provider) may provide an exercise plan corresponding to an indication that a patient has, and a user may perform the exercise plan and provide feedback to the system according to the present invention. The system according to the present invention may update the exercise plan appropriately to reflect the patient's feedback, thereby ensuring that the effect of the therapy according to the indication of the patient is maximized.

Here, the term, indication, may be understood as a symptom or clinical situation that requires specific therapy or examination, such as a disease or symptom of a patient. Further, the term, indication, may be understood as a disease or symptom of a patient in need of therapy or rehabilitation therapy.

In the present invention, for rehabilitation therapy of a musculoskeletal patient, at least one of rehabilitation therapy or cognitive behavioral therapy may be provided.

For convenience of description, the present invention is described with a focus on a “musculoskeletal disease,” but is not necessarily limited thereto. That is, the rehabilitation therapy described in the present invention may include therapy for a patient in need of rehabilitation therapy due to a musculoskeletal disease, as well as therapy for a patient due to a variety of diseases (e.g., cancer, diabetes, hypertension, etc.).

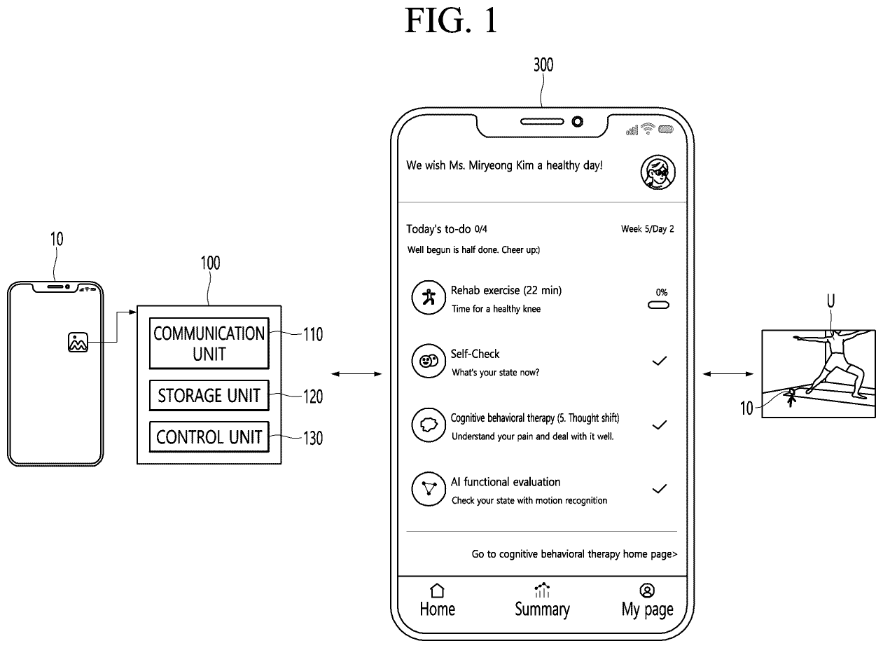

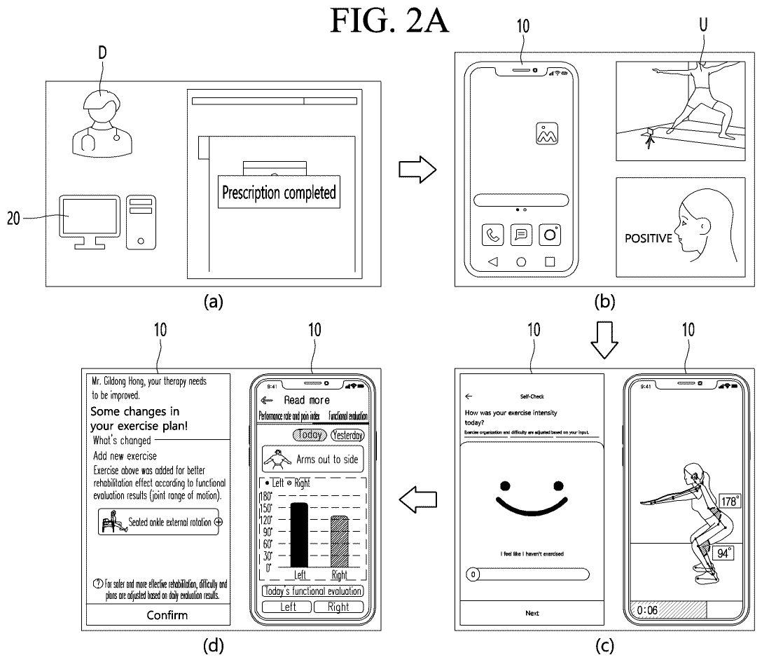

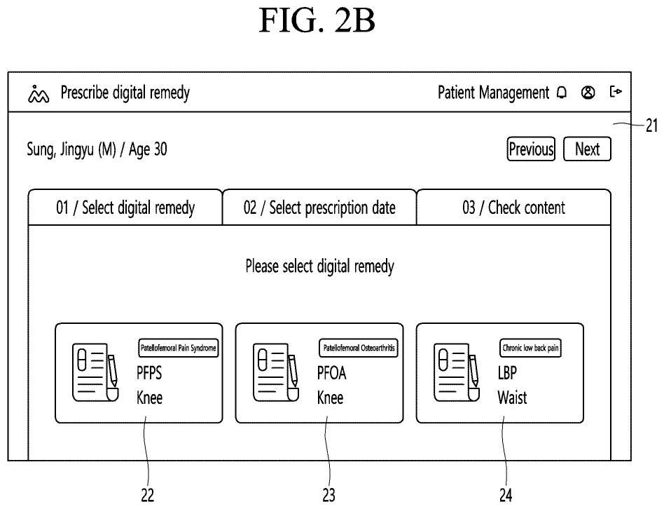

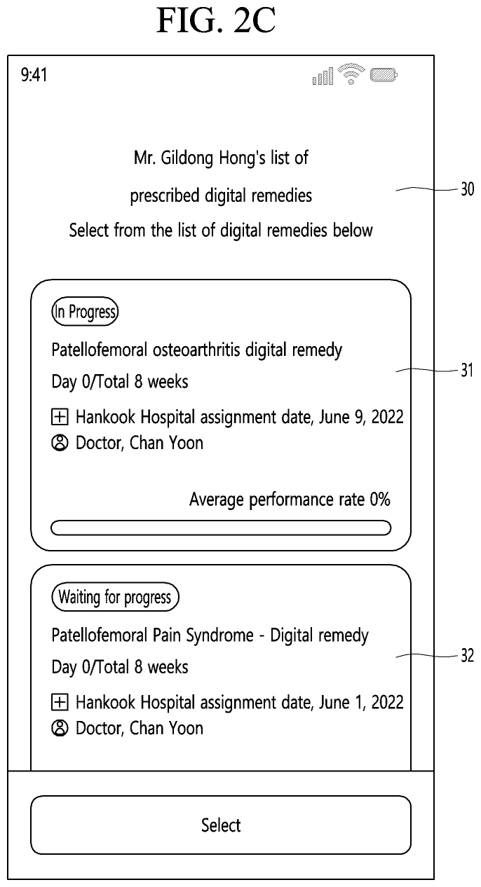

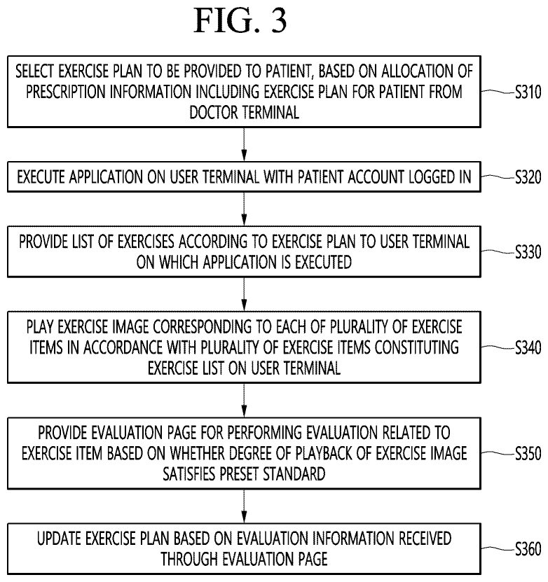

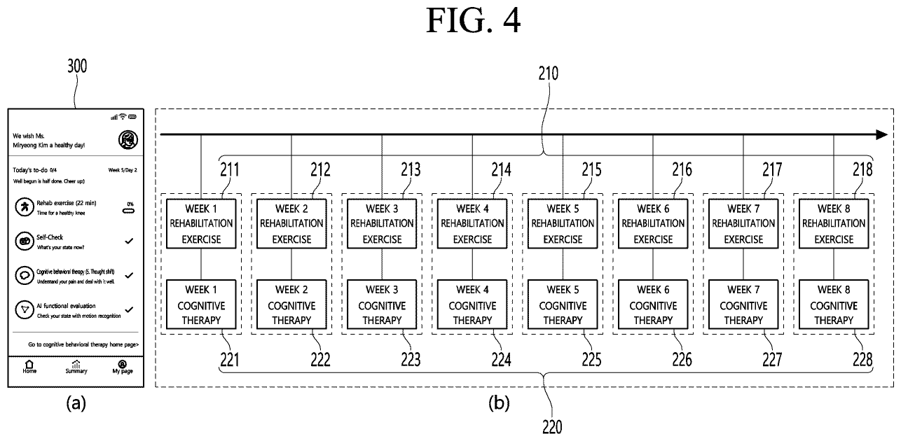

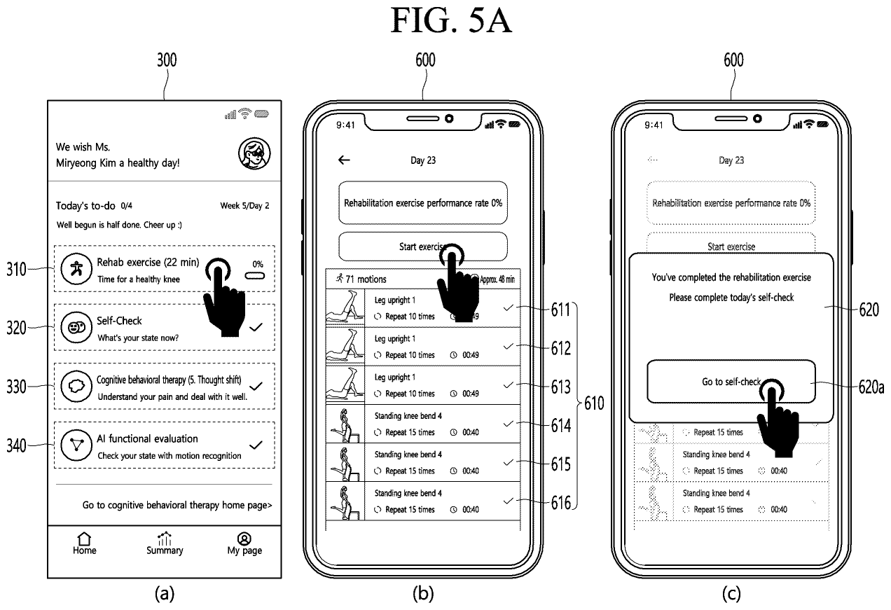

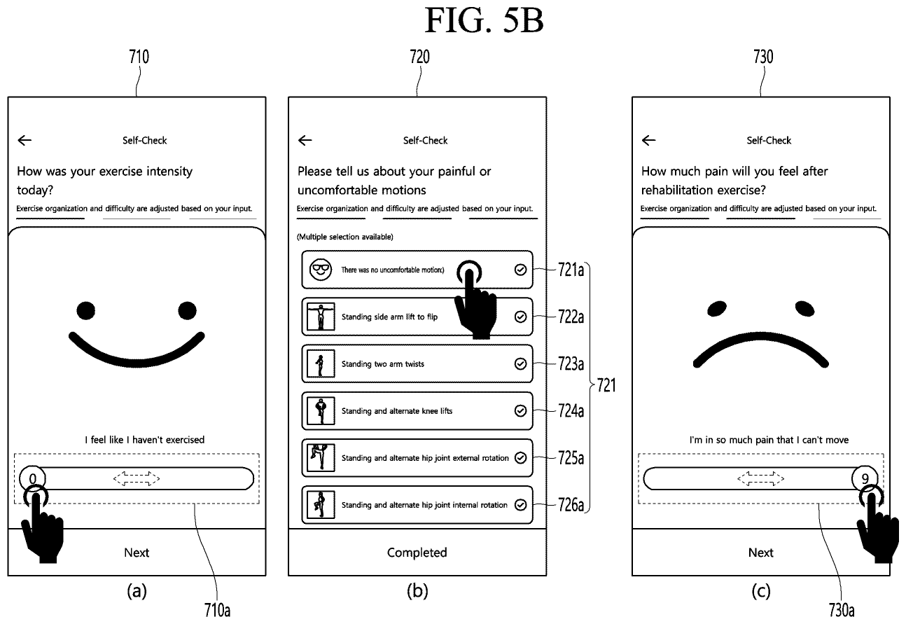

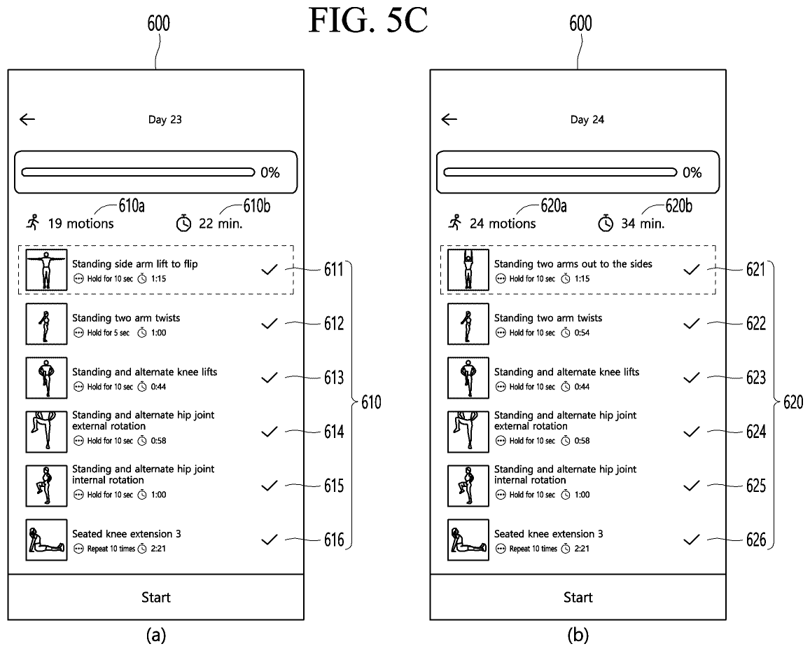

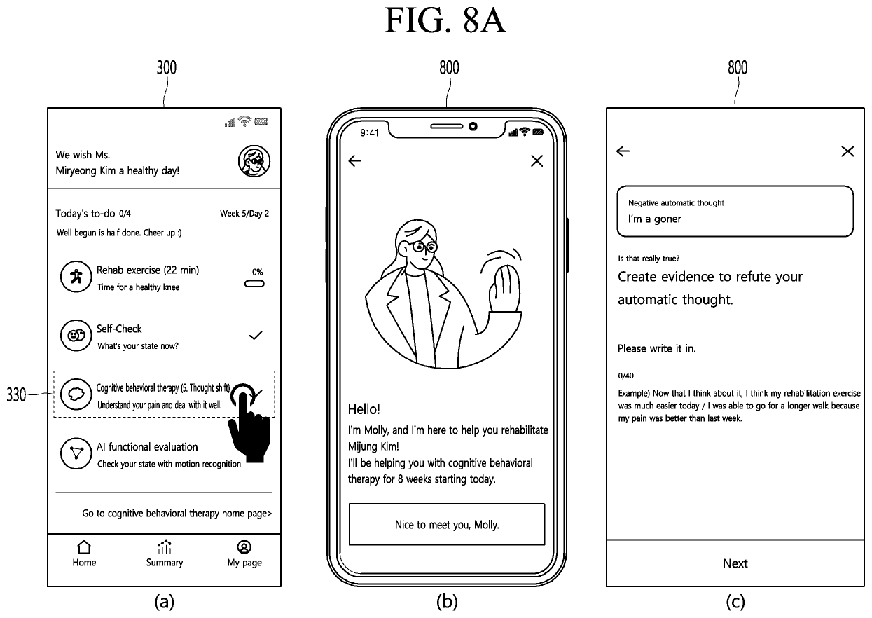

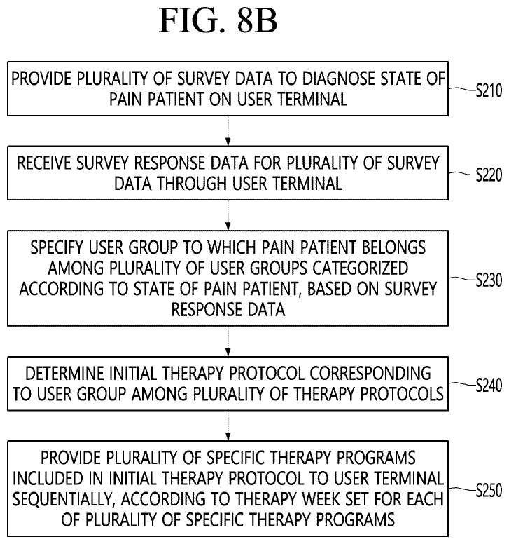

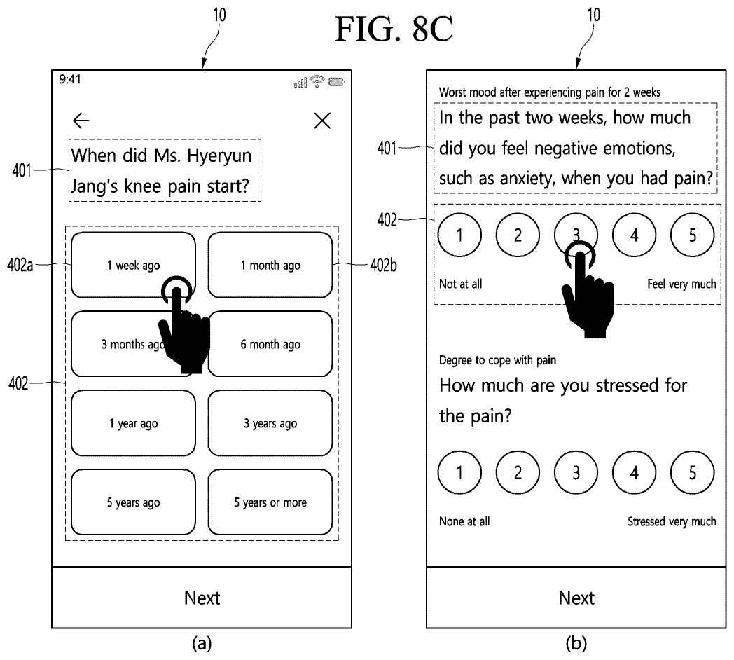

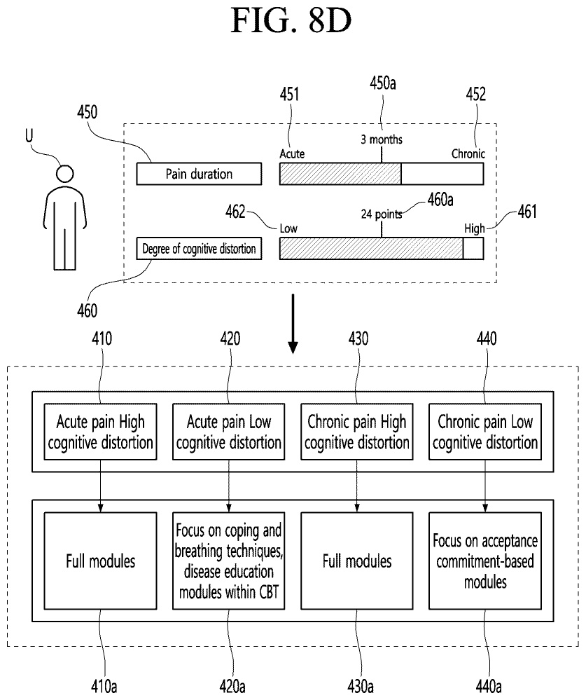

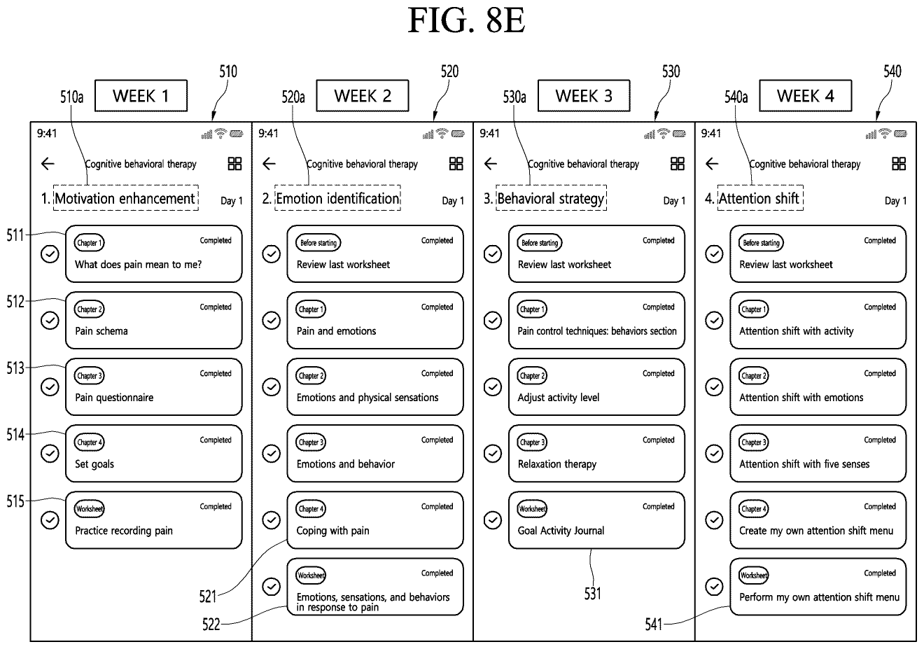

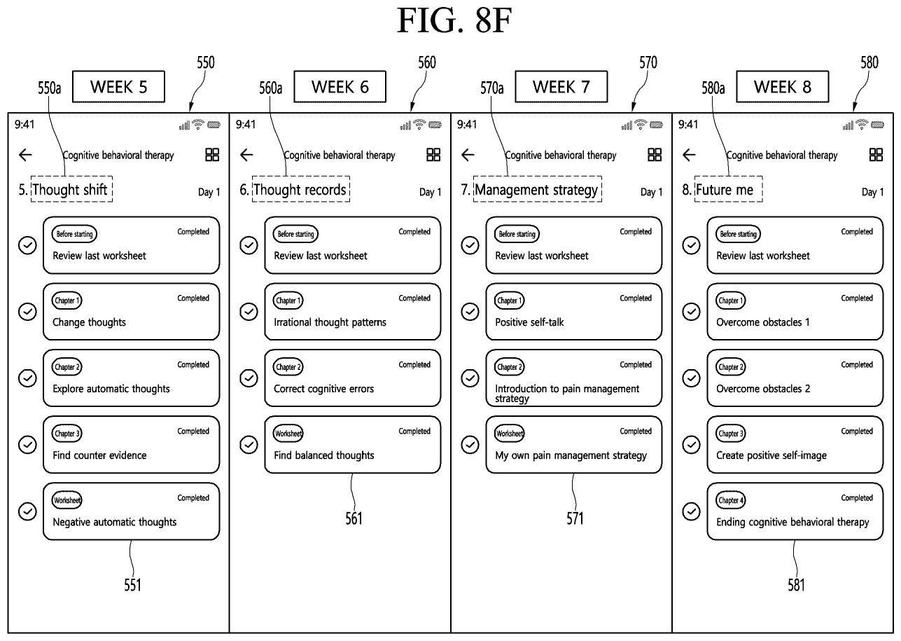

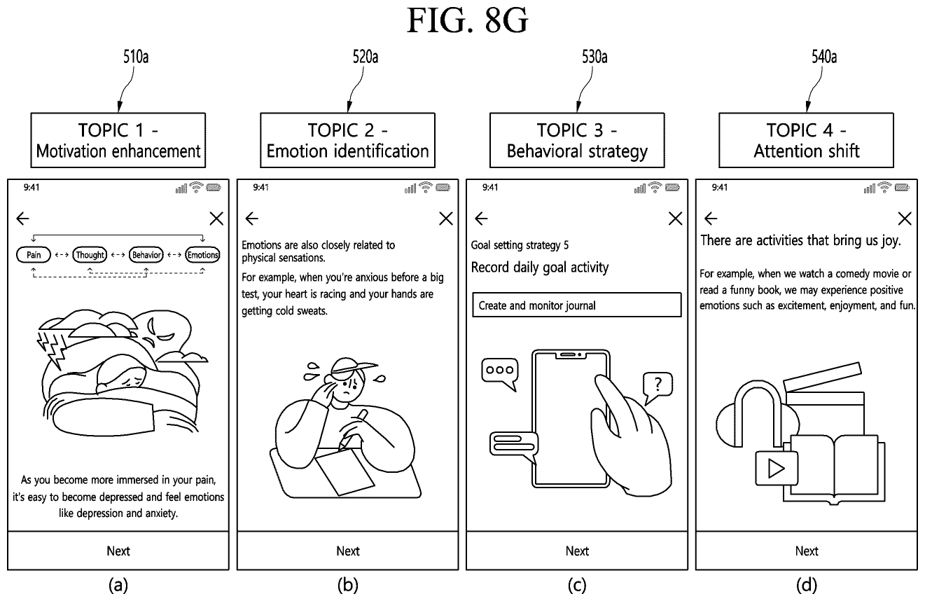

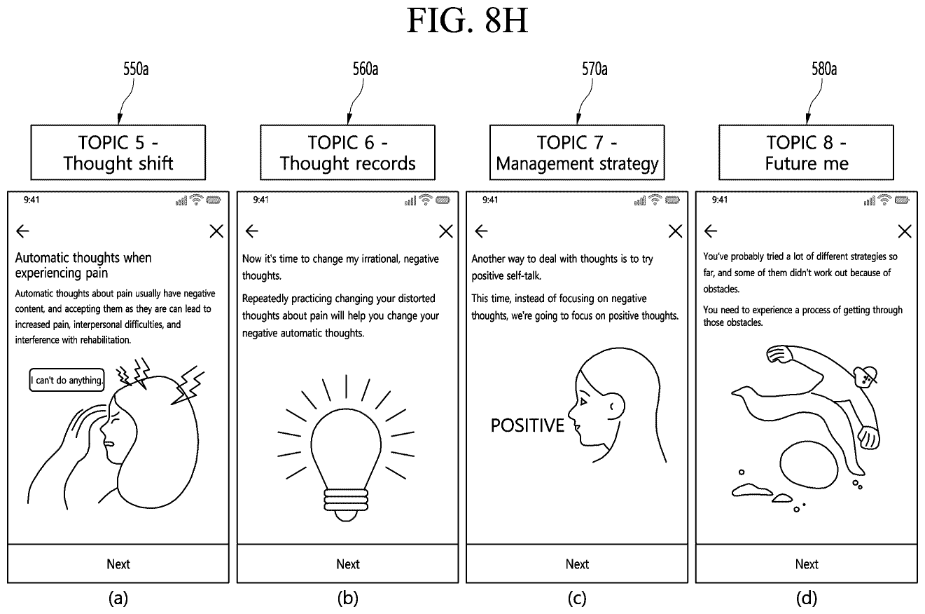

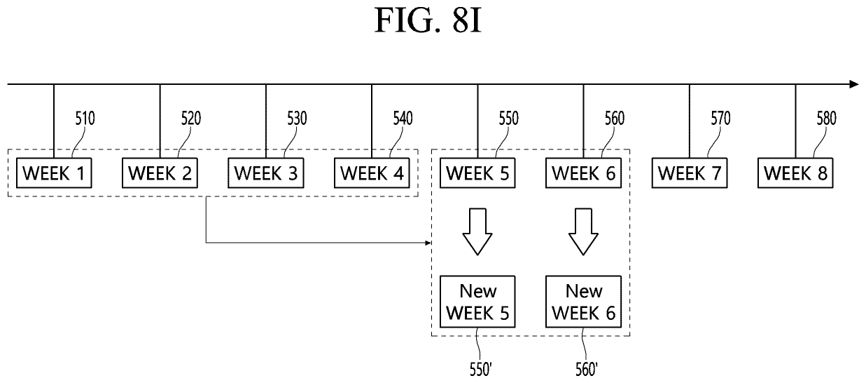

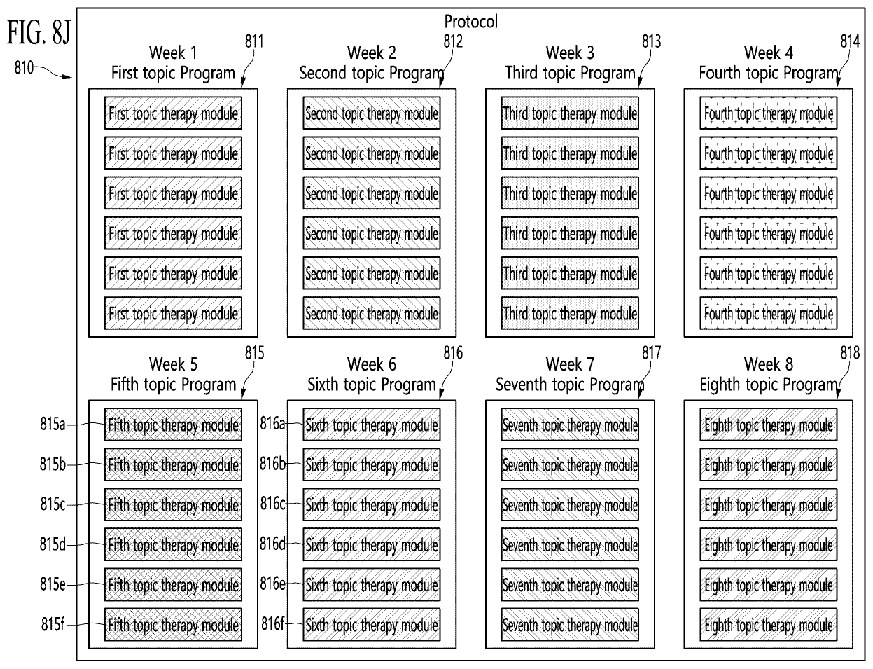

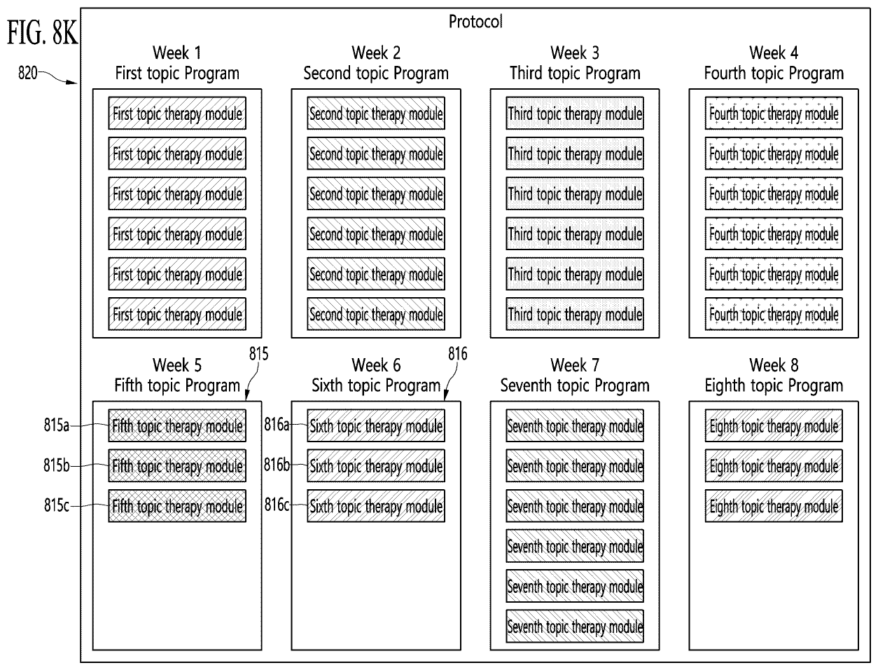

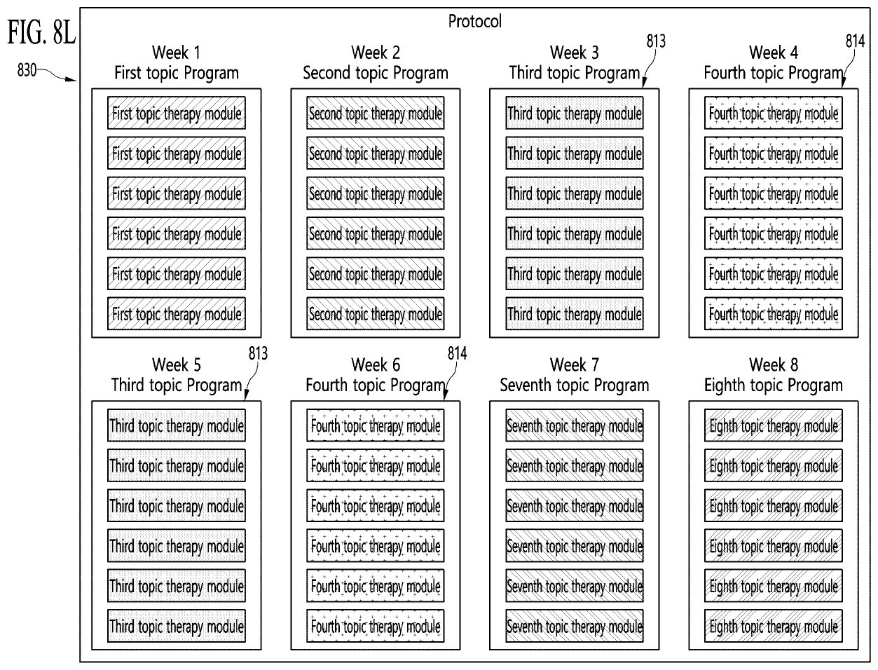

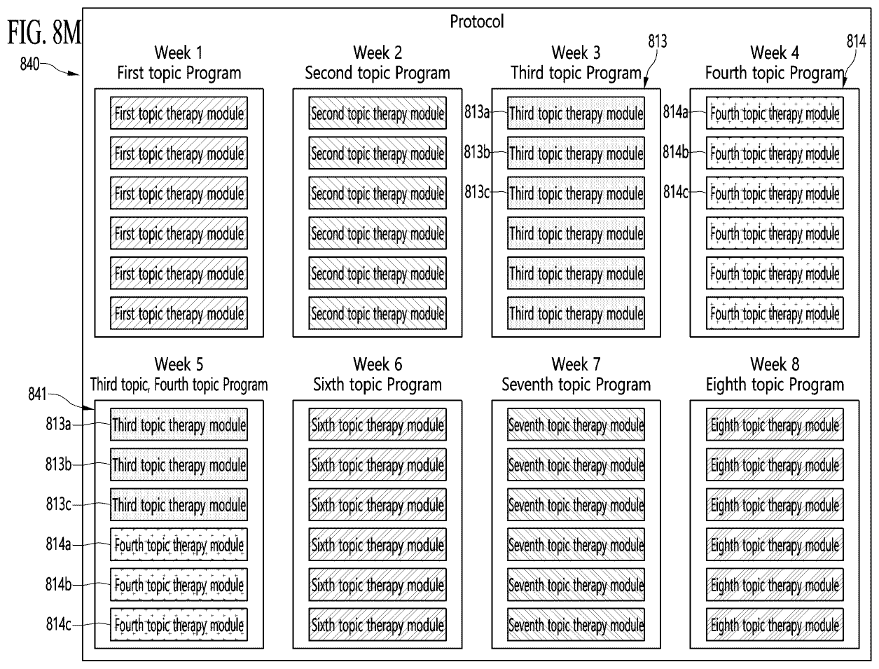

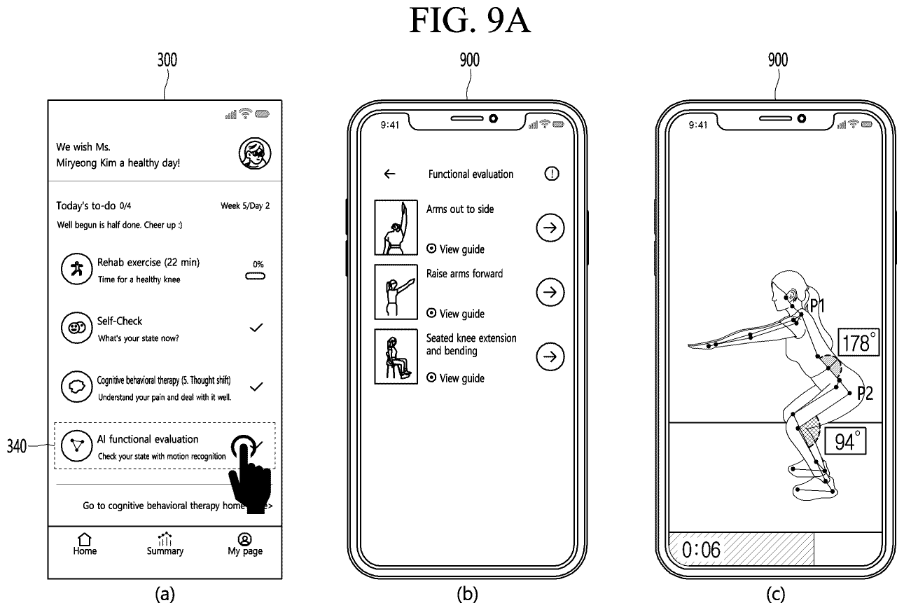

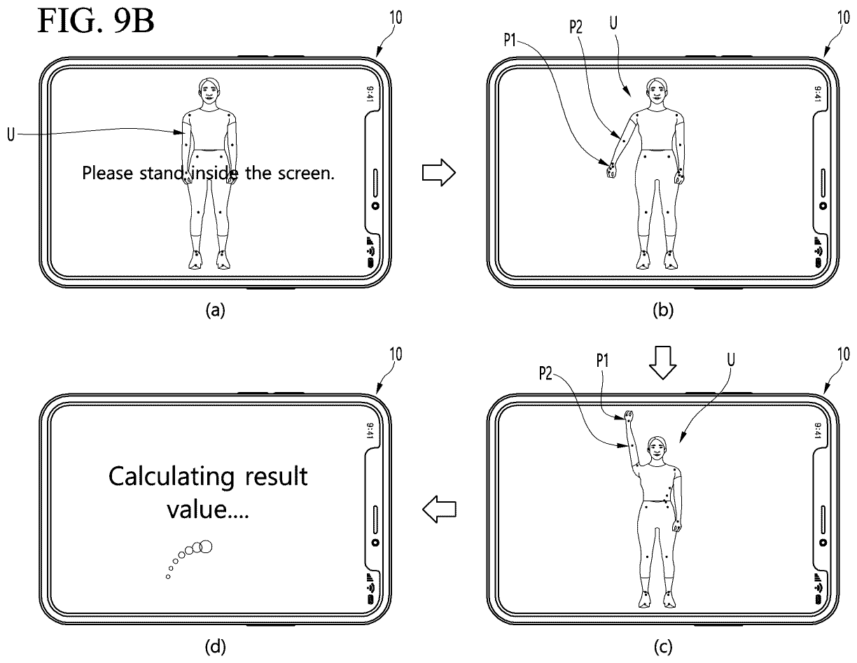

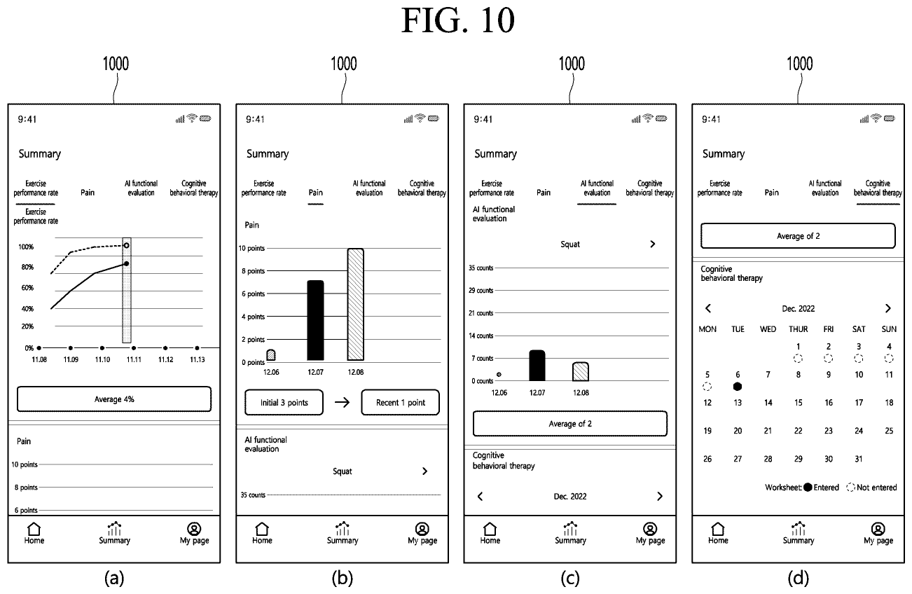

Hereinafter, with reference to the accompanying drawings, a method and system for providing personalized rehabilitation therapy for a musculoskeletal patient on a digital basis will be described in detail. FIG. 1 is a conceptual view for describing a system for providing digitally-based musculoskeletal rehabilitation therapy according to the present invention. FIGS. 2A, 2B, and 2C are conceptual views for describing a method of digitally-based musculoskeletal rehabilitation therapy according to the present invention, FIG. 3 is a flowchart for describing a method of providing digitally-based musculoskeletal rehabilitation therapy according to the present invention, FIG. 4 is a conceptual view for describing a method of providing musculoskeletal rehabilitation therapy and cognitive behavioral therapy in conjunction with each other in the present invention, FIGS. 5A, 5B, 5C, 5D, 6, and 7 are concepts for describing a method of providing an exercise plan for personalized musculoskeletal rehabilitation therapy for a patient in the present invention, FIGS. 8A, 8B, 8C, 8D, 8E, 8F, 8G, 8H, 8I, 8J, 8K, 8L, and 8M are concepts for describing a method of providing cognitive behavioral therapy in the present invention, FIGS. 9A and 9B are concepts for describing a method of providing an AI functional evaluation service in the present invention, and FIG. 10 is a concept for describing a rehabilitation therapy report provided in the present invention.

As illustrated in FIG. 1, a system for providing digitally-based musculoskeletal rehabilitation therapy according to the present invention (hereinafter referred to as “rehabilitation therapy provision system”, 100) may be implemented as an application or software.

According to a software implementation of a rehabilitation therapy provision system 100 according to the present invention, embodiments such as the procedures and functions described herein may be implemented as separate software therapy modules. Each of the software therapy modules may perform one or more of the functions and operations described herein.

As such, the software-implemented rehabilitation therapy provision system 100 may be downloaded to a user terminal 10 through a program that allows applications to be downloaded to the user terminal 10 (e.g., a play store), or may be implemented through an initial installation program on the user terminal 10. In this case, a communication unit 110, a storage unit 120, and a control unit 130 according to the present invention may be used as constituent elements of the user terminal 10.

In the present invention, the terminal 10 may also be referred to as a “mobile terminal” or an “electronic device”, and the terminal described herein may include a cell phone, a smart phone, a laptop computer, a digital broadcasting terminal, a personal digital assistant (PDA), a portable multimedia player (PMP), a navigation device, a slate PC, a tablet PC, an Ultrabook, a wearable device (e.g., a smartwatch, a smart glass, a head mounted display (HMD)), and the like.

Meanwhile, the rehabilitation therapy provision system 100 may exist inside a server built to perform a specific purpose (e.g., a function related to providing rehabilitation therapy) (hereinafter referred to as a server) separate from the user terminal 10, or may exist as a system separate from the server. When the rehabilitation therapy provision system 100 exists inside the server, a musculoskeletal rehabilitation therapy service may be provided by at least one constituent element of the communication unit 110, storage unit 120, and control unit 130 located inside the server, or by a constituent module that performs a function similar to each of the constituent elements. In this case, the application may communicate with the server to provide services related to the musculoskeletal rehabilitation therapy to the user terminal 10 on which the application is installed. Further, the rehabilitation therapy provision system 100 according to the present invention may provide a method of providing rehabilitation therapy according to the present invention to the user terminal 10 in conjunction with an external server.

Meanwhile, the rehabilitation therapy provision system 100 according to the present invention may provide rehabilitation therapy and cognitive behavioral therapy services to a patient based on a doctor's prescription for the patient, and may provide personalized rehabilitation therapy to the patient based on the patient's feedback on the rehabilitation therapy.

As illustrated in (a) of FIG. 2A, in the present invention, a doctor terminal 20 may receive prescription information prescribed by a doctor D for rehabilitation therapy of a musculoskeletal patient. In the present invention, based on the prescription information received from the doctor terminal 20, the rehabilitation therapy and cognitive behavioral therapy services may be provided through the user terminal 10 on which the application is installed. A patient U may execute the application on the user terminal 10, perform a rehabilitation exercise while viewing a provided exercise image, and proceed with cognitive behavioral therapy (see (b) of FIG. 2A). Further, in the present invention, an evaluation service that allows the patient U who has completed the rehabilitation therapy to proceed with an evaluation of the rehabilitation therapy and an analysis service for an exercise motion of the patient may be provided (see (c) of FIG. 2A). Further, in the present invention, personalized rehabilitation therapy may be provided to the patient by updating the rehabilitation therapy provided to the patient based on the patient's feedback on the rehabilitation therapy (see (d) of FIG. 2A).

As described above, the rehabilitation therapy provision system 100 according to the present invention can provide a personalized musculoskeletal rehabilitation therapy service for a patient by continuously updating the rehabilitation therapy provided to the musculoskeletal patient based on the patient's feedback.

Accordingly, the rehabilitation therapy provision system 100 according to the present invention may be referred to as a “digital rehabilitation therapy solution”, a “digital rehabilitation exercise solution”, a “contactless rehabilitation therapy solution”, a “contactless rehabilitation exercise solution”, a “mobile rehabilitation therapy program”, a “mobile rehabilitation exercise program”, and an “application for digital therapy of a musculoskeletal patient”.

Meanwhile, the patient U may perform, through an application or a webpage provided by the rehabilitation therapy provision system 100 according to the present invention, a rehabilitation exercise according to an exercise plan prescribed by a doctor, and may perform an evaluation on the exercise plan after the rehabilitation exercise.

In this case, the patient U may have a registered user account in the rehabilitation therapy provision system 100 according to the present invention. For convenience of description, an account of a user who is a patient is referred to herein as a “patient account” (or user account).

The “account” described above may be created through a page associated with the rehabilitation therapy provision system 100. In contrast, it is also possible that the “account” is created in at least one different system associated with the rehabilitation therapy provision system 100 according to the present invention.

Accordingly, in the present specification, all accounts based on the rehabilitation therapy provision system 100 according to the present invention will be referred to as an “account registered in the rehabilitation therapy provision system 100 according to the present invention” without distinguishing the system in which the account is issued.

Meanwhile, a doctor may provide a prescription related to rehabilitation therapy to the musculoskeletal patient U through the doctor terminal 20. In this case, the doctor D may have a registered user account in the rehabilitation therapy provision system 100 according to the present invention. For convenience of description, an account of a user who is a doctor is referred to herein as a “doctor account”. Further, the user terminal logged in with the doctor account may be described to be referred to as the doctor terminal 20.

Hereinafter, the rehabilitation therapy provision system 100 according to the present invention will be described according to an embodiment implemented as an application on the user terminal 10.

As illustrated in FIG. 1, the digitally-based musculoskeletal rehabilitation therapy provision system 100 according to the present invention may be configured to include at least one of the communication unit 110, the storage unit 120, or the control unit 130. In this case, the rehabilitation therapy provision system 100 according to the present invention is not limited to the constituent elements described above, and may further include constituent elements that serve the same function as the devices described herein.

The communication unit 110 may include one or more therapy modules to enable wireless or wired communication between the rehabilitation therapy provision system 100 and the user terminal 10, or between the rehabilitation therapy provision system 100 and an external server. In addition, the communication unit 110 may include one or more communication modules that connect the rehabilitation therapy provision system 100 to one or more networks.

Specifically, the communication unit 110 may receive an exercise plan allocated to the patient account, such that the patient U performs a rehabilitation exercise in accordance with the exercise plan allocated to the patient. Further, the communication unit 110 may, based on the rehabilitation exercise of the patient according to the exercise plan being performed, receive evaluation data of the patient for the exercise plan. Further, the communication unit 110 may receive and provide an updated personalized exercise plan to the user terminal 10 based on the evaluation data of the patient.

The storage unit 120, which may also be referred to as a database (DB), may be configured to store exercise plan history information, including an exercise plan allocated to a patient, the patient's evaluation data for the exercise plan, and an updated exercise plan based on the evaluation data.

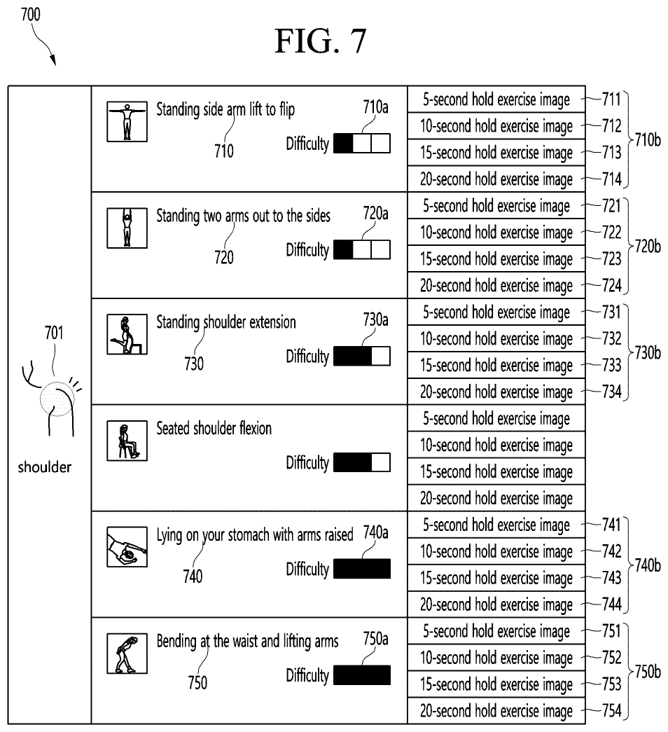

Further, the storage unit 120 may be configured to store, for each of the rehabilitation sites (e.g., shoulder, elbow, wrist & hand, hip joint & pelvis, knee, ankle & foot, neck, back, waist, abdomen) for indication therapy, matching information 700 with an exercise motion, difficulty information on the exercise motion, and an exercise image matched (see FIG. 7).

Further, the rehabilitation therapy provision system 100 according to the present invention may use data stored in an external storage separately from the storage unit 120, and this external storage may also be referred to as a “database.”

Meanwhile, the control unit 130 may control an overall operation of the rehabilitation therapy provision system 100 according to the present invention.

The control unit 130 may control a page (or a service page 300) for providing the musculoskeletal rehabilitation therapy service to be output through a display unit (or a touch screen) provided on the user terminal 10, as illustrated in FIG. 1. The page 300 may be output on the user terminal 10 through an application or webpage that is installed on the user terminal 10.

The page 300 is a page associated with the rehabilitation therapy provision system 100 according to the present invention, and is configured to be controlled by the rehabilitation therapy provision system 100 according to the present invention.

Further, when the page 300 is provided in the form of an application, the page 300 may be controlled by a central processing unit (CPU) of the user terminal 10 on which the application is installed. In this case, the CPU of the user terminal 10 may provide services related to musculoskeletal rehabilitation therapy to the patient based on information provided by the musculoskeletal rehabilitation therapy provision system 100 according to the present invention.

In the above, the constituent elements of the rehabilitation therapy provision system 100 according to the present invention have been described. Hereinafter, a method of providing a personalized musculoskeletal rehabilitation therapy service to a patient based on the constituent elements described above will be described.

As illustrated in FIG. 3, in the method of providing rehabilitation therapy according to the present invention, a process of determining an exercise plan to be provided to a patient may proceed based on prescription information including the exercise plan being allocated to the patient from the doctor terminal (S310).

In the present invention, as illustrated in FIG. 2B, an exercise prescription page (or exercise assignment page, 21) including a prescription function related to a rehabilitation exercise for a musculoskeletal patient may be provided on the doctor terminal 20 logged in with the doctor account.

Control of the exercise prescription page 21 may be performed by a server built to perform a service of providing rehabilitation therapy. The rehabilitation therapy provision system 100 according to the present invention may exist within the server, or as a separate system 100 from the server and communicatively connected thereto. That is, in the present invention, a physical space in which a process of providing a musculoskeletal rehabilitation therapy service is performed may not be distinguished, but the process may be described as being performed in the rehabilitation exercise therapy provision system 100. Further, the process of providing a musculoskeletal rehabilitation therapy service may be described as being performed by the communication unit 110, the storage unit 120, and the control unit 130 according to the present invention.

Meanwhile, the control unit 130 may provide, on the doctor terminal 20, the exercise prescription page 21 for each patient account, such that a prescription may be made for a specific patient U account among the patient accounts matched to the doctor account.

For example, in the present invention, assume that a specific doctor D account is matched with a first patient account (e.g., patient account “Jinkyu Sung”) and a second patient account (e.g., patient account “Sohee Kim”). The control unit 130 may provide, on the doctor terminal 20, an exercise prescription page corresponding to the first patient account (e.g., patient account “Jinkyu Sung”) based on an exercise prescription request for the first patient account being received from the doctor terminal 20.

The control unit 130 may allocate, based on a user selection (or user input) made on the exercise prescription page corresponding to the specific patient, prescription information for the specific patient from the doctor terminal 20.

The prescription information may include a variety of information related to the provision of a rehabilitation exercise plan to a musculoskeletal patient. For example, prescription information may include at least one of i) information on an indication (e.g., information on a pain disease), ii) information on rehabilitation site for a musculoskeletal patient (e.g. shoulder, elbow, wrist & hand, hip joint & pelvis, knee, ankle & foot, neck, back, waist, abdomen), iii) information on a rehabilitation period (e.g. 1 week, 4 weeks, or 8 weeks), iv) an rehabilitation exercise item that a musculoskeletal patient should exercise for rehabilitation (e.g., “Calf stretch against wall”, “Seated rolling ball sole massage”), v) difficulty information on the rehabilitation exercise item, vi) information on duration of the rehabilitation exercise item (e.g., hold for 10 seconds), vii) information on the number of repetitions of the rehabilitation exercise item (e.g., 5 repetitions), viii) information on a repetition set of the rehabilitation exercise item, ix) caution information (e.g., “Please after the exercise, apply an ice pack”), or x) information on an exercise tool (see FIG. 4).

Further, the prescription information may further include information on a digital remedy. As illustrated in FIG. 2B, the exercise prescription page 21 may include items 22, 23, and 24 representing digital remedies corresponding to each of different indications (e.g., patellofemoral pain syndrome, patellofemoral arthritis, chronic low back pain, etc.). In the present invention, a specific digital remedy may be selected from the doctor terminal 20 according to an indication of a patient. In the present invention, the digital remedy may also be understood as an “exercise plan” for an indication corresponding to the digital remedy. The storage unit 120 according to the present invention stores a plurality of different exercise plans, each of which may correspond to a digital remedy matched to a different indication. That is, in the present invention, the digital remedy may mean an exercise plan for an indication therapy. Accordingly, when a digital remedy item for a specific indication therapy (e.g., a digital remedy item for patellofemoral pain syndrome therapy, 22) is selected on the exercise prescription page 21, it may mean that an exercise plan for the patellofemoral pain syndrome therapy is selected as a prescription for the patient.

Here, the term “exercise item” may be understood as an exercise motion or an exercise type, and the terms “exercise item,” “exercise motion,” and “exercise type” may be used interchangeably in the present invention.

Meanwhile, the control unit 130 may, based on the prescription information for the patient being allocated from the doctor terminal 20, select (or specify) an exercise plan to be provided to the patient. That is, as described above, the control unit 130 may select an exercise plan corresponding to the digital remedy selected from the doctor terminal 20 as an exercise plan to be provided to the patient.

Meanwhile, an exercise plan corresponding to each of the digital remedies may include at least one exercise item assigned to each of a plurality of days or a plurality of weeks constituting the rehabilitation period. That is, an exercise plan corresponding to a digital remedy may include predetermined exercise items for indication therapy according to the corresponding digital remedy. The exercise items constituting each exercise plan may, based on an expert, a group of experts, or an artificial intelligence algorithm, be exercise items that have been selected as effective for an indication therapy targeted by a digital remedy according to each exercise plan. The rehabilitation period may be preset and exist within the system 100, or may exist to be included in the prescription information received from the doctor terminal 20. Hereinafter, for convenience of description, an example in which a rehabilitation period of “8 weeks” is set will be described.

The exercise plan may be configured with at least one different rehabilitation exercise item for each of the plurality of days constituting the rehabilitation period. For example, a first day of the exercise plan may be configured with a first exercise item and a second exercise item, and a second day may be configured with the first exercise item and a third exercise item.

Further, the exercise plan may be configured with at least one different rehabilitation exercise item for each of the plurality of weeks constituting the rehabilitation period. In this case, in the exercise plan, a plurality of days in the same week (e.g., Monday to Sunday) may include the same rehabilitation exercise item. For example, a first week (Monday to Sunday of week 1) of the exercise plan may be configured with the first exercise item and the second exercise item, and a second week (Monday to Sunday of week 2) may be configured with the first exercise item and the third exercise item.

Meanwhile, the control unit 130 may allocate, to the patient account, a cognitive behavioral therapy plan 220 that proceeds in conjunction with an exercise plan 210 during a preset rehabilitation period such that cognitive behavioral therapy is provided in conjunction with rehabilitation therapy for a musculoskeletal patient.

For example, as illustrated in FIG. 4, the control unit 130 may allocate the exercise plan 210 and the cognitive behavioral therapy plan 220 on a weekly basis for each of the plurality of therapy weeks that constitute the rehabilitation period. The control unit 130 may, in a first week rehabilitation period, proceed with an exercise plan 211 and a cognitive behavioral therapy plan 221 allocated for the first week in conjunction with each other, and in a second week rehabilitation period, proceed with an exercise plan 212 and a cognitive behavioral therapy plan 222 allocated for the second week in conjunction with each other. More details on the cognitive behavioral therapy will be described below.

As described above, the control unit 130 may set the cognitive behavioral therapy plan 220 to correspond to the preset rehabilitation period corresponding to the exercise plan 210. For example, when the preset rehabilitation period corresponding to the exercise plan 210 is eight weeks, the therapy period according to the cognitive behavioral therapy plan 220 may also be set to eight weeks. In contrast, when the preset rehabilitation period corresponding to the exercise plan 210 is six weeks, the therapy period according to the cognitive behavioral therapy plan 220 may also be set to six weeks. Meanwhile, the control unit 130 may also configure the preset rehabilitation period corresponding to the exercise plan 210 and the therapy period according to the cognitive behavioral therapy plan 220 independently of each other, depending on a situation. For example, the rehabilitation period according to the exercise plan 210 may be set to 6 weeks, and the therapy period according to the cognitive behavioral therapy plan 220 may be set to 8 weeks.

Meanwhile, in the present invention, a process in which the application is executed may proceed on the user terminal to which the patient account is logged in (S320, see FIG. 3).

The control unit 130 may, based on the application being executed, identify whether the exercise plan allocated to the patient account is activated. The exercise plan allocated to the patient account may be an exercise plan corresponding to the digital remedy selected on the doctor terminal 20, as described above.

Here, an “exercise plan activation” may be understood as a state of being able to provide the rehabilitation exercise service according to the exercise plan.

The control unit 130 may, based on the application being executed, control the exercise plan to be activated when the exercise plan allocated to the patient account is in an inactivated state.

For example, the control unit 130 may, based on the patient account being logged in through the executed application, control the exercise plan allocated to the patient account to be activated. In another example, the control unit 130 may provide, through the executed application, an icon associated with the activation of the exercise plan allocated to the patient account (e.g., “Mr. Jisuk Kim, do you want to start rehabilitation exercise?”) on the screen of the user terminal 10. Further, the control unit 130 may, based on the icon being selected, control the exercise plan allocated to the patient account to be activated.

Meanwhile, as illustrated in FIG. 2C, the patient may identify the digital remedy prescribed to the patient through the executed application. Information 31 and 32 on the digital remedy prescribed to the patient may be provided on a page 30 of the application. Meanwhile, when a plurality of digital remedies are selected for a patient, the patient may select the digital remedy for which the patient desires to perform rehabilitation therapy. In this case, the control unit 130 may provide the exercise plan matched to the selected digital remedy to the user terminal 10.

Meanwhile, the control unit 130 may, based on the exercise plan allocated to the patient account being activated, count the rehabilitation period matched to the exercise plan.

In the present invention, a day on which the counting of the rehabilitation period begins may be described to be referred to as a “start date” or “reference date” of the rehabilitation exercise. For example, in the present invention, the reference date may be counted as a first day and a day after the reference date as a second day. In still another example, in the present invention, seven days including the reference date may be described to be referred to as a first week, and seven days following the first week as a second week.

Meanwhile, as illustrated in (a) of FIG. 5A, the control unit 130 may, based on the application being executed, provide an initial screen page 300 on the user terminal 10.

The initial screen page 300 may be configured to include at least one of a plurality of first menu items to fourth menu items 310 to 340 associated with providing different services.

The first menu item 310 may be configured such that rehabilitation exercise service according to the exercise plan allocated to the patient account is associated with a rehabilitation exercise information page 600.

As illustrated in (b) of FIG. 5A, the control unit 130 may, based on the first menu item 310 being selected on the user terminal 10, provide, on the user terminal 10, the rehabilitation exercise information page 600 including an exercise list 610 according to the exercise plan. Accordingly, in the present invention, the first menu item 310 may be understood to be associated with a function of accessing the exercise list according to the exercise plan.

The second menu item 320 may be configured to be associated with a function of accessing evaluation pages (see reference numerals 710 to 730 in FIG. 5B) that provides an evaluation service for the exercise plan allocated to the patient account. The control unit 130 may, based on the degree of performance of the patient's rehabilitation exercise (or the degree of playback of the exercise image) satisfying a preset standard, control the second menu item 310 to be activated. In contrast, the control unit 130 may be configured to limit (i.e., inactivate) the provision of the evaluation pages 710 or 730 on the user terminal 10, even if the second menu item 320 is selected on the user terminal 10, when the degree of performance of the patient's rehabilitation exercise (or the degree of playback of the exercise image) does not satisfy the preset standard.

The third menu item 330 may be configured to be associated with a cognitive behavioral therapy page (see reference numeral 800 in FIG. 8A) that provides a cognitive behavioral therapy plan allocated to the patient account. More details on the cognitive behavioral therapy plan will be described below.

Further, the fourth menu item 340 may be configured to be associated with a functional evaluation page (or motion analysis page, see reference numeral 900 in FIG. 9) that performs a functional evaluation of the patient's specific motion. In the present invention, an artificial intelligence model trained with training data may be used to perform an analysis of posture and motion for a specific exercise motion of a patient from an exercise image of the patient. Accordingly, in the present invention, the functional evaluation that evaluates the patient's posture and motion for an exercise item may be referred to as “artificial intelligence (AI) functional evaluation”.

Meanwhile, in the present invention, a patient may be induced to perform the AI functional evaluation of a specific motion in order to identify a progress of the patient's remission (or an effect of a rehabilitation exercise) according to the rehabilitation exercise.

The control unit 130 may control such that the AI functional evaluation of the specific motion is performed every preset day interval (e.g., “two weeks”) of the preset rehabilitation period (e.g., “eight weeks”) to which the exercise plan is allocated, and the fourth menu item 340 is included on the initial screen page 300 on a specific day according to the preset day interval.

As described above, in the present invention, various services provided in the present invention may be provided on the user terminal 10 based on one of the plurality of menu items 310 to 340 constituting the initial screen page 300 being selected.

Meanwhile, in the present invention, a process of providing an exercise list according to the exercise plan to a user terminal on which the application is executed may be performed (S330, see FIG. 3).

The control unit 130 may, based on the first menu item 310 being selected on the initial screen page 300, provide the user terminal 10 with the rehabilitation exercise information page 600 including the exercise list 610.

As illustrated in (b) of FIG. 5A, the exercise list 610 may include a plurality of rehabilitation exercise items 611 to 616 that are allocated to a specific day on which the exercise list 610 is provided, based on the reference date on which the counting of the rehabilitation period has begun. For example, when a patient's day of rehabilitation exercise performance corresponds to “day 23” based on the reference date, exercise list 610 may include the exercise items 611 to 616 that are allocated to “day 23” of the plurality of days constituting the exercise plan matched to the patient account.

Further, the control unit 130 may, based on the exercise plan allocated to the patient account, place a specific exercise item on the exercise list 610 repeatedly by the number of sets. For example, when three sets of “Leg upright 1” exercise item are allocated in the exercise plan, the control unit 130 may place three exercise items 611, 612, and 613 on the exercise list 610, corresponding to the three leg upright Is.

Further, the control unit 130 may, based on the exercise plan allocated to the patient account, sequentially place the plurality of exercise items 611 to 616 on the exercise list 610. The control unit 130 may place a first exercise item 611 to 613 corresponding to a first priority exercise in sequence at the top of the exercise list 610, a second exercise item 614 corresponding to the next exercise in sequence at the bottom of the first exercise item.

Meanwhile, in the present invention, a process of playing an exercise image corresponding to each of the plurality of exercise items according to the plurality of exercise items constituting the exercise list may be performed on the user terminal (S340, see FIG. 3).

The control unit 130 may, based on the patient's request to start the rehabilitation exercise, sequentially play an exercise image corresponding to each of the exercise items 611 to 616 on the user terminal 10 according to the sequence of the exercise items 611 to 616 placed in the exercise list 610. For example, as illustrated in (b) of FIG. 5A, the control unit 130 may, based on a user selection of a “start exercise” icon included on the rehabilitation exercise information page 600, play an exercise image corresponding to each of the plurality of exercise items 611 to 616 on the user terminal 10.

As illustrated in FIG. 7, there may be exercise images 710b, 720b, 730b, and 740b corresponding to each of the exercise items in the storage unit 120. The control unit 130 may load an exercise image corresponding to the exercise item from the storage unit 120 and display the exercise image on the user terminal 10.

Meanwhile, in the present invention, based on a degree of playback of the exercise image satisfying the preset standard, a process of providing an evaluation page for performing an evaluation related to the exercise item may proceed (S350, see FIG. 3).

As described above, in the present invention, feedback on the exercise plan may be received from the patient, and the exercise plan may be updated to reflect the feedback so that a personalized exercise plan is provided for each patient.

The control unit 130 may monitor whether the patient performs the rehabilitation exercise in order to receive feedback on the exercise plan from the patient who actually performs the rehabilitation exercise according to the exercise plan allocated to the patient account. Further, when judging whether the patient has actually performed the rehabilitation exercise as a result of the monitoring, the control unit 130 may provide the evaluation pages 710 to 730 for receiving evaluation information on the rehabilitation exercise performed by the patient.

Specifically, when the exercise images corresponding to the plurality of exercise items 611 to 616 included in the exercise list 610 are played on the user terminal 10, the control unit 130 may monitor the degree of playback of the exercise images to judge the patient's rehabilitation exercise performance rate (or the degree of rehabilitation exercise performance). Further, the control unit 130 may, based on the degree of playback of the exercise image satisfying the preset standard, provide the evaluation pages 710 to 730 on the user terminal 10.

The control unit 130 may perform to determine whether to provide the degree of playback of the exercise image and the evaluation page based on at least one of a playback time of the exercise image or the number of exercise items corresponding to the played exercise images.

The control unit 130 may determine whether to provide the evaluation pages 710 to 730 based on whether the playback time of the exercise image exceeds a preset standard playback time.

The control unit 130 may determine to provide the evaluation pages 710 to 730 (or activate the evaluation pages) when the playback time of the exercise image exceeds the preset standard playback time, and may determine not to provide the evaluation pages 710 to 730 (or deactivate the evaluation pages) when the playback time of the exercise image does not exceed the preset standard playback time.

In this case, the control unit 130 may set a standard playback time based on which the evaluation pages 710, 720, and 730 are provided differently based on an overall playback time (or total playback time) of the exercise images corresponding to the plurality of exercise items 611 to 616 included in the exercise list.

Specifically, the control unit 130 may set a time corresponding to a predetermined range (or proportion) of the total playback time as the standard playback time, such that the standard playback time is proportional to the overall playback time (or total playback time) of the exercise images corresponding to the plurality of exercise items 611 to 616 included in the exercise list.

Meanwhile, the control unit 130 may determine to provide whether to provide the evaluation pages 710 to 730 based on whether the number of exercise items corresponding to the played exercise images (hereinafter referred to as the “number of played exercise items”) exceeds a preset standard number of plays.

The control unit 130 may determine to provide the evaluation page 710 to 730 (or activate the evaluation page) when the number of played exercise items exceeds the preset standard number of plays, and determine not to provide the evaluation page 710 to 730 (or deactivate the evaluation page) when the number of played exercise items does not exceed the preset standard number of plays.

In this case, the control unit 130 may set the standard number of plays based on which the evaluation pages 710 to 730 are provided differently based on the overall number (or total number) of the plurality of exercise items 611 to 616 included in the exercise list.

Specifically, the control unit 130 may set a number corresponding to a predetermined range (or proportion) of the overall number of the plurality of exercise items 611 to 616 included in the exercise list as the standard number of plays, such that the standard number of plays is proportional to the overall number (or total number) of the plurality of exercise items 611 to 616 included in the exercise list.

That is, the control unit 130 may, based on the plurality of exercise items allocated to a specific day, set a standard (standard playback time or standard number of plays) for providing the evaluation page on a specific day differently for each specific day corresponding to a day when the patient performs the rehabilitation exercise.

Meanwhile, the control unit 130 may control a second area 320 associated with the evaluation page to be activated in the initial screen page 300 when the evaluation page 710 to 730 is determined to be provided (see (a) of FIG. 5A). For example, the control unit 130 may, based on the evaluation page being determined to be provided, display the second area 320 on the initial screen page 300.

Further, as illustrated in (c) of FIG. 5A, a pop-up 602 associated with the evaluation page may be provided on the user terminal 10 based on the evaluation page 710 to 730 being determined to be provided. For example, the control unit 130 may, based on the playback of the images corresponding to the plurality of exercise items 611 to 616 included in the exercise list 610 having been completed, provide the pop-up 620 on the user terminal 10. Further, the control unit 130 may, based on an icon (e.g., “Go to self-check”, 620a) included in the pop-up 620 being selected, provide the evaluation pages 710 to 730 on the user terminal 10.

Meanwhile, the evaluation pages 710 to 730 may be configured with a plurality of evaluation areas, such that an evaluation is performed for each of a plurality of evaluation categories for the plurality of exercise items allocated to a specific day.

Here, the term “evaluation category” refers to a category that is subject to evaluation for an exercise plan so that a personalized rehabilitation exercise plan may be provided in consideration of a state of a musculoskeletal patient, and may include, for example, at least one of exercise difficulty for exercise items, an exercise item of high difficulty (difficult exercise item), or pain after a rehabilitation exercise.

The control unit 130 may configure the evaluation page to include an evaluation area corresponding to each of the plurality of evaluation categories. In this case, the plurality of evaluation areas may be disposed on the same page, or on each of the different evaluation pages 710, 720, and 730, as illustrated in FIG. 5B. Accordingly, the terms “evaluation page” and “evaluation area” may be used interchangeably in the present invention. In the present invention, the “evaluation area” may also be described by giving the same reference numerals “710 to 730” as the “evaluation page”.

The control unit 130 may configure the first evaluation area 710 such that the exercise difficulty for the plurality of exercise items allocated to a specific day may be evaluated.

As illustrated in (a) of FIG. 5B, the control unit 130 may, based on question information inquiring about the exercise difficulty (e.g., “How was your exercise intensity today?”) and evaluation information on the exercise difficulty, allow guide information guiding that the exercise plan is updated (e.g., “Exercise organization and difficulty are adjusted based on your input.”), and an evaluation information input area for receiving the evaluation information on the exercise difficulty (hereinafter referred to as a “first input area,” 710a) to be included on the first evaluation area 710.

The control unit 130 may allow a slider corresponding to a preset exercise difficulty scale to be included on the first input area 710a. Further, the control unit 130 may control, in conjunction with the slider, such that an emotion graphic object and emotion information corresponding to the exercise difficulty specified through the slider are output on the first evaluation area 710.

For example, the control unit 130 may output a “smile” emotion graphic object and emotion information of “I feel like I haven't exercised” on the first evaluation area 710 when the exercise difficulty specified by the slider is “lowest difficulty” (e.g., 0 points). In another example, the control unit 130 may output a “frown” emotion graphic object and emotion information of “I had difficulty following the exercise” on the first evaluation area 710 when the exercise difficulty specified by the slider is “highest difficulty” (e.g., 9 points).

The patient may intuitively recognize, in the first evaluation area 710, a range corresponding to a lowest to highest score of the exercise difficulty through the slider. Further, the patient may input the evaluation information while visually confirming the difficulty of the rehabilitation exercise through the graphic object and emotional information output in conjunction with the slider.

Further, the control unit 130 may, based on the evaluation information being input to the first evaluation area 710, receive the evaluation information on the exercise difficulty from the first evaluation area 710.

Meanwhile, as illustrated in (b) of FIG. 5B, the control unit 130 may, based on question information inquiring about an exercise item of an exercise of high difficulty (e.g., “Please tell us about your painful or uncomfortable motions.”) and evaluation information on the exercise item of the exercise of high difficulty, allow the guide information guiding that the exercise plan is updated (e.g., “Exercise organization and difficulty are adjusted based on your input.”) and an input area for receiving a selection of the exercise item of high difficulty (hereinafter referred to as a “second input area”) to be included on the second evaluation area 720.

Further, the control unit 130 may control such that the second input area of the second evaluation area 720 includes a plurality of check items 721a to 726a for receiving a user selection for an exercise item of higher difficulty (a difficult exercise item) as input among the plurality of exercise items 611 to 616 allocated to a specific day (e.g., “Day 23”).

The control unit 130 may correspond one of the plurality of check items (e.g., “There was no uncomfortable motion:)”, 721a) to an option to not select any of the plurality of exercise items 611 to 616 allocated to a specific day (e.g., “Day 23”) as an exercise item of high difficulty. Further, the control unit 130 may correspond the plurality of exercise items 611 to 616 allocated to a specific day (e.g., “day 23”) to each of the remaining check items 722a to 726a, and display information on the plurality of exercise items 611 to 616 (e.g., a thumbnail of an image corresponding to an exercise item or a title of the exercise item).

The control unit 130 may receive the evaluation information on the exercise item of high difficulty through a user selection of a check box included in the second evaluation area 720.

As illustrated in (c) of FIG. 5B, the control unit 130 may, based on question information inquiring about an exercise pain (e.g., “How much pain do you feel after the rehabilitation exercise?”) and evaluation information on the exercise pain, allow the guide information guiding that the exercise plan is updated (e.g., “Exercise organization and difficulty are adjusted based on your input.”), and an evaluation information input area for receiving the evaluation information on the exercise pain (referred to as a “third input area,” 730a) to be included on the third evaluation area 730. The third evaluation area 730 may be configured to be identical to the first evaluation area 710 except for the question information. In this regard, a detailed description will be omitted.

The control unit 130 may receive the evaluation information on the exercise pain through the third evaluation area 730.

Meanwhile, in the present invention, an updated exercise plan may be provided to the patient based on the patient's evaluation information on an exercise plan, through changes such as excluding or adding at least some of the exercise items constituting the exercise plan or the difficulty of the exercise plan. Hereinafter, a method of updating an exercise plan based on evaluation information will be described in detail.

The control unit 130 may, based on evaluation information on an exercise item of high difficulty received from the second evaluation area 720 (hereinafter referred to as “second evaluation information”), update an exercise plan through changes (or replacements) in exercise items, such as excluding at least some of the exercise items included in the exercise plan allocated to the patient account, or adding new exercise items.

In this case, the exercise items subject to exclusion or replacement may correspond to exercise items allocated on or after the next day (“Day 24”) following a specific day (“Day 23”) of a plurality of days constituting a rehabilitation period (e.g., Week “8”). That is, the control unit 130 may, based on evaluation information for a specific day (in particular, the second evaluation information), exclude or replace at least some of the exercise items from the next day of the specific day.

The control unit 130 may exclude a specific item selected as an exercise of high difficulty among the exercise items allocated for a specific day from an exercise plan on or after the next day of the specific day. Further, the control unit 130 may add (include) a different exercise item in replacement of the excluded exercise item. In contrast, the control unit 130 may exclude a specific item selected as an exercise of high difficulty, among the exercise items allocated to a specific day, from an exercise plan for a different specific day that is next performed after the specific day, which may be the next day after the specific day or a different day. In this case, the control unit 130 may also add (include) a different exercise item in replacement of the excluded exercise item.

More specifically, the control unit 130 may replace a specific exercise item selected as an exercise of high difficulty in the second evaluation area with a different exercise item having the same difficulty level among the exercise items matched to the same rehabilitation site. Here, the different exercise item may be an exercise that has the same or similar efficacy (e.g., rehabilitation efficacy) as the selected specific exercise item. Further, the different exercise item may be an exercise that causes less pain than the selected specific exercise item. In this case, the efficacy and degree of pain for each exercise item may be judged based on information matched to each exercise item.

As illustrated in FIG. 7, a plurality of exercise items 710 to 750 may exist in storage unit 120, matched in the same group, by a rehabilitation site (e.g., shoulder, elbow, wrist & hand, hip joint & pelvis, knee, ankle & foot, neck, back, lower back, abdomen) or by an indication (e.g., patellofemoral pain syndrome, patellofemoral arthritis, chronic low back pain, etc.). The exercise items in the same group, matched by a rehabilitation site, may correspond to exercise items for rehabilitation of a specific rehabilitation site.

Further, the matching information 700 may include difficulty level information (e.g., “difficulty level 1 to difficulty level 3”, 710a to 750a) for each of the exercise items matched to a specific rehabilitation site (e.g., “shoulder”, 701) and different exercise hold time information (or exercise repetition count information) for each of the exercise items. Further, a plurality of exercise images 711 to 714 corresponding to different exercise hold time information (or exercise repetition count information) may exist to be matched for each specific exercise item (e.g., “Standing side arm lift to flip”, 710) in the matching information 700.

For example, the exercise hold time information may include one of “hold for 5 seconds”, “hold for 10 seconds”, “hold for 15 seconds”, or “hold for 20 seconds”, and the exercise repetition count information may include one of “5 repetitions”, “10 repetitions”, “15 repetitions”, or “20 repetitions”. Hereinafter, for convenience of description, an exercise motion hold time will be described as an example.

As illustrated in (a) of FIG. 5C, assume that the plurality of exercise items 611 to 616 are allocated to a specific day (“Day 23”), of which the first exercise item (e.g., “Standing side arm lift to flip”, 611) is selected as an exercise of high difficulty.

As illustrated in FIG. 7, different exercise items 720 to 750 may exist in the matching information 700 that are matched to the same rehabilitation site (e.g., “shoulder” 701) as the specific exercise item 710 selected as an exercise of high difficulty. The control unit 130 may specify a different exercise item (e.g., “stand and raise both arms to the side”, 720) having the same difficulty level information (e.g., level 1, 720a) as the difficulty level information (e.g., level 1, 710a) of the specific exercise item 710 as an alternate exercise item among the different exercise items 720 to 750.

The control unit 130 may update the exercise plan allocated to the patient account, beginning on a day after a specific day has elapsed (e.g., “Day 24”), by excluding the exercise item selected as an exercise of high difficulty and adding (or newly including) the alternative exercise item.

Further, as illustrated in (b) of FIG. 5C, the control unit 130 may provide the exercise list 620, including the exercise items 621 to 626 according to the updated exercise plan, to the user terminal 10, beginning on the day after the specific day has elapsed (e.g., “day 24”).

Meanwhile, the control unit 130 may set an exercise intensity of the alternative exercise item to be the same as an exercise intensity of the specific exercise item selected as an exercise of high difficulty.

Here, the term “exercise intensity” may mean exercise hold time information (or exercise repetition count information) and the number of sets.

For example, the control unit 130 may set an exercise intensity of the alternate exercise item 621 to “1 set of 10 second holds” when an exercise intensity of the exercise item 611 selected as an exercise of high difficulty is “1 set of 10 second holds”.

Meanwhile, the control unit 130 may, based on the evaluation information received from one of the first evaluation area 710 or the third evaluation area 730, adjust (or change) the difficulty of each of the exercise items included in the exercise plan allocated to the patient account to update the exercise plan.

In this case, the exercise items subject to the difficulty adjustment may mean exercise items allocated on or after the next day of a specific day (“Day 23”) of the plurality of days constituting the rehabilitation period (e.g., week “8”). That is, the control unit 130 may, based on the evaluation information on a specific day, change the difficulty of the exercise items from the next day of the specific day.

The control unit 130 may, based on evaluation information received from one of the exercise difficulty evaluation information received from the first evaluation area 710 (hereinafter referred to as first evaluation information) and the exercise pain evaluation information received from the third evaluation area 730 (hereinafter referred to as third evaluation information), determine one of difficulty down, difficulty hold, or difficulty up.

The control unit 130 may, based on one of the first evaluation information or the third evaluation information, determine to change the difficulty of the exercise items constituting the exercise plan. For example, the control unit 130 may determine to change the difficulty of the exercise items using the first evaluation information. In addition, the control unit 130 may determine to change the difficulty of the exercise items using the third evaluation information. In addition, the control unit 130 may determine to change the difficulty of the exercise items using an evaluation score calculated as a sum or average of the first evaluation information and the third evaluation information. Hereinafter, for convenience of description, an example of determining to change the difficulty of the exercise items based on the exercise difficulty evaluation information will be described.

The control unit 130 may identify whether the exercise difficulty evaluation information on the exercise items allocated on a specific day (e.g., “Day 23”) corresponds to one of respective difficulty change standards, such as difficulty down, difficulty hold, difficulty up, and exercise update, to determine whether to change the difficulty of the exercise items allocated on or after the next day of the specific day.

Specifically, the control unit 130 may determine the difficulty down when the exercise difficulty evaluation information corresponds to a first difficulty change standard, determine the difficulty hold when the exercise difficulty evaluation information corresponds to a second difficulty change standard, and determine the difficulty up when the exercise difficulty evaluation information corresponds to a third difficulty change standard.

For example, assume that the evaluation information on the exercise difficulty is a difficulty score on a scale of “0 to 9” (natural numbers). The control unit 130 may determine the difficulty down for the exercise items allocated on or after the next day of the specific day when the difficulty score for the exercise items allocated on the specific day is “9”, “8”, “7”, or “6”. Further, the control unit 130 may determine the difficulty hold for the exercise items allocated on or after the next day of the specific day when the difficulty score for the exercise items allocated on the specific day is “5”, “4”, or “3”. Further, the control unit 130 may determine the difficulty up for the exercise items allocated on or after the next day of the specific day when the difficulty score for the exercise items allocated on the specific day is “2”, “1”, or “0”.

Meanwhile, when one of the difficulty down or the difficulty up of the exercise items allocated on or after the next day of a specific day is determined, the control unit 130 may change at least one of the number of times of holding the exercise motion (or the number of times of repeating the exercise motion) or the number of sets for each of the exercise items allocated on the specific day to change the exercise difficulty.

As illustrated in (a) of FIG. 5C, assume that the plurality of exercise items 611 to 616 are allocated for a specific day (“Day 23”). When the difficulty change is determined based on the exercise difficulty evaluation information on the exercise items 611 to 616 allocated to a specific day (“Day 23”), the control unit 130 may change the difficulty of the exercise items 611 to 616. Hereinafter, for convenience of description, a method of changing the difficulty of a specific exercise item (e.g., “Standing shoulder extension”, 612) will be described as an example. The method of changing the difficulty of the specific exercise item 612 may be applied equally to other exercise items.

The control unit 130 may, with reference to the matching information 700 existing in the storage unit 120, change at least one of the exercise motion hold time (or the number of times of repeating the exercise motion) or the number of sets of a specific exercise item (standing shoulder extension, 730), to change the difficulty of the specific exercise item.

A minimum exercise motion hold time (e.g., “5 seconds”), a maximum exercise motion hold time (e.g., “20 seconds”), a minimum number of sets (e.g., “1 set”), and a maximum number of sets (e.g., “3 sets”) may exist to be matched for each exercise item. Meanwhile, in the present invention, for convenience of description, the “exercise motion hold time” is described as an example, but depending on a type of exercise item, may be described as the “number of times of repeating the exercise motion”.

The control unit 130 may, based on the determination of the difficulty up of the exercise items, increase the exercise motion hold time for the specific exercise item 730. For example, when the current exercise hold time of the specific exercise motion 730 is “5 seconds” 731, the control unit 130 may increase the exercise hold time of the specific exercise item 730 to “10 seconds” 732.

When the current exercise hold time of the specific exercise motion 730 is maximized, the control unit 130 may increase the current number of sets of the specific exercise item. For example, when the current exercise motion hold time of the specific exercise motion 730 is “20 seconds” and the current number of sets is “1 set”, the control unit 130 may increase the number of sets of the specific exercise item 730 to “2 sets”.

Meanwhile, when the current exercise hold time and the current number of sets of a specific exercise item are both maximized, the control unit 130 may replace the specific exercise item with a different exercise item having a difficulty level that is higher than the difficulty level of the specific exercise item. For example, when the specific exercise item 730 has a difficulty level of “2,” the control unit 130 may replace (or substitute) the specific exercise item 730 with a different exercise item 740 having a difficulty level of “3.” In this case, the control unit 130 may determine the exercise hold time and the number of sets of the different exercise item 740 as the maximum exercise hold time and the maximum number of sets matched to the different exercise item 740.

Meanwhile, the control unit 130 may, based on the determination of the difficulty down of the exercise items, decrease the exercise motion hold time for the specific exercise item 730. For example, when the current exercise hold time of the specific exercise motion 730 is “15 seconds” 733, the control unit 130 may decrease the exercise hold time of the specific exercise item 730 to “10 seconds” 732.

When the current exercise hold time of the specific exercise motion 730 is minimized, the control unit 130 may decrease the current number of sets of the specific exercise item. For example, when the current exercise motion hold time of the specific exercise motion 730 is “5 seconds” and the current number of sets is “2 set”, the control unit 130 may decrease the number of sets of the specific exercise item 730 to “1 sets”.

Meanwhile, when the current exercise hold time and the current number of sets of a specific exercise item are both minimized, the control unit 130 may replace the specific exercise item with a different exercise item having a difficulty level that is lower than the difficulty level of the specific exercise item. For example, when the specific exercise item 730 has a difficulty level of “2,” the control unit 130 may replace (or substitute) the specific exercise item 730 with a different exercise item 720 having a difficulty level of “1.” In this case, the control unit 130 may determine the exercise hold time and the number of sets of the different exercise item 730 as the maximum exercise hold time and the maximum number of sets matched to the different exercise item 730.

Meanwhile, the control unit 130 may, based on the evaluation information on the exercise items allocated on a specific day (e.g., “Day 23”), change at least one of exercise types and difficulty of the exercise items allocated on the next day after the specific day (e.g., “Day 23”) to update the exercise plan.

The control unit 130 may provide, on the user terminal 10, an exercise list that includes exercise items according to the updated exercise plan, beginning on the next day after a specific day.

Meanwhile, the control unit 130 may, based on the exercise plan being updated based on the patient's evaluation information, provide information guiding the exercise plan update to the user terminal 10.

An occasion and method for guiding the exercise plan update may vary. For example, the control unit 130 may guide an exercise plan update when the application is executed on the next day of a specific day. In another example, when the first menu item 310 is selected on the initial screen page 300 on the next day of a specific day, the control unit 130 may provide an exercise plan update guide page 600′ on the user terminal 10 before providing an exercise list according to the exercise items included in the updated exercise plan.

In this case, at least one of exercise plan update reason information and exercise plan update content information may be displayed on the exercise plan update guide page 600′.

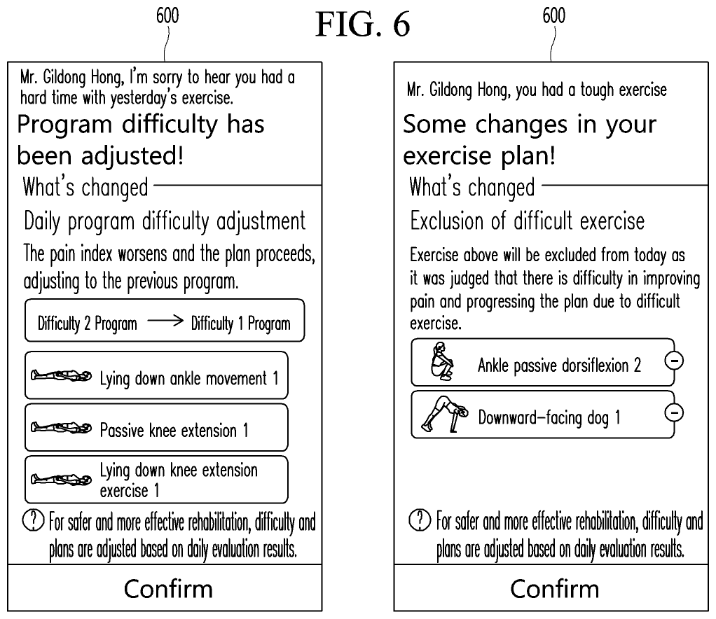

For example, as illustrated in FIG. 6A, based on the evaluation information on the exercise items allocated on a specific day and the difficulty of the exercise item allocated on the next day of the specific day being changed, the control unit 130 may display, on the exercise plan update guide page 600′, difficulty change reason information (e.g., “Mr. Gildong Hong, I'm sorry to hear you had a hard time with yesterday's exercise” or “The pain index has worsened and the plan is adjusted to the previously conducted program”) and difficulty change content information (e.g., “difficulty 2 program->difficulty 1 program”).

In another example, as illustrated in FIG. 6B, based on the evaluation information on the exercise items allocated for a specific day and at least some of the exercise items allocated for the next day of the specific day being excluded, the control unit 130 may display, on the exercise plan update guide page 600, exercise item type change reason information (e.g., “The corresponding exercise will be excluded from today as it was judged that there is difficulty in improving pain and progressing the plan due to difficult exercise.”) and exercise item change content information (e.g., “Ankle passive dorsiflexion 2 excluded”).

Meanwhile, the method and system 100 for providing musculoskeletal rehabilitation therapy according to the present invention may provide cognitive behavioral therapy, in conjunction with rehabilitative exercise therapy, to a patient suffering from pain due to a musculoskeletal disease.

In particular, in the present invention, it is possible to comprehensively provide cognitive behavioral therapy along with a progress of a rehabilitation exercise of a musculoskeletal patient by sequentially providing a plurality of programs that are different from each other during a rehabilitation period that is set for the rehabilitation exercise.

As described above, the cognitive behavioral therapy plan 220 may be set to correspond to the preset rehabilitation period corresponding to the exercise plan 210. For example, when the preset rehabilitation period corresponding to the exercise plan 210 is eight weeks, the therapy period according to the cognitive behavioral therapy plan 220 may also be set to eight weeks. In contrast, when the preset rehabilitation period corresponding to the exercise plan 210 is six weeks, the therapy period according to the cognitive behavioral therapy plan 220 may also be set to six weeks. Meanwhile, the control unit 130 may also configure the preset rehabilitation period corresponding to the exercise plan 210 and the therapy period according to the cognitive behavioral therapy plan 220 independently of each other, depending on a situation. For example, the rehabilitation period according to the exercise plan 210 may be set to 6 weeks, and the therapy period according to the cognitive behavioral therapy plan 220 may be set to 8 weeks.

Meanwhile, the rehabilitation therapy provision system 100 according to the present invention may also be referred to as the cognitive behavioral therapy provision system 100.

The present invention is directed to providing a cognitive behavioral therapy protocol personalized for a pain patient, in consideration of pain duration and degree of cognitive distortion for pain of the pain patient, and more particularly, to a method and system for providing cognitive behavioral therapy centered on a pain patient caused by a musculoskeletal disease.

For convenience of description, the present invention is described with a focus on “musculoskeletal diseases,” but is not necessarily limited thereto. That is, in the present invention, the cognitive behavioral therapy may include both cognitive behavioral therapy for a pain patient due to a musculoskeletal disease, as well as cognitive behavioral therapy for a pain patient due to a variety of diseases (e.g., cancer, diabetes, hypertension, etc.).

In the present invention, the term “pain patient” is used interchangeably with “patient” or “user” to refer to a patient experiencing pain due to a disease.

Meanwhile, in case of the pain patient, each patient may have various pain duration and degree of cognitive distortion for pain, even if the patient has the same disease (or illness). Accordingly, in the present invention, a personalized cognitive behavioral therapy protocol may be provided to a pain patient, reflecting the pain duration and degree of cognitive distortion for the patient's pain.

Meanwhile, the rehabilitation therapy provision system 100 according to the present invention may diagnose the pain duration and degree of cognitive distortion for the pain of the “patient” U using the user terminal 10, and provide a personalized cognitive behavioral therapy protocol in consideration thereof.

The term “cognitive distortion” means a cognitive error that leads to a false assumption or misconception about pain, surrounding circumstances, events, etc. and the term “degree of cognitive distortion” may be understood as a term that expresses how much a patient is experiencing cognitive distortion. In the present invention, the term “degree of cognitive distortion” may be used interchangeably with the term “cognitive distortion state” and may be quantified as a “cognitive distortion score”.

Meanwhile, as illustrated in (a) of FIG. 8A, the patient U may select the third menu item 330 on the initial screen page 300 to be provided with the cognitive behavioral therapy plan allocated to the patient account. As illustrated in (b) and (c) of FIG. 8A, the patient may manage his or her mental health state, such as diagnosing the pain duration and degree of cognitive distortion for his or her pain, and being provided with a consultation service according to a personalized cognitive behavioral therapy protocol, through the cognitive behavioral therapy page 800 provided by the rehabilitation therapy provision system 100 according to the present invention.

Hereinafter, a method of providing a personalized cognitive behavioral therapy protocol to a pain patient will be described.

Hereinafter, the cognitive behavioral therapy provision system 100 according to the present invention will be described according to an embodiment implemented as an application on the user terminal 10.

the cognitive behavioral therapy provision system 100 for a pain patient according to the present invention may be configured to include at least one of the communication unit 110, the storage unit 120, or the control unit 130. In this case, the cognitive behavioral therapy provision system 100 according to the present invention is not limited to the constituent elements described above, and may further include constituent elements that serve the same function as the devices described herein.

The communication unit 110 may include one or more therapy modules to enable wireless or wired communication between the cognitive behavioral therapy provision system 100 and the user terminal 10, or between the cognitive behavioral therapy provision system 100 and an external server. In addition, the communication unit 110 may include one or more communication modules that connect the cognitive behavioral therapy provision system 100 to one or more networks.

Specifically, the communication unit 110 may collect, through the user terminal 10, survey response data for diagnosing a patient's pain duration and degree of cognitive distortion from at least one source. In addition, the communication unit 110 may provide a personalized cognitive behavioral therapy service according to the pain duration and degree of cognitive distortion of the pain patient through the user terminal 10.

The storage unit 120, which may also be referred to as a database (DB), is configured to store various information (or data) related to the cognitive behavioral therapy of the pain patient, such as the collected survey response data, a diagnosis result according to the survey response data, and a cognitive behavioral therapy protocol according to the diagnosis result.

Further, the cognitive behavioral therapy provision system 100 according to the present invention may use data stored in an external storage separately from the storage unit 120, and this external storage may also be referred to as a “database.”

Meanwhile, the control unit 130 may control an overall operation of the cognitive behavioral therapy provision system 100 according to the present invention.

The control unit 130 may control a page (or a service page) for providing a cognitive behavioral therapy service to be output through a display unit (or a touch screen) provided on the user terminal 10, as illustrated in FIG. 1. The page may be output on the user terminal 10 through an application or webpage that is installed on the user terminal 10.

The page is a page associated with the cognitive behavioral therapy provision system 100 according to the present invention, and is configured to be controlled by the cognitive behavioral therapy provision system 100 according to the present invention.

Further, when the page is provided in the form of an application, the page may be controlled by a central processing unit (CPU) of the user terminal 10 on which the application is installed. In this case, the CPU of the user terminal 10 may provide services related to cognitive behavioral therapy to the pain patient based on information provided by the cognitive behavioral therapy provision system 100 according to the present invention.

In the above, the constituent elements of the cognitive behavioral therapy provision system 100 according to the present invention have been described. Hereinafter, a method of providing a personalized cognitive behavioral therapy protocol to a pain patient based on the constituent elements described above will be described.

As illustrated in FIG. 8B, a method of providing cognitive behavioral therapy according to the present invention may include a process of providing a plurality of survey data for diagnosing a state of a pain patient on the user terminal (S210).

The control unit 130 may control such that a page including a plurality of survey data is provided on the user terminal 10. In this case, the page including the plurality of survey data may be output through a touch screen (or a display) of the user terminal 10.

Here, the plurality of survey (or question, problem, statement, or test) data may include a survey for assessing a state on factors related to pain.

In the present invention, the factors related to pain may vary. For example, the factors related to pain may mean a variety of elements to assess a patient's state related to pain, such as degree of pain, pain duration, mental health, and physical health. However, it is obvious that the factors described above are just illustrative, and that the factors related to pain described in the present invention may mean any factors for evaluating the patient's state.

These factors related to pain may include validated survey data that are actually used in psychiatry to diagnose the patient's state. In addition, the control unit 130 may update the survey data frequently or periodically by downloading additional surveys for diagnosing the patient's state through an external server or website or the like.

Hereinafter, with reference to FIG. 8C, a user interface through which the survey data is provided is described. As illustrated in FIG. 8C, a page including a survey area 401 for displaying at least one survey of the plurality of surveys and a response area 402 for receiving a patient's response to the displayed survey may be output on the user terminal 10. In this case, the response area 420 may include response graphic objects 402a and 402b corresponding to each of a plurality of different responses to the surveys displayed in the survey area 401.

Accordingly, in response to any one of the displayed surveys, the pain patient may select a candidate response that is believed to be the most appropriate (or appropriate, or accurate) for the user's state among candidate responses output in the response area 402 as the survey response data.

Meanwhile, in the present invention, a process of receiving the survey response data for the plurality of survey data may be performed via the user terminal (S220, see FIG. 8B).

The control unit 130 may receive the survey response data from the user terminal 10 based on a user selection (or user input) for the response area 402 of the user terminal 10.

For example, as illustrated in FIG. 8C, based on one of the plurality of response graphic objects 402a and 402b corresponding to a first survey of the pain patient's pain duration (e.g., “When did Ms. Hyeryun Jang's knee pain start?”) being selected, a response (e.g., “1 week ago”) corresponding to the selected response graphic object 402a may be received as survey response data for the first survey. Meanwhile, the first survey may include a plurality of surveys. In this case, the user terminal 10 may be provided with a plurality of surveys necessary to detect state information on the patient's pain duration. Further, a response to the plurality of surveys may be received from the user terminal as the survey response data.

In another example, as illustrated in FIG. 8C, based on any one of the plurality of response graphic objects corresponding to a second survey related to a patient's degree of cognitive distortion being selected, a response (e.g., “3”) corresponding to the selected response graphic object may be received as survey response data for the second survey.

Meanwhile, the second survey may include a plurality of surveys. In this case, the user terminal 10 may be provided with a plurality of surveys necessary to detect state information on the pain patient's degree of cognitive distortion. Further, a response to the plurality of surveys may be received from the user terminal as the survey response data. An example of the second survey may include a survey related to at least one of pain catastrophizing scale or fear (or risk) avoidance beliefs, such as: i) I am always worried that the pain will not stop, ii) I feel like it is unbearable, iii) The pain is so painful that I think I will never get better, iv) My pain is caused by physical activity, v) Physical activity makes my pain worse, vi) My pain is caused by work or an accident at work, etc. Further, unlike the above, the plurality of survey data to diagnose the pain patient's state may further include a survey to judge depression, insomnia, etc.

Meanwhile, in the present invention, based on the survey response data, a process of detecting state information on the pain patient related to the pain duration and degree of cognitive distortion of the pain patient may be performed.

As illustrated in FIG. 8D, the control unit 130 may compare pain duration 450 extracted (obtained or detected) from the survey response data of the pain patient to a first reference (e.g., “3 months”, 450a) in order to detect the state information on the pain patient.

Here, the first reference 450a may be understood as a predefined reference period (e.g., “3 months”) for categorizing (or distinguishing) the pain duration (or the nature of the pain based on the pain duration) into one of “a first type of pain duration (e.g., acute pain, 451)” or “a second type of pain duration (e.g., chronic pain, 452)”.

When the pain duration 450 of the pain patient is shorter than the predefined reference period 450a (i.e., equal to or less than the predefined reference period) as a result of the comparison, the control unit 130 may categorize (distinguish, specify, or determine) the pain duration of the pain patient as a “first type of pain duration (e.g., acute pain, 451)”. In contrast, when the pain duration 450 of the pain patient is longer than the predefined reference period 450a (i.e., exceeds the predefined reference period), the control unit 130 may categorize the pain duration of the pain patient as a “second type of pain duration” (e.g., chronic pain, 452).

The control unit 130 may categorize the pain duration of the pain patient into one of acute pain or chronic pain, depending on whether the pain duration 450 exceeds the reference period 450a.

Further, the control unit 130 may compare a degree of cognitive distortion 460 extracted (obtained or detected) from the survey response data of the pain patient to a second reference 460a.

Here, the degree of cognitive distortion of the pain patient may be related to one of the pain catastrophizing scale or fear (or risk) avoidance beliefs, as described above.

Here, the second reference 460a may be understood as a predefined reference score (e.g., “24 points”) for categorizing (or distinguishing) the degree of cognitive distortion of the pain patient into one of a “first type of degree of cognitive distortion (e.g., high cognitive distortion, 461)” and a “second type of degree of cognitive distortion (e.g., low cognitive distortion, 462).

The control unit 130 may calculate (derive or compute) a cognitive distortion score of the pain patient based on the survey response data to compare the degree of cognitive distortion 460 of the pain patient to the predefined reference score 460a.

A plurality of selection items on a survey to judge the degree of cognitive distortion (e.g., I am always worried that the pain will not stop) may each be matched with a different score (e.g., not at all: 0 point, a little: 1 point, usually: 2 points, a lot: 3 points, always: 4 points). The response data may be a selection signal for one of the plurality of selection items, and the control unit 130 may calculate (derive or compute) a cognitive distortion score of the pain patient using a score matched to the selection item according to the received selection signal.

Meanwhile, the control unit 130 may calculate a cognitive distortion score of the pain patient from the survey response data using various methods. For example, the control unit 130 may calculate a cognitive distortion score of the pain patient using a sum or average of the survey response data corresponding to the plurality of survey data. In this case, the control unit 130 may allocate (assign) different weights for each survey response data to calculate the cognitive distortion score of the pain patient.

The control unit 130 may compare the calculated cognitive distortion score to the predefined reference score 460a.

When the cognitive distortion score of the pain patient is higher than the predefined reference score 460a (i.e., above the predefined score) as a result of the comparison, the control unit 130 may categorize (distinguish, specify, or determine) the degree of cognitive distortion of the pain patient as the “first type of degree of cognitive distortion (high cognitive distortion, 461)”. In contrast, when the cognitive distortion score of the pain patient is lower than the predefined reference score 460a (i.e., equal to or less than the predefined cognitive distortion score), the control unit 130 may categorize the degree of cognitive distortion of the pain patient as the “second type of degree of cognitive distortion (low cognitive distortion, 462)”.

That is, the control unit 130 may determine the degree of cognitive distortion of the pain patient as one of high cognitive distortion or low cognitive distortion, depending on whether the cognitive distortion score exceeds the reference score.

Meanwhile, in the present invention, a process of specifying a user group corresponding to the state information on the pain patient among a plurality of user groups categorized according to pain duration and degree of cognitive distortion may be performed (S230, see FIG. 8B).

As illustrated in FIG. 8D, in the present invention, a plurality of user groups 410, 420, 430, and 440 may be categorized based on the pain duration 450 and degree of cognitive distortion 460. For convenience of description, in the present invention, an example is provided in which there are “four” different user groups based on the pain duration 450 and degree of cognitive distortion 460. Each of the user groups in the present invention may be referred to as a first user group 410, a second user group 420, a third user group 430, and a fourth user group 440. However, in the present invention, it is obvious that condition items and the number of conditions to categorize the user group may be set variously.

Meanwhile, in each of the plurality of user groups 410, 420, 430, and 440, categorization reference information that categorizes the plurality of user groups may exist to be matched. This categorization reference information may be predefined and exist on the storage unit 120 or on an external server.

Specifically, in the first user group 410, a first type of pain duration (e.g., acute pain, 451) and a first type of degree of cognitive distortion (e.g., high cognitive distortion, 461) may exist to be matched as the categorization reference information.

In the second user group 420, the first type of pain duration (e.g., acute pain, 451) and a second type of degree of cognitive distortion (e.g., low cognitive distortion, 462) may exist to be matched as the categorization reference information.

In the third user group 430, a second type of pain duration (e.g., chronic pain, 452) and the first type of degree of cognitive distortion (e.g., high cognitive distortion, 461) may exist to be matched as the categorization reference information.

Further, in the fourth user group 440, the second type of pain duration (e.g., chronic pain, 452) and the second type of degree of cognitive distortion (e.g., low cognitive distortion, 462) may exist to be matched as the categorization reference information.

The control unit 130 may determine, among the plurality of user groups 410, 420, 430, and 440, a specific user group in which the categorization reference information corresponding to the state information of the pain patient is matched.

Specifically, when the state information of a first pain patient (e.g., “patient Cheolsoo Kim”) includes the first type of pain duration (e.g., acute pain, 451) and the first type of degree of cognitive distortion (e.g., high cognitive distortion, 461), the control unit 130 may specify (or categorize) the first pain patient as a pain patient belonging to the first user group 410 among the plurality of user groups.

Further, when the state information detected from the survey response data of a second pain patient (e.g., “patient Youngsuk Lee”) includes the first type of pain duration (e.g., acute pain, 451) and the second type of degree of cognitive distortion (e.g., low cognitive distortion, 462), the control unit 130 may specify (or categorize) the second pain patient as a pain patient belonging to the second user group 420 among the plurality of user groups.

Further, when the state information of a third pain patient (e.g., “patient Mincheol Choi”) includes the second type of pain duration (e.g., chronic pain, 452) and the first type of degree of cognitive distortion (e.g., high cognitive distortion, 461), the control unit 130 may specify (or categorize) the third pain patient as a pain patient belonging to the third user group 430 among the plurality of user groups.

Further, when the state information of a fourth pain patient (e.g., “patient Minji Bang”) includes the second type of pain duration (e.g., chronic pain, 452) and the second type of degree of cognitive distortion (e.g., low cognitive distortion, 462), the control unit 130 may specify (or categorize) the fourth pain patient as a pain patient belonging to the fourth user group 440 among the plurality of user groups.

Meanwhile, in the present invention, a process of determining an initial therapy protocol corresponding to the user group among the plurality of therapy protocols may be performed (S240, see FIG. 8B).

As illustrated in FIGS. 8E and 8F, the therapy protocol according to the present disclosure may be configured with a plurality of therapy programs 510 to 580, each matched with different topics 510a to 580a. That is, each therapy program means a program for performing therapy according to a topic matched to each therapy program.

Here, “topics 510a to 580a” correspond to the plurality of programs 510 to 580 for cognitive behavioral therapy of the pain patient, respectively. For example, as illustrated in FIGS. 8G and 8H, i) motivation enhancement (hereinafter, a first topic 510a) may correspond to a first program 510, ii) emotion identification (hereinafter, a second topic 520a) may correspond to a second program 520, iii) behavioral strategy (hereinafter, a third topic 530a) may correspond to a third program 530, iv) attention shift (hereinafter, a fourth topic 540a) may correspond to a fourth program 540, v) thought shift (hereinafter, a fifth topic 550a) may correspond to a fifth program 550, vi) thought record (hereinafter, a sixth topic 560a) may correspond to a sixth program 560, vii) management strategy (hereinafter, a seventh topic 570a) may correspond to a seventh program 570, and viii) future me (hereinafter, an eighth topic 580a) may correspond to an eighth program 580.

The first topic (motivation enhancement) is directed to understanding cognitive behavioral therapy and schema for pain and increasing therapy motivation by establishing the pain patient's own goals, and may be configured with therapy modules related thereto.

The second topic (emotion identification) is directed to identifying pain, emotions, and the pain patient's own coping styles, and may be configured with therapy modules to identify the pain patient's negative emotions related to pain, physical reactions, and behaviors, and to identify the pain patient's own coping styles.

The third topic (behavioral strategy) is therapy for pain patients to set new coping behavior ways by setting activity goals and to cope with pain using breathing and progressive relaxation techniques, which may be configured with therapy modules therefor.

The fourth topic (attention shift) is a therapy for coping with pain using an attention shifting method that uses activities, emotions, and the five senses, and may be configured with therapy modules therefor.

The fifth topic (thought shift) is directed to exploring negative automatic thoughts related to pain, identifying feelings and behaviors based on automatic thoughts, and engaging in a process of finding evidence to counter the automatic thoughts, which may be configured with therapy modules therefor.

The sixth topic (thought record) is a process of exploring the pain patient's own irrational thought patterns and finding cognitive flexibility, which may be configured with therapy modules therefor.

The seventh topic (management strategy) is directed to performing positive self-talk and practicing pain management strategy, which may be configured with therapy modules to use positive self-talk to cope with pain and to understand the “stop-think-evaluate-act” method.

The eighth topic (future me) is related to overcoming obstacles (treatment resistance) and developing a positive self-image, and may be configured with therapy modules to organize previously learned coping methods, create the pain patient's own recipe for coping with pain (pain coping recipe), and identify alternatives to expected difficulties.

Meanwhile, in the present invention, topics 510a to 580a may be predefined and exist in the storage unit 120. Meanwhile, it is obvious that the number and content (or kinds) of topics in the present invention are not limited to the examples described above and may be defined in various ways.

The specific therapy program may be configured to include at least one therapy module related to a specific topic matched to the specific therapy program. For example, the first therapy program 510 may be configured with a plurality of therapy modules 511 to 515 related to the first topic (e.g., “motivation enhancement”, 510).

Here, the term “therapy module” may mean contents related to a detailed category (or subtopic) for cognitive behavioral therapy of the pain patient for a specific topic. For example, the plurality of therapy modules 511 to 515 related to the first topic (e.g., “motivation enhancement”, 510a) may include “what pain means to me therapy module 511”, “pain schema therapy module 512”, “pain questionnaire therapy module 513”, “set goals therapy module 514”, and “practice recording pain therapy module 515” (see FIG. 8E). In the present invention, a therapy module included in a therapy program related to a specific topic may be referred to as a “therapy module corresponding to specific topic”. Further, in the present invention, the term “therapy module” may be used interchangeably with the term “chapter”.

Meanwhile, as illustrated in FIG. 8D, there may be different therapy protocols 410a, 420a, 430a, and 440a matched to each of the plurality of user groups 410, 420, 430, and 440.

The plurality of therapy protocols 410a, 420a, 430a, and 440a matched to each of the plurality of user groups 410, 420, 430, and 440 may be configured with at least one different therapy program or therapy module, depending on the characteristics of the pain duration 450 and degree of cognitive distortion 460 of the user group 410, 420, 430, and 440.

For example, the first therapy protocol 410a matched to the first user group 410 and the third therapy protocol 430a matched to the third user group 430 may be configured to include all therapy modules (full modules) stored on the server. In this case, all therapy modules include all of the plurality of therapy modules matched to each of the first to eighth topics, and cognitive behavioral therapy may be performed using all of the therapy modules included in each of the first to eighth topics for a pain patient belonging to the first user group.

In another example, a therapy protocol matched to the second user group 420 and the fourth user group 440 may be configured to include some of the therapy modules, but not all of the therapy modules (full modules) stored on the server. The therapy protocol matched to the second user group 420 and the fourth user group 440 may include only some of the therapy modules of the plurality of therapy modules matched to each of the first to eighth topics, and cognitive behavioral therapy may be performed for the pain patient belonging to the second user group 420 and the fourth user group 440 using some of the therapy modules among the therapy modules constituting the first to eighth topics.

In one example, the second therapy protocol 420a matched to the second user group 420 may be configured to focus on modules related to coping methods, breathing techniques, and disease education. In another example, the fourth therapy protocol 440a matched to the fourth user group 440 may be configured to focus on modules related to acceptance commitment.

Meanwhile, as illustrated in FIGS. 8E and 8F, each of the plurality of therapy programs 510 to 580 may include worksheet modules 515, 522, 531, 541, 551, 561, 571, and 581 for reviewing at least one of a degree of pain, pain duration, a mental health state, or a physical health state of the patient.

In this case, user response information may be input into each of the worksheet modules for reviewing at least one of the degree of pain, pain duration, mental health state, and physical health state of the pain patient in relation to a specific topic of the therapy program in which the worksheet module is included. Further, various user response information may be input into each of the worksheet modules for reviewing at least one of the degree of pain, pain duration, mental health state, and physical health state of the pain patient, as well as contents related to a specific topic of the therapy program.

For example, the user response information may be input into the worksheet module 515 included in the first therapy program 510 to review at least one of the degree of pain, pain duration, mental health state, and physical health state of the pain patient in relation to the first topic (“motivation enhancement”, 510a).

In another example, the user response information may be input into the worksheet module 522 included in the second therapy program 520 to review at least one of the degree of pain, pain duration, mental health state, and physical health state of the pain patient in relation to the second topic (“emotion identification”, 520a).

The worksheet module may be provided at various occasions in each therapy program. For example, the worksheet module may be positioned at the very last or in the middle of each therapy module. The position where the worksheet module is included in each therapy program may vary depending on the state of the pain patient.

Meanwhile, the worksheet module may be provided as the very last module in each therapy program. The worksheet module may be disposed as the very last module among the therapy modules constituting each therapy program, so that the pain patient proceeds to the worksheet module after completing the therapy modules of the corresponding therapy program. This may include the purpose of receiving a therapy effectiveness of the pain patient, objectification, or feedback from the pain patient on the therapy program.

Further, the worksheet modules included in each of the therapy programs may be configured to allow the pain patient to score (select a score, input a score, etc.) the severity of pain, mood when experiencing pain, degree of negative emotions for pain, degree of stress for pain, and the like. The control unit 130 may judge, using the score received from the worksheet module, whether the therapy program including the worksheet module was helpful and effective for the pain patient.

For example, when there is a therapy program with a low pain score, the control unit 130 may judge that the corresponding therapy program was helpful to the user. In this case, the corresponding therapy program may be reflected in an update of the therapy protocol described below.

Meanwhile, in the present invention, a process of sequentially providing a plurality of specific therapy programs included in the initial therapy protocol to the user terminal 10 according to a therapy week set for each of the plurality of specific therapy programs may be performed (S250, see FIG. 8B).

Here, the term “therapy week” may be understood as a sequence (or period) in which the therapy program is provided (or activated) through the user terminal 10 for the patient to perform cognitive behavioral therapy according to the therapy program. In the present invention, the “total number of times” of therapy weeks and the “therapy period for each week” corresponding to each therapy week may be preset and exist. Further, in the present invention, it may be understood that the “overall therapy period” is predefined. The overall therapy period may be determined as the product of the preset total number of times and the preset therapy period for each week. For example, when the total number of times is “eight” and the therapy period for each week is one week (seven days), the preset overall therapy period may be “eight weeks”.

Hereinafter, for convenience of description, an example in which a different therapy week arrives for each one-week therapy period “eight times” will be described. That is, in the present invention, a first therapy week may arrive in a first therapy session, and a second therapy week may arrive in a second therapy session. Accordingly, in the present invention, the term “therapy week” may be used interchangeably with “therapy session”, “therapy round”, “therapy period”, and “therapy sequence”.

The control unit 130 may set at least one therapy week for each of the plurality of therapy programs included in the initial therapy protocol. Further, the control unit 130 may, in a specific therapy week, provide a therapy program for which the specific therapy week is set.

As described above, in the present invention, a total number of times of a therapy week (e.g., “8”) may be predefined and exist.

The control unit 130 may set a different therapy week for each of the plurality of therapy programs when the plurality of therapy programs included in the initial therapy protocol correspond to the predefined number of times of the therapy week. For example, when a first therapy program to an eighth therapy program is selected in the initial therapy protocol, the control unit 130 may set one of the first therapy week to the eighth therapy week for each of the first therapy program to the eighth therapy program.

Meanwhile, the first topic to the eighth topic as described above may correspond to the first therapy program to the eighth therapy program, respectively. That is, i) the first therapy program may be for training or therapy related to the first topic (motivation enhancement), ii) the second therapy program may be for training or therapy related to the second topic (emotion identification), iii) the third therapy program may be for training or therapy related to the third topic (behavioral strategy), iv) the fourth therapy program may be for training or therapy related to the fourth topic (attention shift), v) the fifth therapy program may be for training or therapy related to the fifth topic (thought shift), vi) the sixth therapy program may be for training or therapy related to the sixth topic (thought record), vii) the seventh therapy program may be for training or therapy related to the seventh topic (management strategy), and viii) the eighth therapy program may be for training or therapy related to the eighth topic (future me).

Meanwhile, the control unit 130 may set a plurality of therapy weeks for at least some of the plurality of therapy programs when the plurality of therapy programs included in the initial therapy protocol falls short of the predefined number of times of the therapy weeks. The control unit 130 may repeatedly allocate the same topic to different weeks, such that the therapy is performed according to the predefined therapy week. For example, when the predefined therapy week is 8 weeks and there are seven therapy programs selected in the initial therapy protocol, the control unit may repeatedly allocate one of the seven therapy programs to a specific week. For example, the control unit 130 may repeatedly set the same topic (e.g., thought record, 560a) in the sixth week and the seventh week.

Meanwhile, the control unit 130 may, based on the initial therapy protocol, sequentially provide the plurality of specific therapy programs to the user terminal 10 according to the therapy week set for each of the plurality of specific therapy programs.

The control unit 130 may, based on the initial therapy protocol, activate a state of the specific therapy program such that, during a therapy period corresponding to the specific therapy week, the specific therapy program for which the specific therapy week is set may be provided on the user terminal 10. Hereinafter, to avoid terminological confusion, a state of a specific therapy program will be described to be referred to as a “mode” of a specific therapy program.

In the present invention, a “therapy program activation mode” may be understood as a mode in which at least some of the plurality of therapy modules included in the therapy program are available for being viewed (or used). In contrast, a “therapy program deactivation mode” may be understood as a mode in which all of the plurality of therapy modules included in the therapy program are not available for being viewed (or used).

For example, as illustrated in FIG. 8E, the control unit 130 may, based on the arrival of the first therapy week (first week), activate a mode of the first therapy program 510 in which the first therapy week is set. Further, the control unit 130 may, based on the arrival of the second therapy week (second week), activate a mode of the second therapy program 520 in which the second therapy week is set.

Accordingly, the patient may be systematically provided with cognitive behavioral therapy using the plurality of therapy programs sequentially according to the therapy week set in the initial therapy protocol.

Meanwhile, the control unit 130 may sequentially provide specific therapy modules constituting the plurality of specific therapy programs to the user terminal 10, according to the therapy week set for each of the plurality of specific therapy programs.

The control unit 130 may sequentially provide the plurality of therapy modules 511 to 515 constituting the first therapy program 510 to the user terminal 10 in the first therapy week (see FIG. 8E).

Here, “therapy module sequence” may be understood as a sequence in which the plurality of therapy modules included in a specific therapy program are provided.

Further, the “therapy module activation mode” may be understood as a mode in which the therapy module is available for being viewed (or used). In contrast, the “therapy module deactivation mode” may be understood as a mode in which the therapy module is not available for being viewed (or used).

The control unit 130 may, depending on the sequence of therapy modules, change (or switch) a mode of the therapy module corresponding to the next turn in the sequence from a deactivation mode to an activation mode based on the completion of the cognitive behavioral therapy according to the therapy module corresponding to a specific sequence.

For example, as illustrated in FIG. 8E, assume that the first therapy program 510 is configured with the first therapy module to the fifth therapy module 511 to 515. The control unit 130 may switch a mode of the second therapy module 512 from a deactivation mode to an activation mode based on the completion of the cognitive behavioral therapy of the first therapy module 511.

Meanwhile, the control unit 130 may switch a mode of the therapy module with the earliest sequence among the plurality of therapy modules constituting a specific therapy program from a deactivation mode to an activation mode based on a mode of the specific therapy program being activated.

For example, the control unit 130 may switch a mode of the first therapy module 511 corresponding to the first priority (first sequence) of the plurality of therapy modules 511 to 515 included in the first therapy program 510 from a deactivation mode to an activation mode based on the first therapy program 510 being activated.

In this case, the first priority therapy module in the second week therapy program to the eighth week therapy program 520 to 580, excluding the first week therapy program 510, may be the first therapy module in which the last worksheet module review module including the user response information for the worksheet module provided in the previous therapy week corresponds to the first priority in sequence.

The control unit 130 may, in order to make the pain patient aware of the past state of the pain patient, prioritize providing the user response information for the worksheet module provided in the therapy week prior to the current therapy week before providing the therapy program corresponding to the current therapy week among the plurality of specific therapy programs.

For example, the control unit 130 may prioritize providing the user response information for the worksheet module provided in the first therapy program to the user terminal 10 before providing the second therapy program. Further, the user response information for the worksheet module provided in the previous therapy week may be provided at various occasions.

Meanwhile, in the present invention, “specific therapy program is activated” may be understood as switching the mode of the last worksheet module corresponding to the first priority (e.g., first sequence) among the plurality of therapy modules constituting the specific therapy program from a deactivation mode to an activation mode.

Meanwhile, the control unit 130 may collect, from the user terminal 10, therapy response data from specific therapy modules.

The control unit 130 may, based on a specific therapy module being selected that is activated through the user terminal 10, provide a page corresponding to the specific therapy module to the user terminal 10.

The page corresponding to the specific therapy module may include cognitive behavioral therapy content related to a specific topic. For example, a page corresponding to the therapy module 511 included in the first therapy program 510 may include cognitive behavioral therapy content (e.g., “What pain means to me?”) related to the first topic (e.g., “motivation enhancement”, 510a). Hereinafter, for convenience of description, a page corresponding to a specific therapy module will be referred to as a “specific topic related page”.

The control unit 130 may, through a specific topic related page provided to the user terminal 10, collect therapy response data according to the cognitive behavioral performance of the pain patient related to a specific therapy module.

Then, the control unit 130 may match the therapy response data collected through the page corresponding to the specific therapy module with the specific topic and store the therapy response data in the storage unit 120. In this case, the specific topic matched with the therapy response data may mean a topic related to the specific therapy module.

For example, assume that a page corresponding to a specific therapy module (e.g., a pain recording practice page) is provided on the user terminal 10 based on a specific module (one of 511 to 515) included in the first program 510 being selected. The control unit 130 may match therapy response data input through the page above with the first topic (e.g., “motivation enhancement”, 510a) and store the therapy response data in the storage unit 120 (see FIG. 8E).

In another example, assume that a page corresponding to a specific module is provided on the user terminal 10 based on a specific module 521 or 522 included in the second program 510 being selected. The control unit 130 may match therapy response data input through the page above with the second topic (e.g., “emotion identification”, 520a) and store the therapy response data in the storage unit 120 (see FIG. 8E).

Meanwhile, the control unit 130 may configure a page such that the pain patient inputs therapy response data required by a specific therapy module on the page corresponding to a specific therapy module.

For example, the control unit 130 may, in order to collect therapy response data related to a perception of pain intensity of the pain patient, display question data questioning the pain intensity (e.g., “How is your pain today?”) in one area of the page, and graphic objects corresponding to each of a plurality of pain intensities (e.g., “Pain intensity 1 to pain intensity 5”) in a different area of the page. Further, the control unit 130 may collect therapy response data for the pain intensity of the patient based on a specific graphic object being selected on the page.

In another example, the control unit 130 may, in order to collect therapy response data for an emotion shift method of the pain patient, provide a text input field on one area of the page in which the pain patient may input an emotion shift method that the pain patient desires to attempt. The control unit 130 may collect therapy response data related to the emotion shift method that is input into the text input field.

Meanwhile, the control unit 130 may provide a page including various cognitive behavioral therapy contents on the user terminal 10 for the cognitive behavioral therapy of the pain patient, in addition to the examples described above. Further, the control unit 130 may collect various therapy response data required for the cognitive behavioral therapy through the page.

Meanwhile, in the present invention, based on a result of the cognitive behavioral therapy of the pain patient proceeding on the basis of the initial therapy protocol (in particular, initial therapy response data), a therapy program (or a therapy module constituting a therapy program) corresponding after a specific therapy week can be changed.

Accordingly, an “initial therapy protocol” as described in the present invention may mean a therapy protocol determined based on survey response data, and an “update therapy protocol” may be understood as a protocol in which at least one of an initial therapy program or an initial therapy module included in the initial therapy protocol is changed based on the initial therapy response data.

Meanwhile, as information used to update the initial therapy protocol, various information may be used in addition to information collected from the therapy modules. For example, in the present invention, after therapy according to the initial therapy protocol is initiated, an investigation (information collection) of at least one of the patient's i) degree of pain, ii) degree of negative emotion (depression), iii) presence of insomnia, iv) degree of cognitive distortion (catastrophizing, risk avoidance), v) degree of stress due to pain, vi) most discomfort issue in daily life (occupation, interpersonal relationships, etc.), and vii) competence in coping with pain may be performed through a questionnaire once daily during a preset initial therapy period (e.g., initial 4 weeks).

For example, in the present invention, after the therapy according to the initial therapy protocol is initiated, an investigation (information collection) of viii) a therapy module or method that the patient subjectively finds most helpful in coping with pain may be performed at weekly intervals during the preset initial therapy period. The control unit 130 may update the initial therapy protocol based on an analysis of the information investigated above.

Further, in order to collect the information above, the control unit 130 may provide the user with an information collection alarm in various ways (e.g., an application push message, etc.), and when an answer for collecting the information above is not made within a preset time, an additional alarm may be provided. In this case, the time required to collect the information may be limited, and the control unit 130 may impose a preset evaluation time and ensure that, within the corresponding evaluation time, an evaluation of at least one of the items i) to viii) above is made.

The user's information input for items i) to viii) above may be used as “therapy response data” as described in the present invention. Meanwhile, it is obvious that the frequency and interval at which the therapy response data is collected may be modified in various ways.

Hereinafter, a method of updating the therapy protocol will be described in detail.

In the following, for convenience of description, an example with an overall therapy period of “8 weeks” will be described. However, this is an example for illustrative purposes only, and the therapy period may be variously set and changed by the patient and system administrator.

Meanwhile, the control unit 130 collects therapy response data related to the cognitive behavioral therapy according to the initial therapy protocol, and may, based on a preset initial therapy period (e.g., “4 weeks”) being elapsed out of the preset overall therapy period (e.g., “8 weeks”), update the initial therapy protocol using the collected initial therapy response data.

The control unit 130 may, in order to update the initial therapy protocol, analyze the state of the pain patient by at least one analysis category based on the initial therapy response data.

In this specification, the term “analysis category” refers to a category subject to analysis of therapy response data for the cognitive behavioral therapy of the pain patient, which may include, for example, at least one of emotion, pain, insomnia, cognitive distortion, stress, competence, or discomfort.

The control unit 130 may obtain (or derive, calculate, or compute), using the initial therapy response data, a state analysis result for at least one of the pain patient's i) degree of pain (related to a pain analysis category), ii) degree of negative emotion (related to an emotion analysis category), iii) degree of insomnia (related to an insomnia category), iv) degree of stress for pain (related to a stress analysis category), v) degree of competence in coping with pain (related to a competence category), vi) degree of discomfort in daily life, or vii) degree of cognitive distortion.

The control unit 130 may derive the state analysis result of the pain patient for each analysis category in various ways.

For example, the control unit 130 may, using an artificial intelligence model that has performed machine learning for analyzing the state of the pain patient, obtain the state analysis result of the pain patient for each analysis category. In this case, the state analysis result obtained using the artificial intelligence model may include information that specifies a specific analysis category (e.g., “insomnia”) in which a problematic symptom of the pain patient satisfies a preset reference among a plurality of analysis categories. Alternatively, the state analysis result obtained using the artificial intelligence model may include an analysis score for each of the plurality of analysis categories.

In another example, the control unit 130 may compare whether the analysis score for each of the plurality of analysis categories satisfies the preset reference to specify an analysis category for which the problematic symptom of the pain patient among the plurality of analysis categories satisfies the preset reference. The control unit 130 may calculate (or compute) the analysis score for each analysis category based on the therapy response data. For example, the control unit 130 may the analysis score using (or calculating) initial therapy response data corresponding to multiple choice questions. In addition, the control unit 130 may perform an artificial intelligence analysis of the initial response data made up of a text to calculate the analysis score for the initial response data made up of a text. The control unit 130 may compare each of the calculated analysis scores for each category to a preset problem score for each category, and specify an analysis category in which the analysis score exceeds the preset problem score as a category in which the pain patient has a problematic symptom.

Meanwhile, the control unit 130 may, using the state analysis result of the pain patient based on the initial therapy response data, update the initial therapy program such that at least one of the therapy program and the therapy module related to the specified category is included in the therapy program.

As illustrated in FIG. 81, the control unit 130 may, based on the therapy response data according to the first therapy program to the fourth therapy program 510 to 540 set in each of the first therapy week to the fourth therapy week, change at least some of the programs 550 and 560 of the fifth therapy program to the eighth therapy program 550 to 580 set in each of the fifth therapy week to the eighth therapy week to new programs 550′ and 560′.

In this case, the control unit 130 may change remaining therapy programs allocated to a remaining therapy period (5 to 8 weeks) excluding the preset initial therapy period (e.g., 1 to 4 weeks), and at least one of therapy modules constituting the remaining therapy programs, to be associated with the specified analysis category (e.g., “insomnia”).

As illustrated in FIG. 8J, assume that an initial therapy protocol 810 exists. The initial therapy protocol 810 may be configured to include a first initial therapy program to an eighth initial therapy program 811 to 818 related to different topics, at each of the first week and the eighth week. Further, each of the initial therapy programs 811 to 818 may include a therapy module corresponding to each topic. For example, the fifth initial therapy program 815 may include a plurality of initial therapy modules 815a to 815f associated with a fifth topic, and the sixth initial therapy program 816 may include a plurality of initial therapy modules 816a to 816f associated with a sixth topic.

As illustrated in FIG. 8K, the control unit 130 may, based on the state analysis result of the pain patient for the initial therapy response data, exclude at least some (815d to 815f and 816d to 816f) of the therapy modules 815a to 815f and 816a to 816f included in the remaining therapy programs 815 to 816. The remaining therapy programs 815 to 816 of an updated therapy protocol 820 may include at least some therapy modules 815a to 815c and 816a to 816c that are not excluded. In this case, the non-excluded therapy modules 815a to 815c and 816a to 816c may be associated with the specified analysis category.

Further, the control unit 130 may, based on the state analysis result of the pain patient for the initial therapy response data, reset a specific therapy program set in the preset initial therapy period (or initial therapy week) to at least a portion of the remaining therapy period (or remaining therapy week). That is, the control unit 130 may update the initial therapy protocol by reallocating (or resetting or reassigning) the therapy program associated with a specific topic matched to the preset initial therapy period (or initial therapy week) to the remaining therapy period (or remaining therapy week).

For example, as illustrated in FIG. 8L, the control unit 130 may reset the third therapy program 813 set in the third week to the fifth week, and the fourth therapy program 814 set in the fourth week to the sixth week. An updated therapy protocol 830 may include the previously performed therapy programs 813 and 814 duplicated (or repeated) at different weeks. That is, in the present invention, the initial therapy program may be updated based on the initial therapy response data such that the previously performed therapy program is performed again.

The duplicately or repeatedly performed therapy program may be a module that is judged to be most helpful to the pain patient. This judgment may be achieved through an analysis of evaluation information on the therapy program received from the pain patient, changes in values for pain (e.g., severity of pain, mood when experiencing pain, degree of negative emotions for pain, value (score) for degree of stress for pain) received through the worksheet module, frequency of use of coping method for pain created in the specific therapy program, time of use, etc. This analysis may be performed using a variety of artificial intelligence algorithms.

In this case, the reallocated therapy programs 813 and 814 may be associated with a specific analysis category.

Further, the control unit 130 may update the initial therapy program 810 such that the therapy program configured with the therapy modules corresponding to each of the different topics is set in the remaining therapy period (remaining therapy week).

For example, as shown in FIG. 8M, an updated protocol 840 may include a therapy program 841 that includes a combination of therapy modules corresponding to a plurality of topics. The therapy program 841 may include therapy modules 813a to 813c associated with a third topic and therapy modules 814a to 814c associated with a fourth topic, and may be set in the fifth therapy week. That is, at least some programs 841 of the therapy programs set in the remaining therapy period (remaining therapy week) may include some of the therapy modules provided in the preset initial therapy period (or initial therapy week).

Further, although not illustrated, the control unit 130 may update the initial therapy program by newly adding modules different from the modules set in the initial protocol to the remaining therapy period (remaining therapy week).

For example, assume that the fifth therapy program 815 constituting the initial protocol 810 included six fifth topic therapy modules 815a to 815f (see FIG. 8J). The control unit 130 may update the initial therapy protocol 810 by adding a new therapy module of the fifth topic that is not included in the therapy modules 815a to 815f according to the previously included fifth topic to the fifth therapy program 815.

In this case, the control unit 130 may exclude at least some of the previously included fifth topic therapy modules 815a to 815f and add new therapy modules. In addition, the control unit 130 may include new therapy modules, while leaving the previously included fifth topic therapy modules 815a to 815f unchanged. That is, the control unit 130 may include entirely new therapy modules that are different from the previously included therapy modules, in which case the previously included therapy modules may be excluded, retained, or modified in various ways.

Meanwhile, the control unit 130 may, based on an update result of the initial therapy protocol, change at least some of the example sentences provided by the therapy modules constituting the remaining therapy programs to be associated with a specified category.

As described above, the control unit 130 may specify, among the plurality of analysis categories, a category in which the problematic symptom of the pain patient satisfies the preset reference.

In this case, the control unit 130 may specify categories as many as a preset number (e.g., two) of the plurality of analysis categories. For example, the control unit 130 may specify the “depression” and “analysis” categories that have the highest analysis scores among the categories of emotion, pain, insomnia, cognitive distortion, stress, competence, and discomfort.

There may be example sentence information in the storage unit 120 that corresponds to at least one category that satisfies the preset reference. The control unit 130 may match at least one of information on the category satisfying the preset reference and example sentence information included in the category satisfying the preset reference to a patient's account and store it as matching information. Further, in the matching information, in addition to the categories satisfying the preset reference, information on selected example sentences selected by the pain patient among the example sentences provided in the therapy modules already performed by the pain patient may be stored. Further, the example sentences stored as the matching information for the category satisfying the preset reference may also be configured with the selected example sentences selected by the pain patient.

The control unit 130 may control such that, based on the example sentence information existing in the storage unit 120, the therapy programs 550 to 580 matched for the remaining therapy weeks include the example sentences associated with the specified analysis category. The control unit 130 may change (replace) at least some of the preset example sentences for the therapy modules constituting the therapy program matched to the remaining therapy weeks with selected example sentences stored in the example sentence information corresponding to the user history.

For example, assume that the depression category is specified. The control unit 130 may include an example sentence of “Ms. Hyeryun Jang, do you think that ‘my depressed heart can never be good again’” in a specific therapy module constituting the fifth therapy program set in the fifth week.

In another example, assume that the insomnia category is specified. The control unit 130 may include an example sentence of “Ms. Hyeryun Zhang, do you think that if you have pain, you can never sleep deeply” in a specific therapy module constituting the sixth therapy program set in the sixth week. As such, the control unit 130 may objectify the user's perception of the corresponding example sentence by continuously exposing the example sentence selected by the pain patient in the past or the example sentence of a specific category to the pain patient.

Meanwhile, in the present invention, based on the result of the cognitive behavioral therapy of the pain patient proceeding on the basis of the initial therapy protocol (in particular, the initial therapy response data), a judgment on whether to maintain the initial therapy protocol as it is or whether to change the initial therapy protocol may be performed.

The control unit 130 may, based on the result of the cognitive behavioral therapy of the pain patient, perform a judgment on whether to maintain the preset overall therapy period according to the initial therapy protocol. Further, the control unit 130 may, based on a judgment result, perform an update to the overall therapy period. Here, the update to the overall therapy period may include extending or shortening the overall therapy period. The control unit 130 may perform a judgment on whether to reduce (or stop or shorten) the overall therapy period, whether to maintain the overall therapy period as it is, or whether to extend the therapy period to a period longer than the overall therapy period.

The control unit 130 may perform the judgment above based on various references, and may make a judgment on stopping, maintaining, or extending the therapy for the pain patient, for example, based on whether the result of the cognitive behavioral therapy for the pain patient satisfies the preset reference (e.g., the reference set in relation to stopping, maintaining, or extending the therapy, respectively).

In an example, the control unit 130 may quantify the result of the cognitive behavioral therapy for the pain patient as a score or the like, and may make a judgment on stopping, maintaining, or extending the therapy for the pain patient based on which of a plurality of intervals the score is included. For example, when a result score of the cognitive behavioral therapy for the pain patient is included in a first interval (stopping interval), the control unit 130 may stop (or reduce) the cognitive behavioral therapy for the pain patient.

For example, when a result of the cognitive behavioral therapy for the pain patient is positive, the result of the cognitive behavioral therapy may be included in the score of the first interval. In this case, the control unit 130 may reduce the overall therapy period, in which case the control unit 130 may determine the period that is reduced. The control unit 130 may update the initial therapy protocol such that only the preceding therapy is performed up to an occasion when monitoring has been performed, and no subsequent therapy is performed. In this case, the cognitive behavioral therapy for the pain patient may be stopped.

In another example, when a result score of the cognitive behavioral therapy for the pain patient is included in a second interval (maintaining interval), the control unit 130 may maintain the cognitive behavioral therapy for the pain patient. In this case, the therapy period set in the initial protocol (e.g., 8 weeks) may remain the same. When the result of the cognitive behavioral therapy for the pain patient is moderate, the result of the cognitive behavioral therapy may be included in the score of the second interval. In this case, the control unit 130 may maintain the overall therapy period. Meanwhile, even in this case, the update to the initial protocol may be made, and the update to the initial protocol may be made as described above in conjunction with FIGS. 8J to 8M, and thus the specific description will be replaced by the above description.

In still another example, when a result score of the cognitive behavioral therapy for the pain patient is included in a third interval (maintaining interval), the control unit 130 may extend the therapy period of the cognitive behavioral therapy for the pain patient. In this case, the therapy period set in the initial protocol (e.g., 8 weeks) may be extended (e.g., 12 weeks, etc.). When the result of the cognitive behavioral therapy for the pain patient is negative, the result of the cognitive behavioral therapy may be included in the score of the third interval. In this case, the control unit 130 may further extend the overall therapy period. The control unit 130 may determine the extended period. The control unit 130 may determine the extent to which the period is extended based on the state of the pain patient. As the state of the patient is worse, the extended period may be longer. The control unit 130 may, when the therapy period is extended, perform a determination on the therapy program to be performed during the extended therapy period, and update the initial therapy program such that the determined therapy program is further allocated to the extended therapy period. Of course, the update to the initial therapy protocol may further include an update to the already allocated therapy program, as well as a decision on programs for the extended therapy period.

Meanwhile, the control unit 130 may use feedback on the therapy program performed by the pain patient to determine which therapy program to be further allocated for the extended treatment period. Further, the control unit 130 may use the state information on the pain patient for each analysis category analyzed based on the initial therapy response data for the previously performed therapy program to allocate the therapy program for the extended treatment period. The control unit 130 may, based on the state information on the pain patient for each analysis category of at least one of emotion, pain, insomnia, cognitive distortion, stress, competence, and discomfort, determine the category in which more therapy is to be performed on the pain patient, and ensure that the therapy program for the corresponding category is further allocated to the extended treatment period. Meanwhile, a method of determining the therapy program to be allocated for the extended therapy period may vary widely. For example, the control unit 130 may receive a patient's intent for an extension of the therapy program of the patient. An occasion for receiving the patient's intent may vary, for example, the intent on whether the patient wishes to extend the therapy period may be received from the patient at an occasion when the preset therapy period is completed or at an occasion when the preset therapy program is initiated. The control unit 130 may provide an interface for receiving information from the patient, with a pop-up page or in various other ways. The control unit 130 may extend the therapy program when receiving the intent to extend the therapy period from the patient. In this case, the extended period may also be selected by the patient. Further, at least one of a topic or therapy module of the extended therapy program may be selected by the user. Therefore, the patient can receive more effective therapy by selecting a therapy program or therapy module that is beneficial to himself/herself. As described above, a method and system for providing cognitive behavioral therapy for a pain patient according to the present invention can receive, through a user terminal, a plurality of survey response data, and, based on the survey response data, detect state information of the pain patient related to pain duration and a degree of cognitive distortion of the pain patient. Further, based on the state information of the pain patient, a therapy protocol for personalized cognitive behavioral therapy can be provided to the pain patient. As a result, the method and system of cognitive behavioral therapy according to the present invention can provide a personalized cognitive behavioral therapy program for the pain patient in consideration of the pain duration and degree of cognitive distortion of the pain patient even if the pain patient has the same disease, rather than providing a uniform cognitive behavioral therapy based on the disease of the pain patient. Further, the pain patient can be provided with the personalized cognitive behavioral therapy that fits his/her state.

Further, the method and system for providing cognitive behavioral therapy for a pain patient according to the present invention may sequentially provide a plurality of specific therapy programs according to a therapy week. This allows the pain patient to be provided with cognitive behavioral therapy in a systematic way so that the pain patient can complete the cognitive behavioral therapy without dropping out.

Further, the method and system for providing cognitive behavioral therapy for a pain patient according to the present invention can update the therapy program to help treat the pain patient based on therapy data collected in the process of performing the cognitive behavioral therapy, so that the updated cognitive behavioral therapy can be provided in consideration of the pain patient's remission, rather than the initial therapy program being provided continuously.

Meanwhile, the present invention described above may be executed by one or more processes on a computer and implemented as a program that can be stored on a computer-readable medium (or recording medium).

Meanwhile, as illustrated in (a) of FIG. 9A, the patient may select a fourth menu item 340 on the initial screen page 300 to be provided with an AI functional evaluation service based on an artificial intelligence model for the patient's exercise motion.

As illustrated in (b) of FIG. 9A, the control unit 130 may, based on the fourth menu item 340 being selected, provide a functional evaluation page 900 on the user terminal 10 that includes a preset plurality of exercise items (e.g., “Arms out to side,” “Raise arms forward,” and “Seated knee bends and extensions”). The exercise items that are subject to the AI functional evaluation service may be set and changed by an administrator of the system 100.

Meanwhile, the control unit 130 may control a camera provided on the user terminal 10 to photograph an exercise image of the patient U in order to perform exercise motion evaluation of the patient for the selected exercise item when one of the plurality of preset exercise items is selected. Hereinafter, the exercise image of the patient will be described to be referred to as an “image targeted for functional evaluation analysis”.

As illustrated in (a) of FIG. 9B, the control unit 130 may output a guidance message (e.g., “Please stand inside the screen”) on the user terminal 10 in order to detect a subject U corresponding to the patient from the image targeted for functional evaluation analysis photographed through the camera, such that the entire body of the patient is included within a specific area of the image targeted for functional evaluation analysis (or a user terminal display).

The control unit 130 may, based on the subject U corresponding to the entire body of the patient being included within the specific area, detect the subject U from the image targeted for functional evaluation analysis using an object detection algorithm.

The control unit 130 may use a variety of object detection algorithms. For example, the exercise therapy application 100 may use an algorithm (weighted box fusion (WBF)) that ensembles a plurality of bounding boxes. However, it is obvious that the control unit 130 is not limited to the object detection algorithm described above, but may use various object detection algorithms that are capable of detecting an object corresponding to the subject U from the image targeted for functional evaluation analysis.

Further, the control unit 130 may, based on the subject U corresponding to the entire body of the patient being detected within the specific area, photograph the image targeted for functional evaluation analysis including the patient performing an exercise motion according to a preset exercise item through the camera.

Then, the control unit 130 may extract keypoints P1 and P2 corresponding to preset joint points from the image targeted for functional evaluation analysis in real time in conjunction with the image targeted for functional evaluation analysis being photographed on the user terminal 10.

In this case, the control unit 130 may extract the keypoints P1 and P2 corresponding to the preset joint points from the image, based on the artificial intelligence model (artificial intelligence posture estimation model) that has performed training on training data including position information on the joint points.

Further, the control unit 130 may provide the extracted keypoints P1 and P2 on the user terminal 10 in real time such that the patient may intuitively recognize the joint points being analyzed for the exercise motion.

Specifically, as illustrated in (b) and (c) of FIG. 9B, the control unit 130 may output the image targeted for functional evaluation analysis on the user terminal 10 in real time in conjunction with the image targeted for functional evaluation analysis being photographed on the user terminal 10. Further, the control unit 130 may provide a graphic object corresponding to the extracted keypoint P1 or P2 by overlapping or rendering the graphic object on an area of the subject U corresponding to the preset joint point.

Further, the control unit 130 may, when a position of the preset joint point changes as the patient performs an exercise motion, provide a keypoint graphic object overlapping an area of the subject U corresponding to the changed joint point. That is, the control unit 130 may allow the keypoint graphic object to overlap the area corresponding to the joint point in the image targeted for functional evaluation analysis such that the position of the joint point that changes in real time is reflected.

Meanwhile, the control unit 130 may perform an analysis of the exercise motion corresponding to the exercise item performed by the patient using at least one of the image targeted for functional evaluation analysis or the keypoints P1 and P2.

In this case, the control unit 130 may, based on at least one of the artificial intelligence model (artificial intelligence motion analysis model) that has performed machine learning for analyzing the exercise motion or a predefined rule (or rule information), perform an analysis of the exercise motion of the patient.

Meanwhile, the analysis of the exercise motion of the patient may be performed, in addition to the range of motion of a joint, for at least one of a distance of motion of a joint, a speed (or acceleration) of motion of a joint, a body balance of a subject (corresponding to a patient) included in an exercise image targeted for analysis, a body balance, or a body alignment state (e.g., an axial alignment state of a leg, a spinal alignment state, etc.).

As described above, in the present invention, it is possible to provide cognitive behavioral therapy in conjunction with rehabilitation exercises, rather than just providing the rehabilitation exercises to a patient, to monitor information on the patient's degree of pain, pain duration, and mental health state. Further, in the present invention, through the AI functional evaluation as described above, it is possible to perform an analysis of the exercise motion of the patient and extract information on a patient's ability to perform the exercise, a part of the body that lacks the ability to perform the exercise (e.g., a part of the body with weak muscles), etc. through the analysis.

Accordingly, in the present invention, it is possible to update the exercise plan of the patient using at least one of a cognitive behavioral therapy result or an exercise analysis result, rather than depending only on the evaluation information of the patient. In this case, the updating of the exercise plan of the patient may include various adjustments or changes related to the updating of the exercise plan described above, such as the adjustment of the difficulty of the exercise, the change of the type of exercise, etc.

For example, the control unit 130 may update the exercise plan, based on the cognitive behavioral evaluation result (or feedback, e.g., which may be received through the worksheet module or other modules) received from the patient in the process of cognitive behavioral therapy, and based on the patient's feedback on pain (example 1, negative feedback on pain (e.g., my legs can no longer function), and example 2, positive feedback on pain (e.g., I feel like I can run somehow)). For example, when negative feedback is received as the cognitive behavioral evaluation result, the control unit 130 may decrease the difficulty of the exercise in the exercise plan of the patient, or change the type of exercise. The change of type of exercise may be performed by changing the type of exercise performed before the feedback on the patient's pain was received to a different exercise. In another example, when positive feedback is received as the cognitive behavioral evaluation result, the control unit 130 may increase the difficulty of the exercise in the exercise plan of the patient, or change the type of exercise. The change of type of exercise may be performed by changing the exercise to more difficult exercise than the type of exercise performed before the feedback on the patient's pain was received.

In still another example, from the AI functional evaluation, the control unit 130 may perform an analysis of the exercise motion of the patient and extract information on a patient's ability to perform the exercise, a part of the body that lacks the ability to perform the exercise (e.g., a part of the body with weak muscles), etc. through the analysis. Further, the control unit 130 may, from the extracted information, update the exercise plan to strengthen a part in which the patient's exercise ability is weak, or update the exercise plan to avoid putting too much strain on the part in which the patient's exercise ability is weak. For example, when the patient's specific body part (e.g., left leg muscle) is weak as the functional evaluation result, the control unit 130 may analyze whether there is an exercise in the current exercise plan that puts too much strain on the patient's specific body part. The control unit 130 may, based on information on each type of exercise (e.g., description information), analyze whether there is an exercise that puts too much strain on the patient's specific body part. Further, when there is an exercise that puts too much strain on the patient's specific body part, the corresponding exercise may be excluded from the exercise plan. Further, the control unit 130 may extract an exercise item that may strengthen the patient's specific body part from the storage unit and include the exercise item in the exercise plan of the patient to update the exercise plan of the patient.

As described above, the control unit 130 may update the exercise plan of the patient based on at least one of state information on the patient according to the cognitive behavioral therapy or evaluation information according to the functional evaluation. Further, the control unit 130 may update the exercise plan of the patient based on at least one of the evaluation according to the state information and functional evaluation of the patient according to the cognitive behavioral therapy of the patient, or the evaluation information on the patient according to the evaluation page described above.

Meanwhile, as illustrated in FIG. 10, the control unit 130 may provide, on the user terminal 10, a summary page 1000 that includes results for the services provided by the present invention.

The control unit 130 may display, on the summary page 1000, at least one of i) rehabilitation exercise information according to the exercise plan, ii) evaluation information on the exercise plan, iii) AI functional evaluation information according to the AI functional evaluation, or iv) cognitive behavioral therapy result information according to the cognitive behavioral therapy plan.

As illustrated in FIG. 10A, the control unit 130 may display, on the summary page 1000, at least one of exercise performance rate information on each of the plurality of days constituting the rehabilitation period or average exercise performance rate information during the rehabilitation period as rehabilitation exercise information according to the exercise plan.

Further, as illustrated in FIG. 10B, the control unit 130 may display, on the summary page 1000, at least one of evaluation information (e.g., pain score, etc.) or information on a change in evaluation information (e.g., information comparing an initial pain score to a recent pain score) for each of the plurality of days constituting the rehabilitation period as the evaluation information on the exercise plan.

Further, as illustrated in FIG. 10C, the control unit 130 may display, on the summary page 1000, AI functional evaluation result information performed at a preset day interval during the rehabilitation period. In this case, the control unit 130 may display, on the summary page 1000, at least one of the number of performances (or performance time) or the average number of performances (or average performance time) of each of a plurality of preset exercise items.

Further, as illustrated in FIG. 10C, the control unit 130 may display, on the summary page 1000, AI functional evaluation result information performed at a preset day interval during the rehabilitation period. In this case, the control unit 130 may display, on the summary page 1000, at least one of the number of performances (or performance time) or the average number of performances (or average performance time) of each of a plurality of preset exercise items.

Further, as illustrated in FIG. 10D, the control unit 130 may provide, on the summary page 1000, a calendar that allows the user to identify whether the cognitive behavioral therapy is in progress according to the cognitive behavioral therapy plan. The calendar may display whether the worksheet module provided by the cognitive behavioral therapy plan has been performed. The control unit 130 may display graphic objects having different visual appearances on a day when the worksheet module is performed and on a day when the worksheet module is not performed.

As described above, the method and system for providing digitally-based musculoskeletal rehabilitation therapy according to the present invention can provide an exercise plan for musculoskeletal rehabilitation therapy to a patient through an application based prescription information including an exercise plan for the patient being allocated from the doctor terminal.

In particular, the method and system for providing digitally-based musculoskeletal rehabilitation therapy according to the present invention can play an exercise image corresponding to each of the plurality of exercise items, according to the plurality of exercise items constituting an exercise list, on the user terminal on which the application is executed. This allows a doctor to prescribe to a patient, and a patient to proceed rehabilitation through an exercise plan based on the doctor's prescription, even if the doctor and patient do not meet in person for rehabilitation therapy for a musculoskeletal disease, thereby resolving spatial, temporal, and economic constraints on the musculoskeletal rehabilitation therapy and increasing accessibility to the exercise therapy.

Further, the method and system for providing digitally-based musculoskeletal rehabilitation therapy according to the present invention can provide an evaluation page for performing an evaluation related to an exercise item based on a degree of playback of an exercise image satisfying a preset standard, and update an exercise plan based on evaluation information received through the evaluation page. This allows the patient to perform the exercise plan, provide appropriate feedback, and receive personalized rehabilitation therapy in which feedback is applied. In particular, the patient can be provided with individualized rehabilitation exercise therapy, such as adjusting the difficulty of exercise items according to the patient's state, and excluding exercise items that are difficult for the patient.

Further, the method and system for providing digitally-based musculoskeletal rehabilitation therapy according to the present invention can provide a cognitive behavioral therapy plan in conjunction with a rehabilitation exercise plan, thereby providing the patient with therapy for mental health as well as the rehabilitation site.

In particular, the method and system for providing digitally-based musculoskeletal rehabilitation therapy according to the present invention can receive, through the user terminal, survey response data for a plurality of survey data, and detect, based on the survey response data, state information on the patient related to pain duration and a degree of cognitive distortion of the patient. Further, based on the state information of the patient, a therapy protocol for personalized cognitive behavioral therapy can be provided to the patient. As a result, the method and system for providing rehabilitation therapy according to the present invention can provide the patient with a personalized cognitive behavioral therapy program for the patient in consideration of the pain duration and degree of cognitive distortion of the patient even if the patient has the same musculoskeletal disease, rather than providing the patient with a uniform cognitive behavioral therapy based on the musculoskeletal disease of the patient. Further, the patient can be provided with the personalized cognitive behavioral therapy that fits his/her state.

Further, the method and system for providing digitally-based musculoskeletal rehabilitation therapy according to the present invention can provide a plurality of therapy programs sequentially in conjunction with an exercise plan during a rehabilitation period. This allows the patient to complete rehabilitation therapy without dropping out, by systematically providing cognitive behavioral therapy along with the rehabilitation exercise.

Further, the method and system for providing digitally-based musculoskeletal rehabilitation therapy according to the present invention can update the therapy program to help the cognitive behavioral therapy of the patient based on therapy response data collected in the process of performing the cognitive behavioral therapy, so that the updated cognitive behavioral therapy can be provided in consideration of the patient's remission, rather than the initially determined method of cognitive behavioral therapy being provided continuously.

Meanwhile, as described above, the present invention may provide online-based exercise therapy for a musculoskeletal disease, and hereinafter, a method and system for providing exercise therapy will be described in more detail. In particular, hereinafter, a method and system for providing exercise therapy that is capable of analyzing an exercise motion of a patient performing a prescribed exercise based on an exercise image photographed of the exercise performed by the patient will be described. The present invention may provide a method and system for providing exercise therapy that is capable of analyzing an exercise motion of a patient from an exercise image based on an artificial intelligence model specialized for a musculoskeletal disease. Further, the present invention may provide a method and system for providing exercise therapy that is capable of providing a user environment in which a patient is easily accessible for a treatment of a musculoskeletal disease.

The present invention is directed to analyzing an exercise motion of a patient included in an exercise image based on the exercise image received from a patient terminal, and providing an analysis result. In particular, the present invention relates to a method of analyzing an exercise motion based on a joint point of a patient using an artificial intelligence model specialized for a musculoskeletal disease.

The present invention is described centered on an exercise motion analysis of a rehabilitation exercise for a musculoskeletal disease, but is not necessarily limited thereto. That is, in the present invention, a motion analysis may include not only an exercise motion, but also an analysis of a variety of motions, such as motions in daily life, motions during stretching, etc.

Meanwhile, the term “exercise motion” described in the present invention refers to a gesture (motion) made in the process of performing an exercise, and may be used interchangeably with the terms “motion”, “action”, “movement”, “gesture”, etc. of the body.

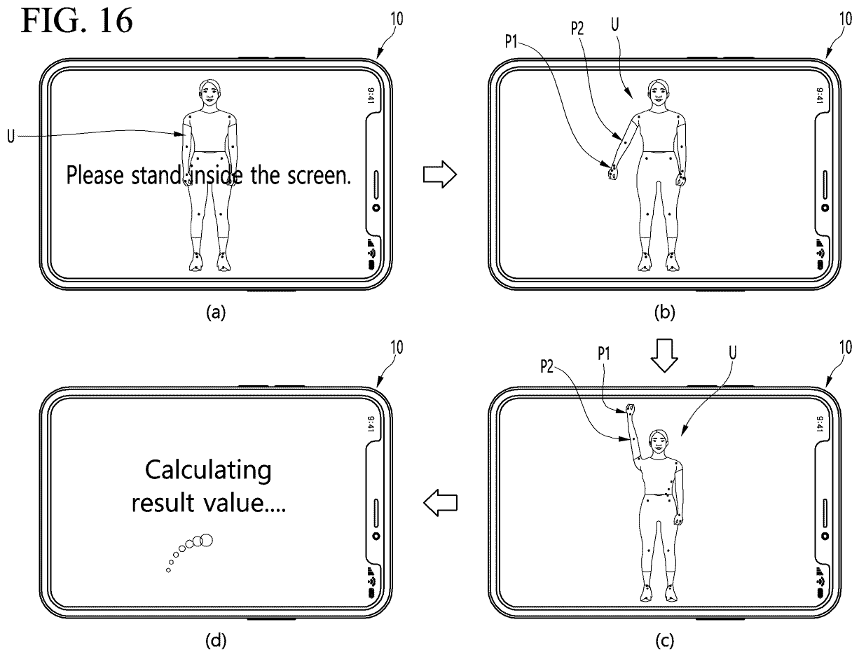

Further, the term “exercise image” refers to an image (or motion image) that photographs a process in which a patient performs an exercise motion, as illustrated in FIG. 16, which may include at least a portion of the body of the patient U.

In the present invention, a patient object included in an exercise image may be described to be referred to as “subject U”. In the present invention, the term “subject U” may mean a patient or a portion of the body of the patient who is exercising in the exercise image. In the present invention, the terms “subject” and “patient” may be used interchangeably and may be described by assigning the same reference numeral “U”.

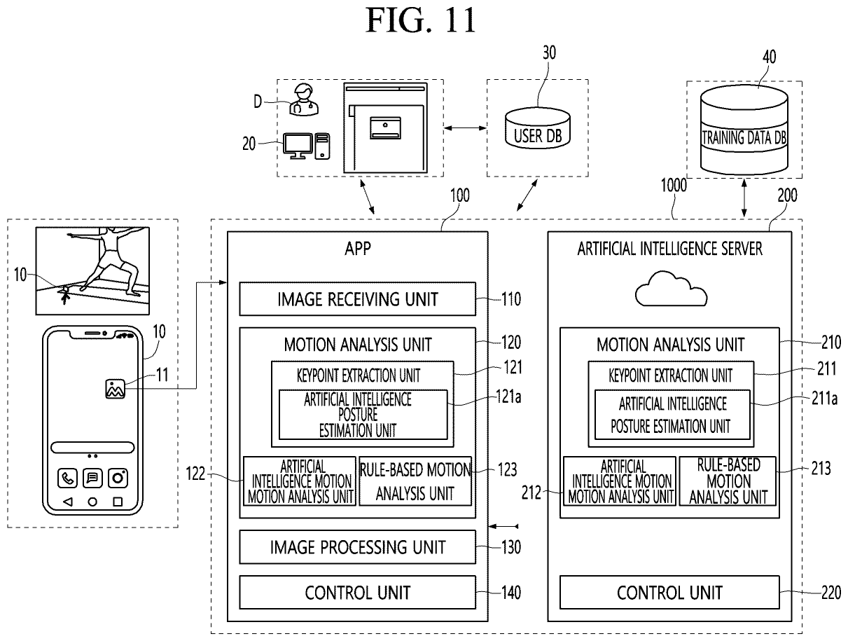

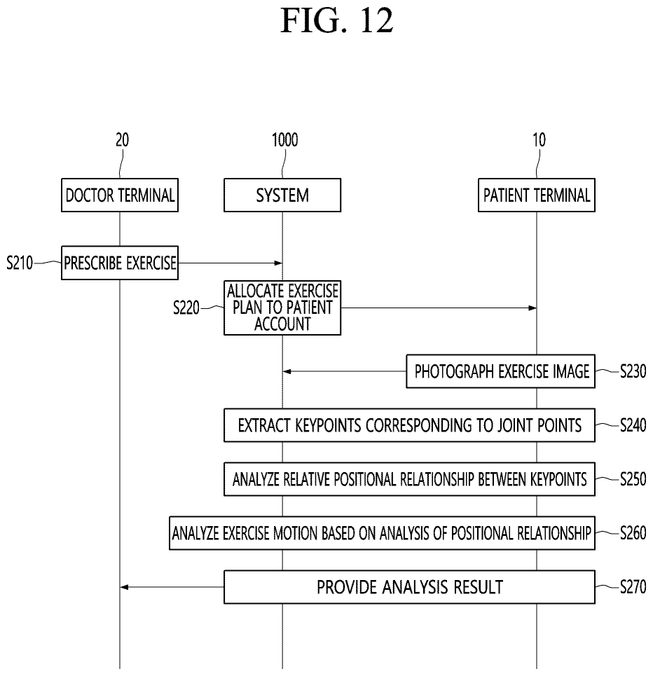

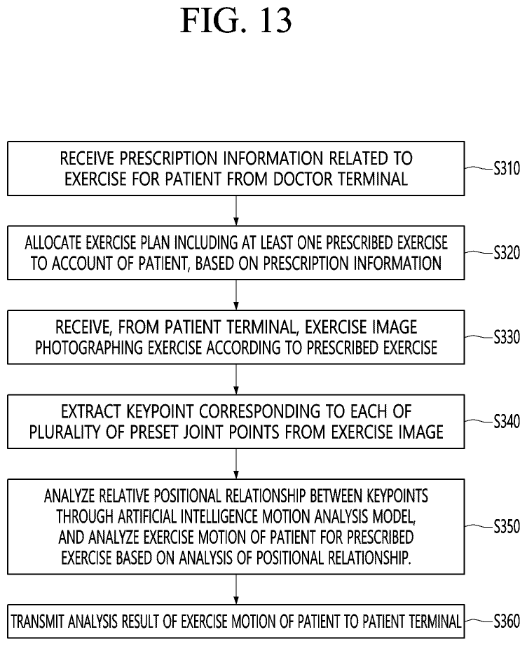

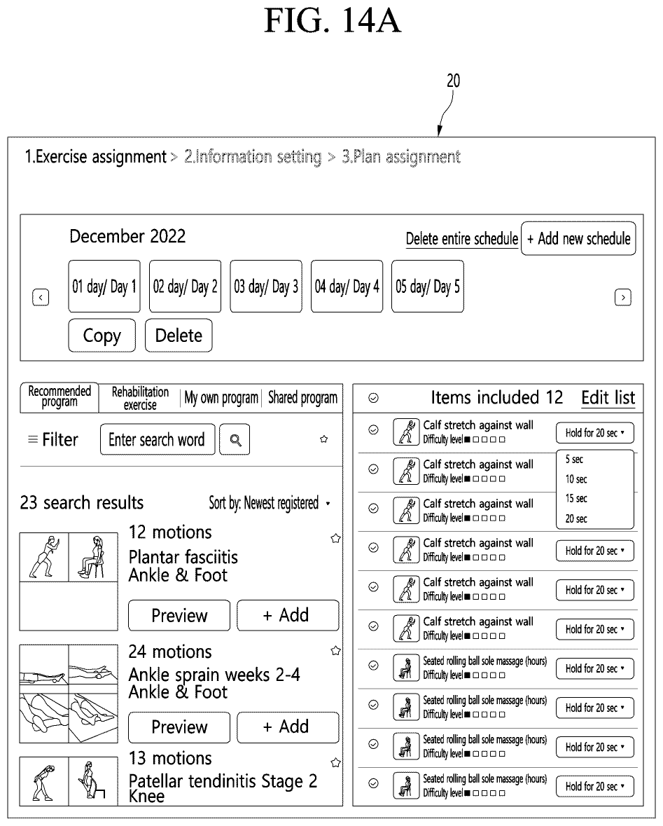

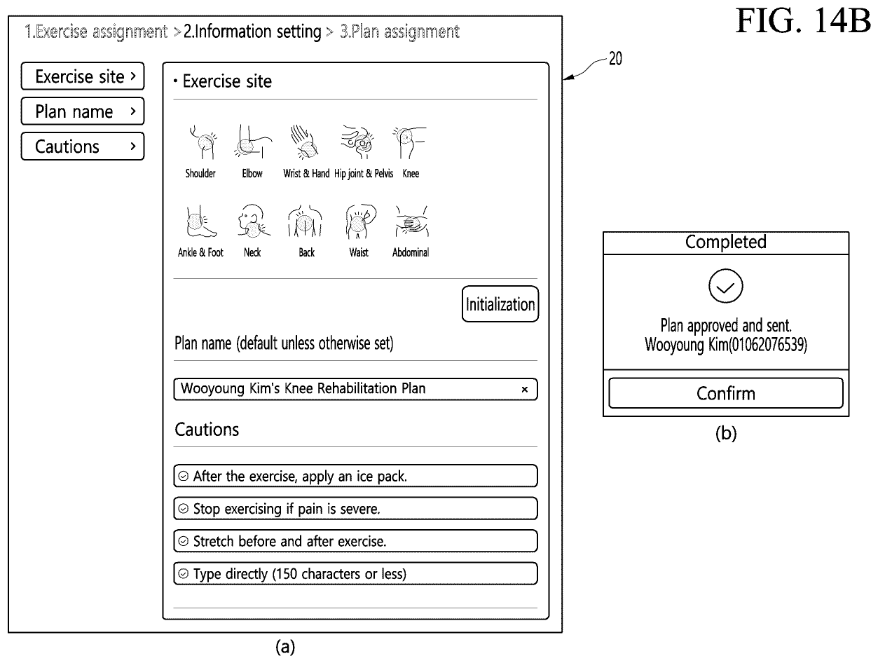

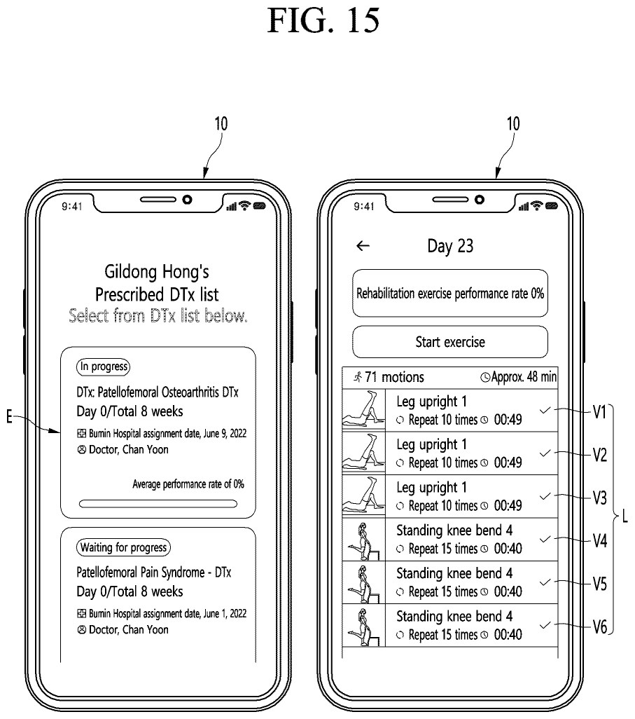

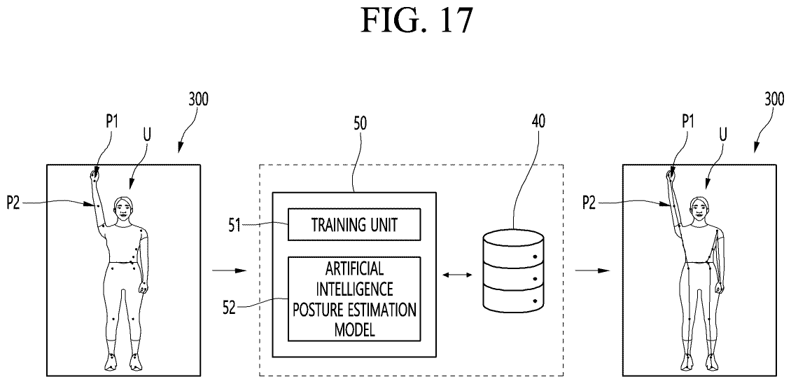

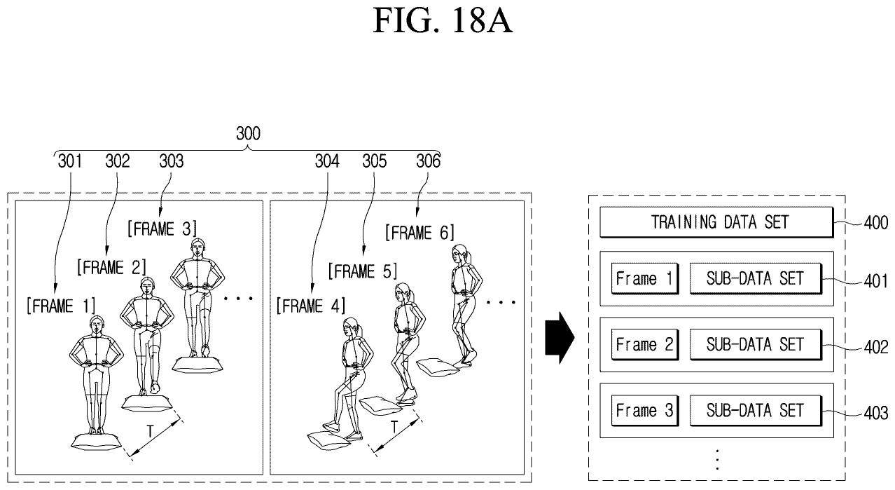

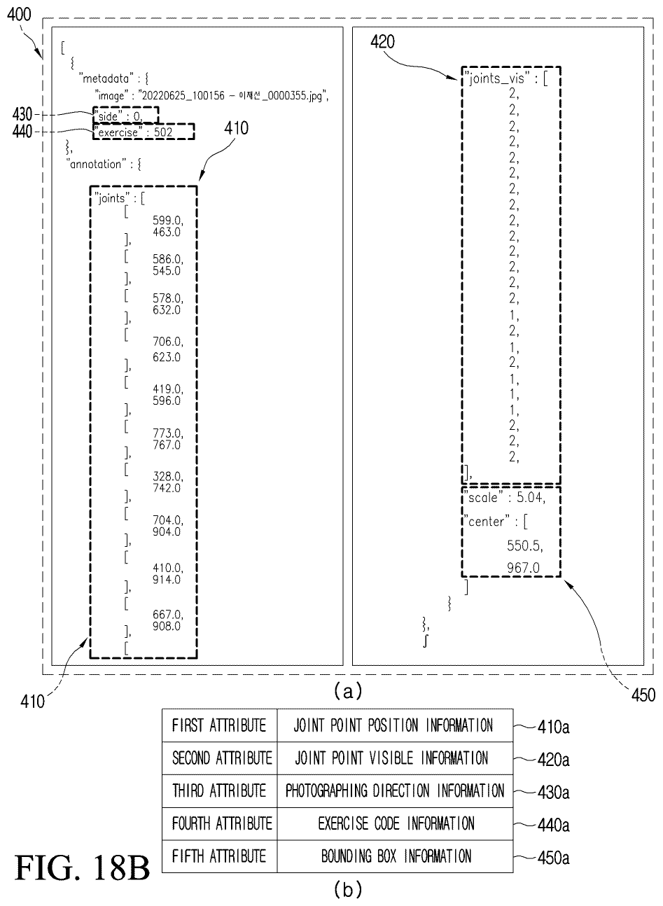

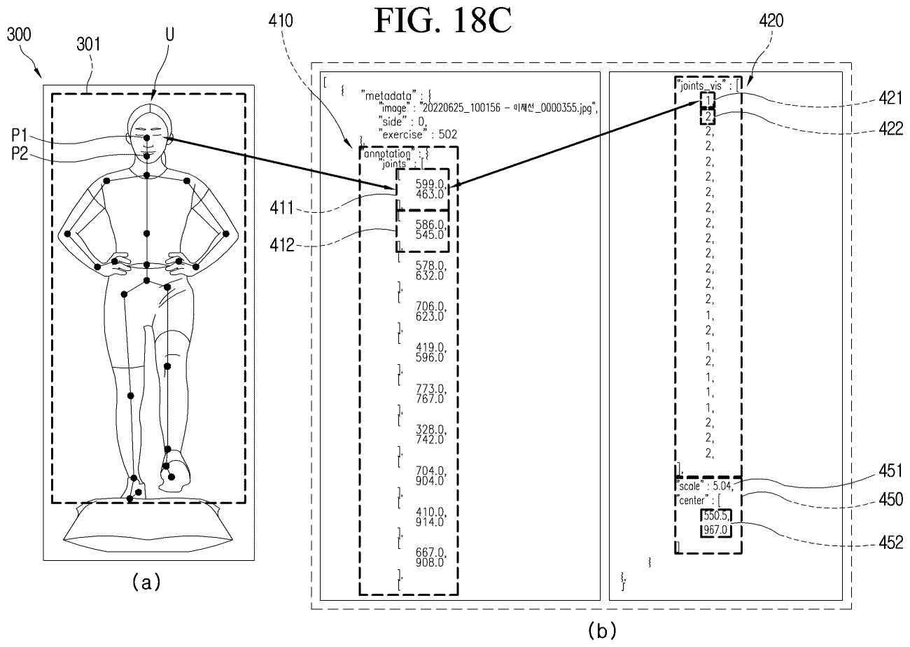

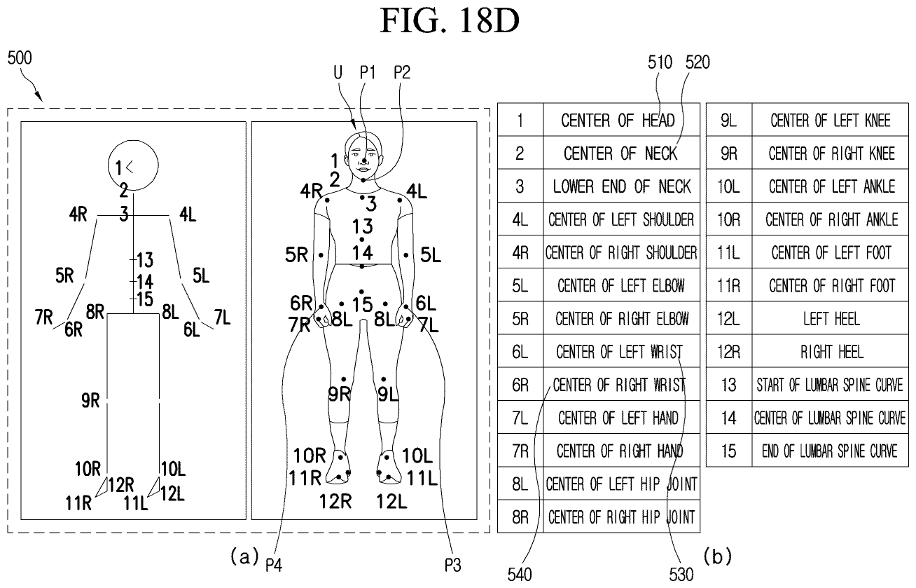

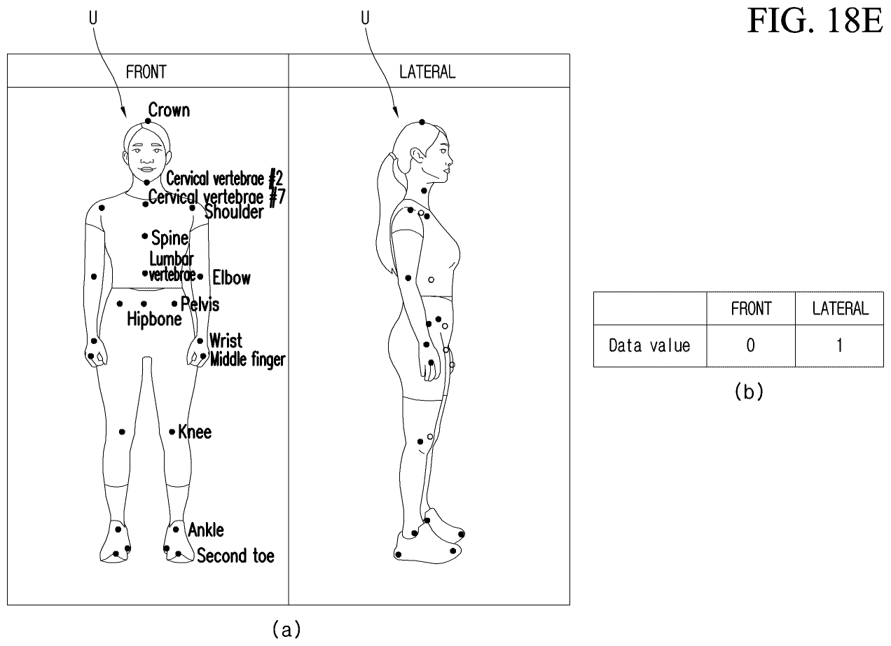

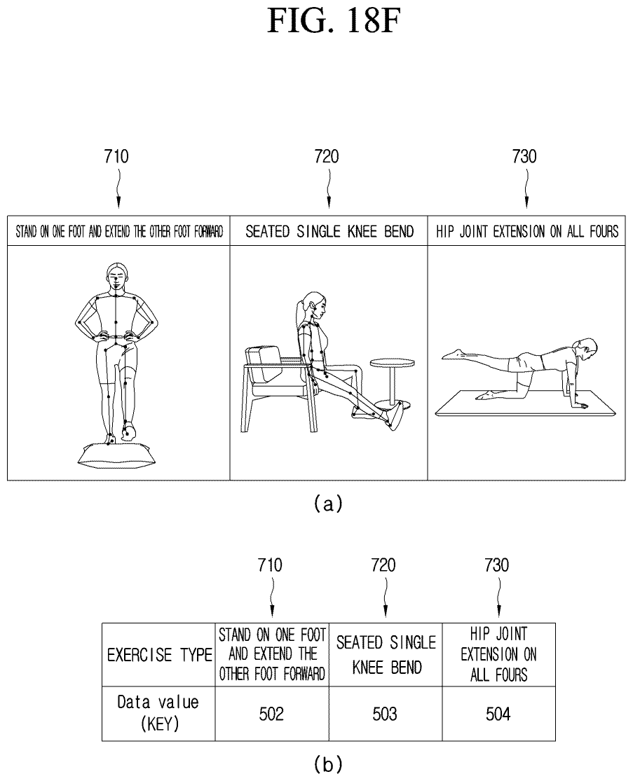

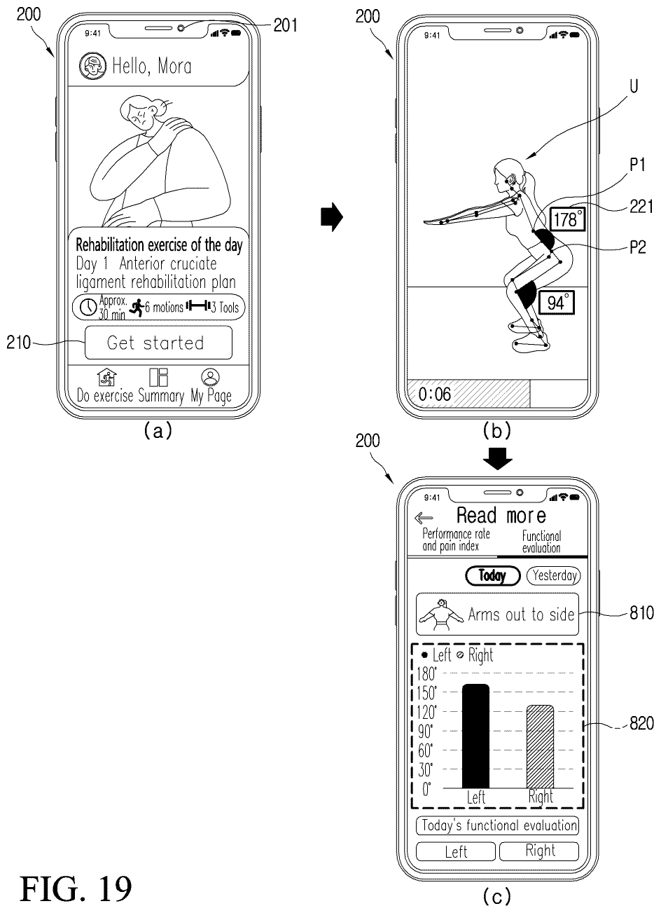

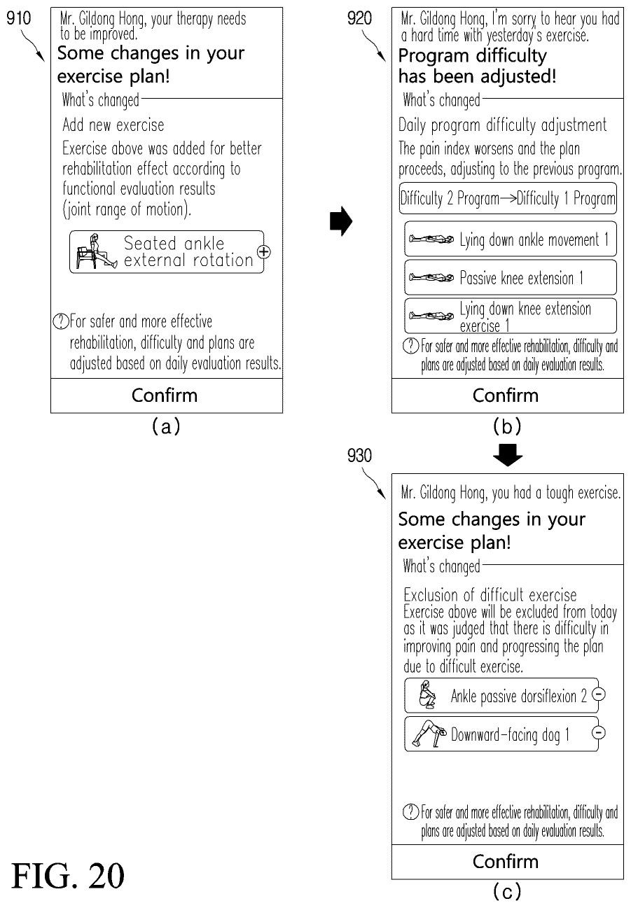

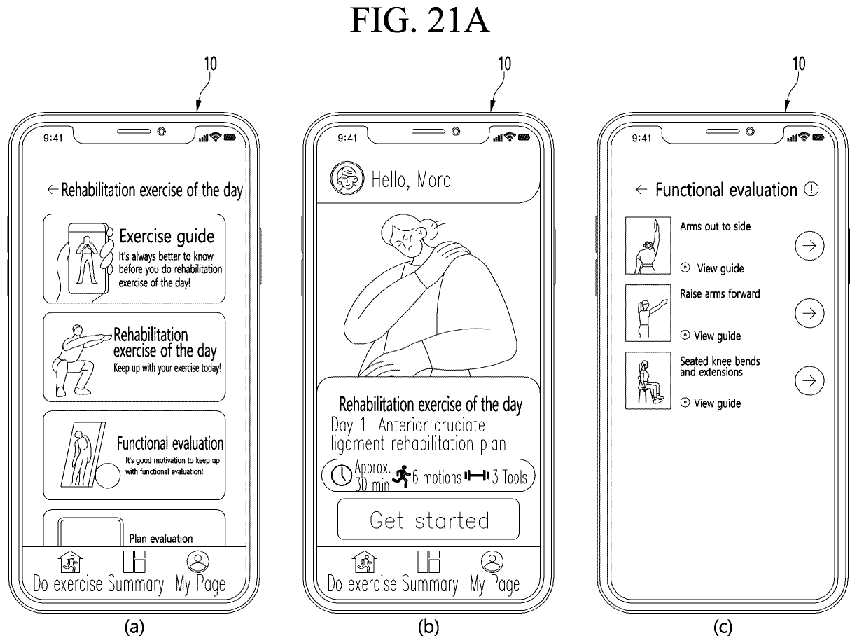

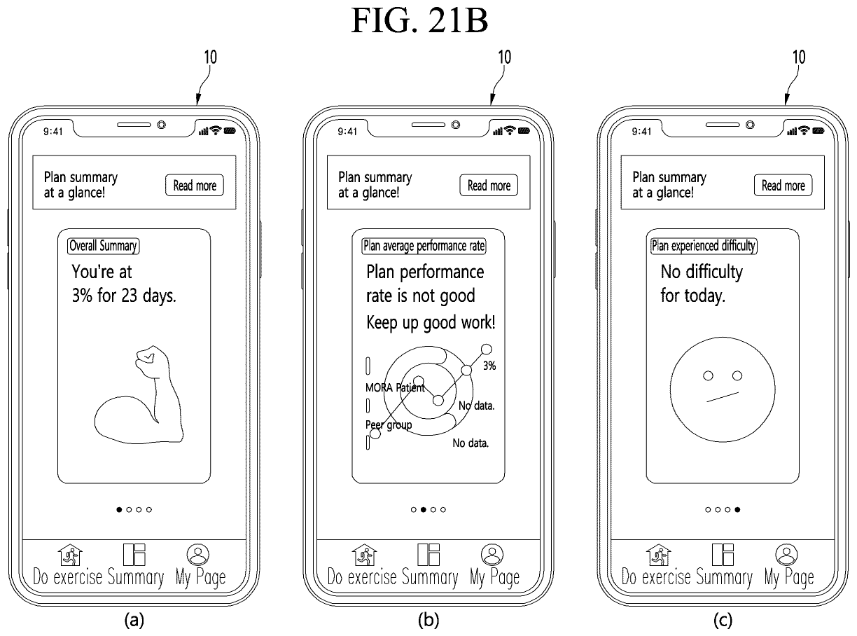

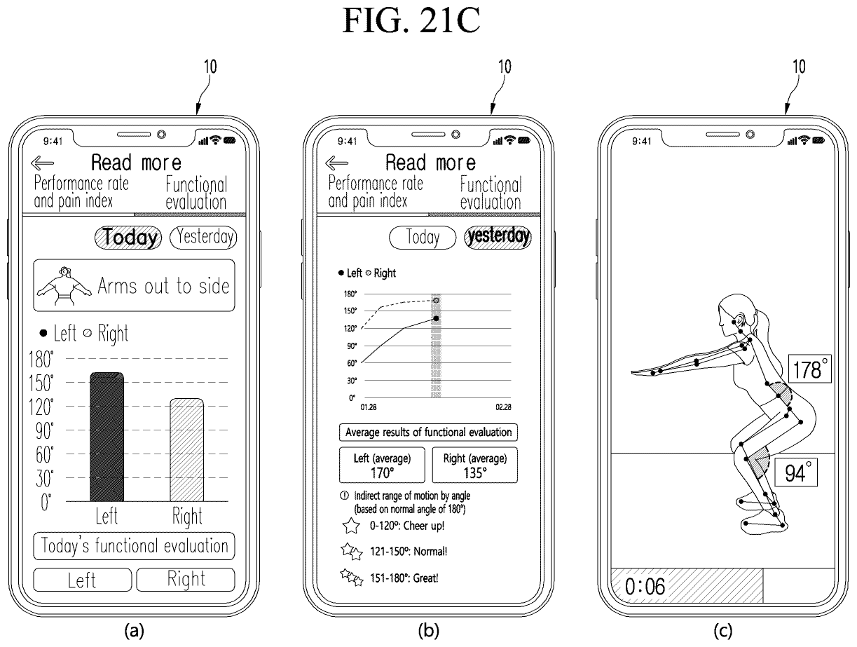

Hereinafter, with reference to the accompanying drawings, a method and system for providing exercise therapy using an artificial intelligence posture estimation model and a motion analysis model according to the present invention will be described in detail. FIG. 11 is a conceptual view for describing a system for providing exercise therapy according to the present invention. FIGS. 12 and 13 are flowcharts for describing a method of providing exercise therapy according to the present invention, FIGS. 14A and 14B are conceptual views for describing a doctor's prescription, FIGS. 15 and 16 are conceptual views for describing a method of analyzing an exercise motion of a patient from an exercise image, FIGS. 17, 18A, 18B, 18C, 18D, 18E, and 18F are conceptual views for describing an artificial intelligence posture estimation model, and FIGS. 19 and 20 are conceptual views for describing an application example that provides a motion analysis result of a user. Further, FIGS. 21A, 21B, and 21C are conceptual views for describing a user environment in which an exercise motion analysis result of a patient is provided.

As illustrated in FIG. 11, a system 1000 for providing exercise therapy according to the present invention is capable of analyzing an exercise motion of a patient in an exercise image received from a patient terminal 10 using an artificial intelligence posture estimation and motion analysis model, and may be configured to include at least one of an application 100 installed on the patient terminal 10 or an artificial intelligence server (or cloud server) 200. Further, the system 1000 for providing exercise therapy according to the present invention may include a posture estimation model and a motion analysis model trained using training data. It is obvious that at least one of the configurations or functions of a system 1000 for providing exercise therapy described below may be included as at least one of the configurations of the system of providing digitally-based musculoskeletal rehabilitation therapy described above. The system for providing digitally-based musculoskeletal rehabilitation therapy may be configured to include at least one of the functions or configurations described below.

The application 100 in the present invention may be installed on the patient terminal 10 to analyze an exercise motion of a patient U suffering from a musculoskeletal disease, and perform a function of providing feedback information based on an analysis result. Accordingly, the application 100 according to the present invention may be referred to as a “digital exercise therapy solution”, “digital rehabilitation therapy solution”, “digital exercise evaluation solution”, “contactless exercise therapy solution”, “contactless rehabilitation therapy solution”, “contactless exercise evaluation solution”, “mobile exercise therapy program”, “mobile rehabilitation therapy program”, “mobile exercise evaluation program”, and “mobile orthopedic rehabilitation assistant (MORA)”.

The application 100 according to the present invention may be installed on the patient terminal 10 to connect the patient U of a musculoskeletal disease with an orthopedic doctor D to perform a role in assisting the patient U in rehabilitation. Hereinafter, for convenience of description, the application 100 installed on the patient terminal 10 will be described to be referred to as an “exercise therapy application”.

Meanwhile, the exercise therapy application 100 according to the present invention may be installed on the patient terminal 10. The patient terminal 10 described in the present invention means an electronic device logged in with a user account of the patient U. For example, the electronic device may include at least one of a smart phone, a cell phone, a tablet PC, a kiosk, a computer, a laptop, a digital broadcasting terminal, a personal digital assistant (PDA), or a portable multimedia player (PMP).

Here, the user account of the patient U may mean an account of the patient U registered in the system 1000 for providing exercise therapy according to the present invention. The user account of the patient U may be understood as a “patient account” or “patient ID” (identification or identification number). In the present invention, the terms “patient”, “patient account (or a user account of a patient)” and “patient terminal” may be used interchangeably.

Meanwhile, the doctor may provide a prescription related to an exercise to the patient U through a doctor terminal 20. In the present invention, the doctor terminal 20 may mean an electronic device logged in with a user account of the doctor D. The user account of the doctor D is an account of the doctor D registered in the system 1000 for providing exercise therapy according to the present invention, which may be understood as a “doctor account” or “doctor ID” (identification or identification number). In the present invention, the terms “doctor”, “doctor account” (or a user account of a doctor), and “doctor terminal” may be used interchangeably.

The doctor D may provide a prescription for the patient U with reference to a user DB 30 that includes the user information of the patient U.

User information (or patient information) of the patient U matched to each patient account may exist in the user DB 30. The user information of the patient U may include various information needed to provide exercise therapy. For example, the user information of the patient U may include at least one of disease information, age information, gender information, surgical history information, exercise plan information, exercise performance information, height information, or weight information of the patient U. However, the user information of the patient described above is just illustrative, and it is obvious that the user information of the patient may include various information necessary to provide exercise therapy to the patient.

Meanwhile, the exercise therapy application 100 described in the present invention is installed on the patient terminal 10 and may analyze an exercise motion of the patient who performed the exercise according to the prescription of the doctor D, through the artificial intelligence posture estimation model and the artificial intelligence motion analysis model, and provide the analysis on the patient terminal 10.

Further, the exercise therapy application 100 may be configured to communicate with the artificial intelligence server 200, and may provide the exercise motion analysis result of the patient analyzed by the artificial intelligence server 200 to the patient terminal 10. The exercise motion analysis result of the patient analyzed by the artificial intelligence server 200 may be generated by at least one of an artificial intelligence motion analysis unit 212 and a rule-based motion analysis unit 213 included in a motion analysis unit 210.

The exercise therapy application 100 is configured to transmit and receive mutual data through wireless communication with the artificial intelligence server 200, and a wireless communication method is not limited. The exercise therapy application 100 according to the present invention may perform communication with the artificial intelligence server 200 using a communication module provided on the patient terminal 10. The communication module provided in the patient terminal 10 may vary.

For example, the communication module provided in the patient terminal 10 may be configured to perform the communication with the artificial intelligence server 200 using at least one of wireless LAN (WLAN), wireless-fidelity (Wi-Fi), wireless-fidelity (Wi-Fi) direct, digital living network alliance (DLNA), wireless broadband (WiBro), world interoperability for microwave access (WiMAX), high speed downlink packet access (HSDPA), high speed uplink packet access (HSUPA), long term evolution (LTE), long term evolution-advanced (LTE-A), fifth generation mobile telecommunication (5G), Bluetooth™ radio frequency identification (RFID), infrared communication (infrared data association (IrDA)), ultra-wideband (UWB), ZigBee, near field communication (NFC), Wi-Fi direct, or wireless universal serial bus (wireless USB) technologies.

Meanwhile, the artificial intelligence server 200 described in the present invention may be a cloud server that performs an exercise motion analysis of the patient U from an exercise image. The artificial intelligence server 200 may perform an analysis of the exercise motion of the patient U using the exercise image received from the exercise therapy application 100. The “artificial intelligence server” described in the present invention may be referred to as an “artificial intelligence exercise therapy server”, “artificial intelligence rehabilitation therapy server”, “digital therapy server”, etc. Hereafter, for convenience of description, this will be described to be referred to as an “artificial intelligence server”.

Meanwhile, at least one of the exercise therapy application 100 or the artificial intelligence server 200 according to the present invention may analyze a relative positional relationship between keypoints P1 and P2 corresponding to a plurality of joint points of the patient U extracted from an exercise image 300 through a posture estimation model 52 (corresponding to the artificial intelligence posture estimation unit 121a in FIG. 11) trained using training data related to the joint points, as illustrated in FIG. 17. The analysis of the relative positions of the keypoints may be performed by the motion analysis unit 120 or 210. In particular, the exercise motion analysis may be performed by one of the artificial intelligence motion analysis unit 122 or 212 and the rule-based motion analysis unit 123 or 213 of the motion analysis unit. One of the artificial intelligence motion analysis unit 122 or 212 or the rule-based motion analysis unit 123 or 213 may be referred to as an artificial intelligence motion analysis model.

Here, the term “joint point” may mean a plurality of joints of the patient U (or a part of the body of the patient U that includes joints).

Further, the term “keypoint” may mean an area corresponding to each of a plurality of joint points of the subject U in the exercise image 300.

Accordingly, in the present invention, the terms “joint point” and “keypoint” may be used interchangeably,

and each of the joint point and keypoint may be described by assigning the same reference numeral “P1, P2”.

The system 1000 for providing exercise therapy may, using the posture estimation model 52, extract the keypoints P1 and P2 corresponding to the joint points from the exercise image of the patient, and analyze the exercise motion of the patient U based on the analysis of the positional relationship between the extracted keypoints P1 and P2. In the present invention, a series of processes for analyzing an exercise motion of a patient from an exercise image using keypoints extracted through the artificial intelligence posture estimation model 52 may be referred to as an “exercise motion analysis process”.

The exercise motion analysis process may be performed by at least one of the exercise therapy application 100 or the artificial intelligence server 200. Specifically, the exercise motion analysis process may include at least one of: i) a first data processing method performed by the exercise therapy application 100, ii) a second data processing method performed by the artificial intelligence server 200, or iii) a third data processing method performed by both the exercise therapy application 100 and the artificial intelligence server 200.

Here, the third data processing method may be performed sequentially or simultaneously in each of the exercise therapy application 100 and the artificial intelligence server 200.

Accordingly, in the present invention, the exercise motion analysis process may be described to be performed in the system 1000 for providing exercise therapy, without distinguishing a physical space and a subject in which the exercise motion analysis process is performed.

Meanwhile, as illustrated in FIG. 17, the exercise motion analysis process may be performed using the keypoints extracted from the artificial intelligence posture estimation model 52. The artificial intelligence posture estimation model 52 may specify or estimate a joint point of a patient from an exercise image through learning on training data specialized for the joint point, and extract a corresponding keypoint.

In the present invention, the training data on which the artificial intelligence posture estimation model 52 is trained may be stored in a database 40, which may also be referred to as a “training data DB”. More details on the training data will be described below.

As illustrated in FIG. 17, the posture estimation server 50 may include at least one of a training unit 51 or a posture estimation model 52. The posture estimation server 50 may be provided inside the system 1000 for providing exercise therapy according to the present invention, or may be configured as an external server. That is, the posture estimation server 50 according to the present invention may be understood as performing a function of performing learning for posture estimation, and not being constrained by a physical space. The details of the posture estimation server 50 are described below along with the training data.

Meanwhile, as illustrated in FIG. 11, the exercise therapy application 100 according to the present invention may include a configuration of at least one of an image receiving unit 110, the motion analysis unit 120, an image processing unit 130, or a control unit 140.

The image receiving unit 110 of the exercise therapy application 100 may be configured to receive an exercise image including an image of a patient exercising from the patient terminal 10 on which the application 100 is installed. Such an exercise image may be photographed by a camera installed on the patient terminal 10. In the present invention, “receiving an exercise image from the patient terminal 10” may be understood to mean that the image receiving unit 110 of the exercise therapy application 100 accesses an exercise image recorded in a memory of the patient terminal 10.

The motion analysis unit 120 of the exercise therapy application 100 may perform an analysis of the exercise motion (or exercise posture) of the patient based on an exercise image received from the patient terminal 10. To this end, the motion analysis unit 120 of the exercise therapy application 100 may be configured to include a configuration of at least one of a keypoint extraction unit 121, the artificial intelligence motion analysis unit 122, or the rule-based motion analysis unit 123. The artificial intelligence motion analysis unit 122 or the rule-based motion analysis unit 123 may be referred to as an “artificial intelligence motion analysis model”.

The keypoint extraction unit 121 may, from the exercise image, extract the keypoints P1 and P2 that is configured in the form of paired x-axis and y-axis coordinate information. In this case, the keypoint extraction unit 121 may extract the keypoints from an image using the artificial intelligence model.

In the present invention, the keypoint extraction using the artificial intelligence model may be described as being performed by the artificial intelligence posture estimation unit 121a included in the keypoint extraction unit 121.

The artificial intelligence posture estimation unit 121a may be referred to as an “artificial intelligence posture estimation model” and may extract a keypoint corresponding to a joint point of a patient from an exercise image using the artificial intelligence model trained for object detection from an image. The artificial intelligence posture estimation model may be a model based on the object detection. For example, the artificial intelligence posture estimation unit 121a may extract keypoints from an exercise image using an object detection artificial intelligence model that ensembles a plurality of bounding boxes. Meanwhile, the artificial intelligence posture estimation unit 121a may use various object detection artificial intelligence models, and the object detection artificial intelligence model described above corresponds to an example.

Further, in the present invention, the artificial intelligence motion analysis unit 122 and the rule-based motion analysis unit 123 may perform an analysis of an exercise motion (or exercise posture) of a patient using at least one of an exercise image received from the patient terminal or a keypoint extracted from the keypoint extraction unit 120.

More specifically, the artificial intelligence motion analysis unit 122 and the rule-based motion analysis unit 123 may: i) perform an analysis of an exercise motion of a patient based on an exercise image, ii) perform an analysis of an exercise motion of a patient based on a keypoint, or iii) perform an analysis of an exercise motion of a patient using both an exercise image and keypoint.

Hereinafter, for convenience of description, a method of analyzing an exercise motion of a patient based on a keypoint will be mainly described. However, it is obvious that the artificial intelligence motion analysis unit 122 and the rule-based motion analysis unit 123 receive an exercise image rather than a keypoint as input data and may perform an exercise motion analysis of a patient directly from the exercise image. Meanwhile, the artificial intelligence motion analysis unit 122 or the rule-based motion analysis unit 123 may also be expressed as the aforementioned “artificial intelligence motion analysis model”.

Meanwhile, the artificial intelligence motion analysis unit 122 may, based on an artificial intelligence model (or the posture estimation model, see reference numeral “52” in FIG. 17) trained to analyze an exercise motion (or exercise posture) of a patient from a keypoint, perform an exercise type classification (or exercise type specification) of an exercise performed by the patient, and an accuracy judgment of the exercise motion.

Furthermore, the rule-based motion analysis unit 123 may, based on rule information defined for analyzing an exercise motion of a patient, perform the exercise type classification (or exercise type specification) of the exercise performed by the patient, and the accuracy judgment of the exercise motion.

Here, the “rule information” is information including various rules used to analyze the exercise motion, which may include, for example, standard joint range of motion information for each exercise motion (or exercise type). The term “rule information” may be used interchangeably with terms such as “reference information” and “standard information”.

Further, the rule information may include, in addition to the range of motion of a joint, various rule information for performing an analysis of at least one of a distance of motion of a joint, a speed (or acceleration) of motion of a joint, a body balance of a subject (corresponding to a patient) included in an exercise image targeted for analysis, a body balance, or a body alignment state (e.g., an axial alignment state of a leg, a spinal alignment state, etc.). The rule-based motion analysis unit 123 may derive various analysis results from the exercise image of the patient targeted for analysis based on the rule information.

In the present invention, an analysis of an exercise motion of a patient from an exercise image may be performed by at least one of the artificial intelligence motion analysis unit 122 or the rule-based motion analysis unit 123.

Specifically, in the present invention, i) an analysis of an exercise motion of a patient may be performed by the artificial intelligence motion analysis unit 122 (a “first analysis performance method”), ii) an analysis of an exercise motion of a patient may be performed by the rule-based motion analysis unit 123 (a “second analysis performance method”), or iii) an analysis of an exercise motion of a patient may be performed by both the artificial intelligence motion analysis unit 122 and the rule-based motion analysis unit 123 (a “third analysis performance method”).

Here, in the third analysis performance method, data processing may be performed sequentially or simultaneously in each of the artificial intelligence motion analysis unit 122 and the rule-based motion analysis unit 123.

Meanwhile, the image processing unit 130 of the exercise therapy application 100 may be configured to overlap or render graphic objects corresponding to the extracted keypoints P1 and P2 on the subject U of the patient included in the exercise image 300. This allows the patient to intuitively recognize the joint points that are being analyzed for the exercise motion of the patient.

The control unit 140 of the exercise therapy application 100 may be configured to perform an overall control of the configurations included in the exercise therapy application 100. The control unit 140 of the exercise therapy application 100 may control the configurations of the exercise therapy application 100 using a central processing unit (CPU) of the patient terminal 10, and may further perform a control of the configurations (e.g., a communication module, a camera module, a sensing module, an output module (e.g., a display, a speaker), and an input module (e.g., a touch screen, a microphone) provided in the patient terminal 10.

Meanwhile, as illustrated in FIG. 11, the artificial intelligence server 200 is a cloud server configured to perform exercise posture of a patient using the artificial intelligence posture estimation model, and may be configured to include a configuration of at least one of a motion analysis unit 210 or a control unit 220.

The motion analysis unit 210 of the artificial intelligence server 200 may perform an analysis of the exercise motion (or exercise posture) of the patient based on an exercise image received from the patient terminal 10.

The motion analysis unit 210 of the artificial intelligence server 200 may receive an exercise image of a patient from the exercise therapy application 100, and the receiving of the exercise image may be performed by a communication unit (or communication module) of the artificial intelligence server 200.

The motion analysis unit 210 of the artificial intelligence server 200 may be configured to include a configuration of at least one of a keypoint extraction unit 211, an artificial intelligence motion analysis unit 212, or a rule-based motion analysis unit 213. The artificial intelligence motion analysis unit 212 or the rule-based motion analysis unit 213 may be referred to as an “artificial intelligence motion analysis model”.

Each of the keypoint extraction unit 211, artificial intelligence motion analysis unit 212, and rule-based motion analysis unit 213 included in the artificial intelligence server 200 may perform the same functions as the keypoint extraction unit 121, artificial intelligence motion analysis unit 122, and rule-based motion analysis unit 123 of the previously described exercise therapy application 100. In this regard, a detailed description will be omitted.

The control unit 220 of the artificial intelligence server 200 may be configured to perform an overall control of the configurations included in the artificial intelligence server 200.

Hereinafter, an exercise motion analysis process for analyzing an exercise motion of the patient U from an exercise image and providing an exercise motion analysis result will be described using the configuration above of the system 1000 for providing exercise therapy according to the present invention.

As illustrated in FIG. 12, an exercise prescription for the patient U may be made in the doctor terminal 20 (S210), and the system 1000 for providing exercise therapy may, based on the exercise prescription made for the patient in the doctor terminal 20, receive prescription information on the exercise prescription from the doctor terminal 20.

The system 1000 for providing exercise therapy may, based on the prescription information being received from the doctor terminal 20, allocate, to a patient account, an exercise plan that includes at least one prescribed exercise according to the prescription information. The allocated exercise plan may be transmitted to the patient terminal 10 (S220).

The system 1000 for providing exercise therapy may include a communication unit that performs communication with at least one of the patient terminal 10, the doctor terminal 20, the user DB 30, or the database 40. For example, the communication unit may perform the communication using at least one of wireless LAN (WLAN), wireless-fidelity (Wi-Fi), wireless-fidelity (Wi-Fi) direct, digital living network alliance (DLNA), wireless broadband (WiBro), world interoperability for microwave access (WiMAX), high speed downlink packet access (HSDPA), high speed uplink packet access (HSUPA), long term evolution (LTE), long term evolution-advanced (LTE-A), fifth generation mobile telecommunication (5G), Bluetooth™ radio frequency identification (RFID), infrared communication (infrared data association (IrDA)), ultra-wideband (UWB), ZigBee, near field communication (NFC), Wi-Fi direct, or wireless universal serial bus (wireless USB) technologies.

Meanwhile, an exercise image of the patient performing the prescribed exercise included in the exercise plan may be photographed in the patient terminal 10 (S230). The exercise therapy application 100 may activate a camera provided on the patient terminal 10 to control the exercise image to be photographed.

The exercise image photographed at the patient terminal 10 may be used as analysis target data (or exercise image targeted for analysis) for an exercise motion analysis of the patient by the system 1000 for providing exercise therapy.

As described above, the exercise motion analysis process may be performed in at least a portion of the exercise therapy application 100 installed on the patient terminal 10 or the artificial intelligence server 200, and may be described in the present invention as being performed in the system 1000 for providing exercise therapy, without separately distinguishing a physical space where the exercise motion analysis process is performed and a subject.

Meanwhile, the system 1000 for providing exercise therapy may extract the keypoints P1 and P2 corresponding to a plurality of joint points from the exercise image. The extracting of the keypoints P1 and P2 may be performed by at least a portion of the keypoint extraction unit 121 included in the exercise therapy application 100 or the keypoint extraction unit 121 included in the artificial intelligence server 200.

The system 1000 for providing exercise therapy may perform an analysis of a relative positional relationship between the extracted keypoints P1 and P2 (S250). Further, the system 1000 for providing exercise therapy may analyze the exercise motion of the patient U based on the analysis of the positional relationship between the keypoints P1 and P2 (S260). This exercise motion analysis may be performed by at least a portion of the motion analysis unit 120 of the application 100 or the motion analysis unit 210 of the artificial intelligence server 200.

Further, the system 1000 for providing exercise therapy may provide an exercise motion analysis result of the patient U as feedback information to the patient terminal 10 and as monitoring information to the doctor terminal 20 (S270).

As described above, the system 1000 for providing exercise therapy may perform an overall control of the exercise motion analysis process, which, in the present invention, may be understood to be performed by the control unit of the system 1000 for providing exercise therapy. That is, the control unit of the system 1000 for providing exercise therapy is a concept that includes the control unit 140 of the exercise therapy application 100 and the control unit 220 of the artificial intelligence server 200, which may perform an overall control of the system 1000 for providing exercise therapy.

Hereinafter, the exercise motion analysis process performed by system 1000 for providing exercise therapy will be described in more detail.

In the present invention, a process of receiving, from the doctor terminal, prescription information related to an exercise for a patient may proceed (S310, see FIG. 13).

As illustrated in FIGS. 14A and 14B, system 1000 for providing exercise therapy may provide an exercise prescription page (or exercise assignment page) that includes a prescription function related to an exercise for a patient, on the doctor terminal 20 logged in with a doctor account. In the present invention, the term “exercise prescription” may be used interchangeably with the term “exercise assignment”.

The system 1000 for providing exercise therapy may provide, on the doctor terminal 20, an exercise prescription page for each patient account, such that a prescription may be made for a specific patient U account among the patient accounts matched to the doctor account.

For example, in the present invention, assume that a specific doctor D account is matched with a first patient account (e.g., patient account “Wooyoung Kim”) and a second patient account (e.g., patient account “Sohee Kim”). The system 1000 for providing exercise therapy may provide, on the doctor terminal 20, an exercise prescription page corresponding to the first patient account (e.g., patient account “Wooyoung Kim”) based on an exercise prescription request for the first patient account being received from the doctor terminal 20.

The system 1000 for providing exercise therapy may receive, based on a user selection (or user input) made on the exercise prescription page corresponding to the specific patient, prescription information for the specific patient from the doctor terminal 20. The prescription information may include a variety of information for prescribing an exercise to a patient. For example, the prescription information may include at least one of: i) information on at least one exercise motion that should be included in an exercise plan (e.g., “Calf stretch against a wall”, “Seated rolling ball sole massage”), ii) difficulty information on the exercise motion, iii) duration information on the exercise motion, iv) information on the number of times to perform the exercise motion, v) schedule information on performing the exercise motion, vi) body information matched to the exercise motion (e.g., “Ankle” and “Knee”), or vii) caution information (e.g., “After the exercise, please apply an ice pack”) (see FIG. 14A and (a) of FIG. 14B).

The system 1000 for providing exercise therapy may receive prescription information for the specific patient U from the doctor terminal 20 based on the prescription information on the specific patient being input (or selected) on the exercise prescription page corresponding to the specific patient. In this case, guidance information may be output on the doctor terminal 20 to guide that a prescription has been made for the specific patient (see (b) of FIG. 14B).

Meanwhile, in the present invention, based on the prescription information, a process of allocating an exercise plan including at least one prescribed exercise to a patient account may proceed (S320, see FIG. 13).

As illustrated in FIG. 15, the system 1000 for providing exercise therapy may, based on the prescription information on the specific patient U, allocate an exercise plan E including at least one prescribed exercise to the specific patient account, and provide the allocated exercise plan (e.g., “Patellofemoral osteoarthritis digital therapy”, E) to the patient terminal 10 logged in with the specific patient account.

Herein, the term “prescribed exercise” may be understood as an exercise motion that is specified and allocated to a patient account based on prescription information among a plurality of exercise motions (or exercise types) included in the system 1000 for providing exercise therapy. Accordingly, in the present invention, the term “prescribed exercise” may be used interchangeably with the term “exercise motion”. Further, in the present invention, the term “exercise plan” may be used interchangeably with the term “digital therapy”.

The system 1000 for providing exercise therapy may, based on receiving a request to provide an exercise plan allocated to a specific patient account from a patient terminal 10 logged in with the specific patient account, provide an exercise page, on the patient terminal 10, associated with an exercise guide image providing function, for the patient to perform a prescribed exercise included in the exercise plan.

As illustrated in FIG. 15, the exercise page may include an exercise list L, in which the exercise list L may include items V1 to V6 corresponding to exercise guide images for each of a plurality of prescribed exercises (e.g., “Leg upright” and “Standing knee bend”) included in the exercise plan allocated to the specific account.

The system 1000 for providing exercise therapy may, when the exercise plan includes a specific prescribed exercise (e.g., “Leg upright”) having a plurality of exercise sets, control such that the items V1 to V3 corresponding to the exercise guide image of the specific prescribed exercise are included in an exercise list L by the number of the sets (e.g., “ ”).

Meanwhile, the system 1000 for providing exercise therapy may, based on receiving a request to start an exercise from the patient terminal 10, control such that a plurality of exercise guide images are played sequentially on the patient terminal 10 based on a sequence of items V1 to V6 included in the exercise list L.

Meanwhile, in the present invention, a process of receiving, from the patient terminal, an exercise image that photographs an exercise according to the prescribed exercise may be performed (S330, see FIG. 13).

As illustrated in FIG. 16, the system 1000 for providing exercise therapy may control a camera provided on the patient terminal 10 to photograph an exercise image of the patient U based on the exercise guide image being played on the patient terminal 10.

The exercise therapy application 100 installed on the patient terminal 10 may control an activation state of a camera provided on the patient terminal 10 from an inactive state to an active state, such that the camera is controlled to photograph an exercise image of the patient U performing an exercise motion according to the exercise guide image.

As illustrated in FIG. 16A, the exercise therapy application 100 may, in order to detect a subject U corresponding to the patient from the exercise image being photographed through the camera, output a guidance message (e.g., “Please stand inside the screen”) on the patient terminal 10 such that the entire body of the patient is included within a specific area of the exercise image (or a display of the patient terminal).

The exercise therapy application 100 may, based on the subject U corresponding to the entire body of the patient being included within the specific area, detect the subject U from an image 300 using an object detection algorithm.

The exercise therapy application 100 may use a variety of object detection algorithms. For example, the exercise therapy application 100 may use an algorithm (weighted box fusion (WBF)) that ensembles a plurality of bounding boxes. However, it is obvious that the exercise therapy application 100 is not limited to the object detection algorithm described above, but may utilize various object detection algorithms that are capable of detecting an object corresponding to the subject U from the training target exercise image 300.

Further, the exercise therapy application 100 may, based on the subject U corresponding to the entire body of the patient being detected within the specific area, photograph, through the camera, an exercise image of the patient performing an exercise motion according to the prescribed exercise.

In this case, the exercise therapy application 100 may photograph the patient performing the prescribed exercise in a state where an exercise guide image corresponding to the prescribed exercise allocated to the patient is played.

Further, the exercise therapy application 100 may control an exercise image that is photographed by the camera of the patient terminal 10 to be matched to an exercise plan (or each of a plurality of prescribed exercises included in the exercise plan) and recorded in the memory of the patient terminal 10.

Meanwhile, in the present invention, a process of extracting a keypoint corresponding to each of a plurality of preset joint points from the exercise image may proceed (S340, see FIG. 13).

In the present invention, keypoints P1 and P2 corresponding to joint points P1 and P2 preset from the exercise image may be extracted by at least a portion of the exercise therapy application 100 or the artificial intelligence server 200. As described above, the extraction of the keypoints P1 and P2 may be performed by: i) the exercise therapy application 100, ii) the artificial intelligence server 200, or iii) both the exercise therapy application 100 and the artificial intelligence server 200. Hereinafter, it will be described that the extraction of keypoints P1 and P2 is performed by the system 1000 for providing exercise therapy, without separately distinguishing the subject that performs the extraction.

The system 1000 for providing exercise therapy may extract, from the exercise image 300, areas corresponding to predefined (or preset) joint points of the plurality of joint points of the patient as keypoints P1 and P2.

Here, the term “joint point” may mean a plurality of joints of the patient U (or a part of the body of the patient U that includes joints).

Further, the term “keypoint” may mean an area corresponding to each of a plurality of joint points of the subject U in the exercise image 300.

In the present invention, the terms “joint point” and “keypoint” may be used interchangeably, and each of the joint point and keypoint may be described by assigning the same reference numeral “P1 or P2”.

Meanwhile, the human body is made up of more than 200 bones, and a joint is apart where bone connects to each other, and the human body may consist of a plurality of joints.

In the present invention, among the plurality of joint points constituting the human body, joint points that are targeted as keypoints are predesignated and may exist as joint point definition information 500. For example, in the joint point definition information 500, a first joint point P1 corresponding to a center of the head 510 and a second joint point P2 corresponding to a center of the neck 520 may exist to be predefined (see FIG. 18D).

The system 1000 for providing exercise therapy may, based on the posture estimation model 52 trained using a training data set including position information on preset joint points, extract the keypoints P1 and P2 corresponding to the joint points from the exercise image 300.

In this case, the system 1000 for providing exercise therapy may, based on the position information of each of the joint points preset by the posture estimation model being extracted in the form of paired x-axis and y-axis coordinate information, specify the positions of the keypoints P1 and P2 in the exercise image 300.

Meanwhile, the system 1000 for providing exercise therapy may, based on whether the joint point is visible in the exercise image 300, extract (or specify) the keypoints P1 and P2 corresponding to the joint points according to any one process of a first keypoint extraction process or a second keypoint extraction process.

In the present invention, whether a joint point is visible may be understood to mean whether the joint point is visible in the exercise image 300.

The system 1000 for providing exercise therapy may judge that the joint point of the exercise image 300 is visible when the exercise image 300 includes a body part of the subject U that corresponds to the joint point.

The system 1000 for providing exercise therapy may, when a specific joint point is visible in the exercise image 300, extract a keypoint corresponding to the specific joint point according to the first keypoint extraction process.

Specifically, the system 1000 for providing exercise therapy may specify a visible joint point of the subject U that is visible in the exercise image 300 among a plurality of preset joint points. For example, the system 1000 for providing exercise therapy may, when a first joint point and a second joint point of the plurality of preset joint points are visible in the exercise image, specify the first joint point and the second joint point as visible joint points.

Further, the system 1000 for providing exercise therapy may extract the specified visible joint point as a keypoint.

In this case, the system 1000 for providing exercise therapy may extract position information on an area (or pixel) corresponding to the visible joint point in the exercise image to extract a keypoint corresponding to the visible joint point. For example, the system 1000 for providing exercise therapy may extract position information on a visible joint point using the object detection algorithm to extract a keypoint corresponding to the visible joint point.

In the present invention, the position information of the visible joint point extracted according to the first keypoint extraction process may be described to be referred to as a “first type of information (first type of position information)” or “substantial position information”.

In contrast, the system 1000 for providing exercise therapy may judge that the joint point of the exercise image 300 is invisible when the exercise image 300 does not include a body part of the subject U that corresponds to the joint point.

The system 1000 for providing exercise therapy may, when a specific joint point is invisible in the exercise image 300, predict and extract a keypoint corresponding to the specific joint point using the posture estimation model 52 according to the second keypoint extraction process.

The system 1000 for providing exercise therapy may, based on the posture estimation model 52, predict position information of an invisible joint point of the subject U that is not visible in the exercise image 300 among the plurality of preset joint points. In this case, the posture estimation model 52 may predict the position information of the invisible joint point based on the position information of the visible joint point.

In the present invention, the position information of the joint point extracted according to the second keypoint extraction process may be described to be referred to as a “second type of information (second type of position information)” or “expected position information”.

The system 1000 for providing exercise therapy may extract (or specify) the keypoint corresponding to the invisible joint point by matching the predicted position information on the invisible joint point to the keypoint corresponding to the invisible joint point.

As described above, in the present invention, the keypoints P1 and P2 corresponding to the joint points may be extracted (or specified) in the exercise image 300 according to different processes, based on the posture estimation model that has been performed training on the position information of the joint points, depending on whether the predefined joint points are visible. Therefore, in the present invention, it is also possible to analyze invisible joint points that are not visible in the exercise image.

Meanwhile, the system 1000 for providing exercise therapy may extract the keypoints P1 and P2 from the exercise image in real time in conjunction with the exercise image being photographed on the patient terminal 10. Further, the system 1000 for providing exercise therapy may provide the extracted keypoints P1 and P2 on the patient terminal 10 in real time such that the patient may intuitively recognize the joint points being analyzed for the exercise motion.

Specifically, as illustrated in FIGS. 16B and 16C, the system 1000 for providing exercise therapy may output the exercise image 300 in real time on the patient terminal 10 in conjunction with the exercise image 300 being photographed on the patient terminal 10. Further, the system 1000 for providing exercise therapy may provide a graphic object corresponding to the extracted keypoint P1 or P2 that overlaps an area of the subject U corresponding to the preset joint point.

Data processing to provide the keypoint graphic object overlapping the exercise image 300 may be performed by the image processing unit 130 of the exercise therapy application 100. The image processing unit 130 may render each of the graphic objects corresponding to the extracted keypoints P1 and P2 on the area of the subject U corresponding to the joint points P1 and P2 matched to the keypoints P1 and P2.

Further, the image processing unit 130 may, when a position of the preset joint point changes as the patient performs an exercise motion, provide a keypoint graphic object overlapping an area of the subject U corresponding to the changed joint point. That is, the image processing unit 130 may allow the keypoint graphic object to overlap the area corresponding to the joint point in the exercise image such that the position of the joint point that changes in real time is reflected.

Meanwhile, in the present invention, a process of analyzing a relative positional relationship between keypoints, from the keypoints extracted through the posture estimation model trained using training data related to the joint points, and analyzing an exercise motion of the patient for the prescribed exercise based on the analysis of the relative positional relationship may proceed (S350, see FIG. 13). The analysis of the relative positions of the keypoints may be performed by the motion analysis unit 120 or 210. In particular, the exercise motion analysis may be performed by one of the artificial intelligence motion analysis units 122 and 212 and the rule-based motion analysis units 123 and 213 of the motion analysis unit.

As illustrated in FIG. 16D, the exercise therapy application 100 may provide, on the patient terminal 10, guidance information to guide an analysis progression (e.g., “Calculating the result” or “Mr. Cheolsoo Kim, I will provide you with the exercise motion analysis result”) to guide the patient through an exercise motion analysis. Hereinafter, a method of analyzing patient exercise motion will be described in detail.

The system 1000 for providing exercise therapy may analyze the relative positional relationship between the keypoints P1 and P2 corresponding to each of the plurality of preset joint points using the keypoints extracted from the posture estimation model trained using the training data.

The system 1000 for providing exercise therapy may analyze a relative position between the keypoints P1 and P2 corresponding to each of the plurality of preset joint points, using both the keypoints corresponding to visible joint points and the keypoints corresponding to invisible joint points.

Here, the “relative position between keypoints” may be understood as a position of one keypoint (e.g., a first keypoint, “P1”) relative to another keypoint (e.g., a second keypoint, “P2”), between at least two keypoints P1 and P2.

Hereinafter, for convenience of description, a keypoint corresponding to a visible joint point will be referred to as a “first type keypoint” and a keypoint corresponding to an invisible joint point will be referred to as a “second type keypoint”.

The system 1000 for providing exercise therapy may perform an analysis of at least one of: i) a relative positional relationship between a plurality of first type keypoints, ii) a relative positional relationship between a first type keypoint and a second type keypoint, or iii) a relative positional relationship between a plurality of second type keypoints.

In this case, the system 1000 for providing exercise therapy may, based on a type of prescribed exercise performed by the patient, analyze a relative positional relationship between some associated keypoints of the plurality of joint points.

For example, the system 1000 for providing exercise therapy may analyze a relative positional relationship between keypoints corresponding to each of a first joint point and a second joint point of the plurality of joint points when the patient performs a prescribed exercise according to a first exercise type.

In another example, the system 1000 for providing exercise therapy may analyze a relative positional relationship between keypoints corresponding to each of the first joint point and a third joint point of the plurality of joint points when the patient performs a prescribed exercise according to a second exercise type that is different from the first exercise type. This relative positional relationship may consequently be used for motion analysis.

The results of the motion analysis performed in the system 1000 for providing exercise therapy according to the present invention may vary widely. For example, the system 1000 for providing exercise therapy may, from the extracted keypoints or images, perform an analysis of at least one of a range of motion of a joint, a distance of motion, a speed (or acceleration) of motion of a joint, a body balance of a subject (corresponding to a patient) included in an exercise image targeted for analysis, a body balance, and a body alignment state (e.g., an axial alignment state of a leg, a spinal alignment state, etc.). Meanwhile, the system 1000 for providing exercise therapy according to the present invention may analyze a relative positional relationship between keypoints based on rule information related to a prescribed exercise.

Here, the rule information may be understood as information for which rules are predefined to analyze a relative positional relationship between keypoints.

The system 1000 for providing exercise therapy may analyze an exercise motion of a patient by judging whether a relative positional relationship between keypoints satisfies the rule information. Hereinafter, a method of analyzing a range of motion of a joint based on the relative positional relationship of keypoints and the rule information will be described as an example. However, the content described below is only one embodiment of analyzing a motion of a patient based on the relative positional relationship of keypoints and the rule information, and in the present invention, various motions of a patient may be analyzed based on the relative positional relationship of keypoints and the rule information.

The range of motion of a joint analyzed in the system 1000 for providing exercise therapy according to the present invention will be described in more detail below. The system 1000 for providing exercise therapy may, based on the rule information on standard range of motion of a joint related to a prescribed exercise, perform an analysis of a range of motion of a patient's joint depending on a relative positional relationship between keypoints.

Further, the system 1000 for providing exercise therapy may, based on the rule information related to the prescribed exercise, analyze a relative positional relationship between the keypoints. Further, the system 1000 for providing exercise therapy may analyze the exercise motion of the patient by judging whether the relative positional relationship between the keypoints satisfies the rule information.

The system 1000 for providing exercise therapy may extract, from a plurality of consecutive frames related to a specific prescribed exercise, a relative positional relationship between associated keypoints matched to the specific prescribed exercise, and obtain (or calculate) a range of motion of the patient's joint for the specific prescribed exercise using the extracted relative positional relationship.

Specifically, assume that an exercise image is configured with a plurality of frames of a first type corresponding to a first prescribed exercise and a plurality of frames of a second type corresponding to a second prescribed exercise.

The system 1000 for providing exercise therapy may, when an exercise motion analysis of a patient for the first prescription exercise of the first prescription exercise and the second prescription exercise is performed, analyze the exercise motion of the patient using keypoints extracted from the plurality of frames having the first type.

In contrast, the system 1000 for providing exercise therapy may, when an exercise motion analysis of a patient for the second prescription exercise is performed, analyze the exercise motion of the patient using keypoints extracted from the plurality of frames having the second type.

That is, the system 1000 for providing exercise therapy may analyze a keypoint positional relationship for consecutive movements (or posture) to obtain (or calculate) a range of motion for an exercise of the patient for a specific prescribed exercise.

Hereinafter, for convenience of description, a plurality of consecutive frames (i.e., a plurality of frames having a first type) corresponding to a specific prescribed exercise (e.g., the first prescribed exercise) will be referred to as a “first analysis target frame” and a “second analysis target frame,” depending on the temporal sequence in which the frames are formed.

Here, it may be understood that the first analysis target frame is a frame formed temporally before, and the second analysis target frame is a frame formed temporally after.

The system 1000 for providing exercise therapy may extract keypoints from each of the first analysis target frame and the second analysis target frame.

A first analysis target keypoint group corresponding to each of the plurality of joint points may be extracted from the first analysis target frame, and a second analysis target keypoint group corresponding to each of the plurality of joint points may be extracted from the second analysis target frame.

The system 1000 for providing exercise therapy may analyze a “first positional relationship” between keypoints included in the first analysis target keypoint group and perform a first motion analysis of the subject U included in the first analysis target frame. In addition, the system 1000 for providing exercise therapy may analyze a “second positional relationship” between keypoints included in the second analysis target keypoint group and perform a second motion analysis of the subject U included in the second analysis target frame.

The system 1000 for providing exercise therapy may, based on the first keypoint positional relationship and the second keypoint positional relationship, obtain (extract or calculate) a range of motion of a patient's joint for a specific prescribed exercise.

In this case, the system 1000 for providing exercise therapy may obtain the range of motion of the patient's joint in consideration of at least one of age information, gender information, height information, weight information, surgery history information, or musculoskeletal disease information of the patient, with reference to the user DB 30.

Meanwhile, the system 1000 for providing exercise therapy may judge whether the obtained range of motion for the exercise of the patient satisfies the standard range of motion of a joint corresponding to the rule information related to the specific prescribed exercise. In the present invention, the rule-based analysis of the range of motion for the exercise of the patient may be performed by the rule-based motion analysis unit 213 of the artificial intelligence server 200 (see FIG. 11), but the analysis is not limited to being performed by the rule-based motion analysis unit 213.

In the present invention, there may be rule information on the standard range of motion of a joint for each of a plurality of exercise types. This rule information may include information on different standard range of motion of a joint by age, gender, height, weight, and musculoskeletal disease.

The system 1000 for providing exercise therapy may compare a patient's range of motion of a joint for a specific prescribed exercise to the standard range of motion of a joint for the specific prescribed exercise included in the rule information, and judge whether the patient's range of motion of a joint satisfies the standard range of motion of a joint.

The system 1000 for providing exercise therapy may, based on a judgment result, provide an analysis result of the exercise motion of the patient to the patient terminal 10 as feedback for the prescribed exercise.

Meanwhile, in the present invention, a process of transmitting the analysis result of the exercise motion of the patient to the patient terminal may be performed (S360, see FIG. 13).

The system 1000 for providing exercise therapy may provide a motion analysis result in a variety of ways in order for the patient to intuitively recognize the analysis result of the exercise motion and increase the compliance of the patient with the exercise.

The system 1000 for providing exercise therapy may provide graphic objects corresponding to the keypoints P1 and P2 overlapping the exercise image in real time in a state where the exercise image 300 is being photographed on the patient terminal 10 (see FIG. 16).

In this case, the system 1000 for providing exercise therapy may position information on the range of motion of the patient's joint, around the keypoints P1 and P2 related to the range of motion of the joint.

Further, the system 1000 for providing exercise therapy may provide keypoint graphic objects (or graphic objects corresponding to positional relationship between keypoints) having different visual appearances overlapping the exercise image to enable a patient to recognize whether the range of motion of the patient's joint satisfies the standard range of motion of a joint.

Further, the visual appearances of the graphic objects overlapping the exercise image may be configured to be different depending on whether the relative positional relationship between the extracted keypoints satisfies the rule information.

For example, when the range of motion of the patient's joint satisfies the standard range of motion of a joint, a graphical object A having a first visual appearance may overlap the exercise image 300. In contrast, when the range of motion of the patient's joint does not satisfy the standard range of motion of a joint, a graphic object B having a second visual appearance different from the first visual appearance may overlap the exercise image 300.

Further, the system 1000 for providing exercise therapy may, based on keypoints extracted from each of a plurality of frames constituting the exercise image 300, provide an evaluation score of the patient for the prescribed exercise (e.g., “Mr. Wooyoung Kim's squatting posture is 70 points”) as the motion analysis result.

Meanwhile, in the present invention, the exercise therapy application 100 installed on the patient terminal 10 and the artificial intelligence server 200 may perform an analysis of the exercise motion and generate an exercise motion analysis result, respectively.

For example, the exercise therapy application 100 may allow graphical objects corresponding to the keypoints P1 and P2 to overlap the exercise image in real time to generate a first result analysis.

In another example, the artificial intelligence server 200, which is configured as a cloud server, may generate, based on keypoints extracted from each of the plurality of frames constituting the exercise image, an evaluation score of the patient for the prescribed exercise as a second analysis result.

The system 1000 for providing exercise therapy may provide, on the patient terminal 10, an analysis result of an exercise motion of the patient, including the first analysis result generated by the exercise therapy application 100 and the second analysis result generated by the artificial intelligence server 200.

Meanwhile, the system 1000 for providing exercise therapy may transmit an exercise motion analysis result of the patient to the doctor terminal 20. Both the first analysis result and the second analysis result may be provided to the doctor terminal 20.

As such, in the present invention, various user environments related to the provision of an analysis result are provided such that a patient may intuitively recognize the analysis result for an exercise motion. Another embodiment related to the provision of an analysis result will be described below.

Meanwhile, as illustrated in FIG. 17, the present invention is directed to analyzing an exercise motion of the patient U included in the exercise image 300 based on the exercise image 300 received from the patient terminal 10, and providing an analysis result. In particular, the present invention relates to a method of processing and learning a training data set centered on important joint points to analyze an exercise motion of a patient based on artificial intelligence.

Hereinafter, training data on which the posture estimation model of the present invention is trained will be described in detail.

As illustrated in FIG. 17, the database 40 is a storage where the training data set is stored, which may be provided on the system 1000 for providing exercise therapy according to the present invention itself or may be configured as an external storage (or an external DB). It may be understood that the database 40 according to the present invention is sufficient to be the space in which the training data set is stored, and is not limited by a physical space.

The present invention may be configured to include at least one of the database 40, the posture estimation server 50, or the system 1000 for providing exercise therapy.

In the database 40, training data for training the posture estimation model 52 may be stored as a training data set.

As illustrated in FIG. 18B, a training data set 400 in the present invention may be configured with a plurality of data groups 410 to 450, each corresponding to different information attributes 410a to 450a. The information contained in each of the plurality of data groups 410 to 450 may be configured by being extracted from the exercise image 300 including the subject U performing an exercise motion.

Here, the term “exercise image 300” refers to an image (or motion image) that photographs a process in which a user performs an exercise motion, as illustrated in FIG. 18A, which may include at least a portion of the body of the user U.

In the present invention, a user object included in an exercise image 300 may be described to be referred to as “subject U”. In the present invention, the term “subject U” may mean a user or a portion of the body of the user who is exercising in the exercise image. Accordingly, the terms “subject” and “user” may be used interchangeably and may be described by assigning the same reference numeral “U”.

Meanwhile, the “exercise image 300” described in the present invention may include an “exercise image targeted for analysis” and a “training target exercise image”.

It may be understood that the “exercise image targeted for analysis” is an exercise image targeted for posture estimation analysis of the subject U, and the “training target exercise image” is an exercise image targeted for machine learning for the posture estimation model. Here, the pose estimation analysis may mean extracting keypoints from an image.

The training unit 51 may be configured to perform training for the posture estimation model based on the training target exercise image 300. The training unit 51 may train the posture estimation model using the training data.

As illustrated in (a) of FIG. 18B, the training unit 51 may detect the subject U in the training target exercise image 300, and extract various training data used for estimating the exercise posture from the detected subject U. This training data may be used interchangeably with the terms “information”, “data”, or “data value”. Meanwhile, the extraction of training data may also be performed by means other than the training unit 51.

The training unit 51 may use an algorithm for various object detections to detect the subject U from the training target exercise image 300. For example, the training unit 51 may use an algorithm (weighted box fusion (WBF)) that ensembles a plurality of bounding boxes. However, it is obvious that the training unit 51 is not limited to the object detection algorithm described above, but may utilize various object detection algorithms that are capable of detecting an object corresponding to the subject U from the training target exercise image 300.

The training unit 51 may classify the extracted training data into one of the plurality of data groups 410 to 450, corresponding to each of the different plurality of information attributes 410a to 450a.

The different plurality of information attributes 410a to 450a described in the present invention may exist to be predefined, as illustrated in (b) of FIG. 18B. Further, the plurality of data groups 410 to 450 corresponding to each of the plurality of information attributes 410a to 450a may include training data corresponding to the predefined information attributes.

For example, i) the data group 410 corresponding to the first information attribute 410a may include the joint point position information on the subject U, and ii) the data group 420 corresponding to the second information attribute 420a may include information representing whether the joint point of the subject U is visible. Further, iii) the data group 430 corresponding to the third information attribute 430a may include information on the photographing direction of the subject U, iv) the data group 440 corresponding to the fourth information attribute 440a may include information on an exercise code that distinguishes an exercise motion (or an exercise type) performed by the subject U, and v) the data group 450 corresponding to the fifth information attribute 450a may include size and center position information on a bounding box for the subject U.

Here, the “joint point P1 or P2” may mean a joint of the user or an area corresponding to a joint of the subject U in the exercise image 300.

The training unit 51 may generate (constitute) a data set for the training target exercise image 300 by associating the plurality of data groups 410 to 450 extracted from the training target exercise image 300 with each other. Further, the training unit 51 may store the generated training data set 400 in the database 40. The database 40 may be built as a database 40 for the posture estimation model 52, based on the training data set 400 generated by the training unit 51 being stored therein.

Further, the training unit 51 may perform training for the posture estimation model 52 based on the training data set 400 that exists in the database 40. As described above, the training data set 400 may include the position information of the joint points.

The posture estimation model 52 is a posture estimation model trained using a training data set (Data set) including position information for a joint point, and may estimate an exercise posture of the subject U from an exercise image targeted for analysis.

Meanwhile, the posture estimation model 52 may extract keypoints corresponding to joint points of the subject from the exercise image 300 using the training data set 400 generated by the training unit 51, and at least one of the artificial intelligence motion analysis unit 122 or 212 and the rule-based motion analysis unit 123 or 213 may analyze an exercise motion of the subject in the exercise image 300 using the extracted keypoints.

The exercise posture of the subject U that may be estimated from the exercise image 300 targeted for analysis using the keypoints estimated from the posture estimation model 52 may vary. For example, at least one of the artificial intelligence motion analysis unit 122 or 212 and the rule-based motion analysis unit 123 or 213 may estimate and analyze information on at least one of i) a position of a joint point, ii) a range of motion of a joint of a joint point, iii) a movement path of a joint point, iv) a connection relationship between joint points, and v) a symmetry relationship of a joint point for the subject U.

In addition, the motion analysis unit 122 or 212 may perform an analysis of at least one of a distance of motion of a joint, a speed (or acceleration) of movement of a joint, a body balance of a subject (corresponding to a patient) included in an exercise image targeted for analysis, a body balance, and a body alignment state (e.g., an axial alignment state of a leg, a spinal alignment state, etc.) from the keypoints or image 300 extracted from the exercise image 300 targeted for analysis.

In the present invention, the posture estimation model 52 may also be configured to include the training unit 51. Further, in contrast, the training unit 51 may include the posture estimation model 52, in which case the posture estimation model 52 may be trained by the training unit 51 to perform a posture estimation function. Accordingly, in the present invention, the function performed by the posture estimation model 52 may be described interchangeably as being performed by the training unit 51.

Meanwhile, the user terminal 10 or 20 may be configured to perform a posture analysis result service that provides the user terminal 10 or 20 with an exercise motion analysis result (or an exercise motion analysis report) of a user, which is analyzed based on the keypoints extracted and estimated in the posture estimation model 52 (see FIG. 11).

Here, the user terminal 10 or 20 may be at least one of the patient terminal 10, the doctor terminal 20, or a terminal of a third party.

The system 1000 for providing exercise therapy may be configured to perform the communication with the user terminal 10 or 20. In the present invention, it may also be understood that the system 1000 for providing exercise therapy performs performing the communication is accomplished by the communication unit of the system 1000 for providing exercise therapy.

For example, the communication unit of the system 1000 for providing exercise therapy may be configured to perform the communication with the user terminal 10 or 20 using at least one of wireless LAN (WLAN), wireless-fidelity (Wi-Fi), wireless-fidelity (Wi-Fi) direct, digital living network alliance (DLNA), wireless broadband (WiBro), world interoperability for microwave access (WiMAX), high speed downlink packet access (HSDPA), high speed uplink packet access (HSUPA), long term evolution (LTE), long term evolution-advanced (LTE-A), fifth generation mobile telecommunication (5G), Bluetooth™ radio frequency identification (RFID), infrared communication (infrared data association (IrDA)), ultra-wideband (UWB), ZigBee, near field communication (NFC), Wi-Fi direct, or wireless universal serial bus (wireless USB) technologies.

Meanwhile, the user terminal 10 or 20 described in the present invention means an electronic device. For example, the electronic device may include at least one of a smart phone, a cell phone, a tablet PC, a kiosk, a computer, a laptop, a digital broadcasting terminal, a personal digital assistant (PDA), or a portable multimedia player (PMP). Further, the user terminal 10 or 20 may be an electronic device to which a user account is logged in, connected, or registered.

Here, the user account may mean an account registered in the system 1000 for providing exercise therapy according to the present invention. This user account may be understood as a user ID (identification or identification number).

Meanwhile, in the present invention, a process of receiving an exercise image from the user terminal 10 or 20 may be performed. The system 1000 for providing exercise therapy may receive, in communication with the user terminal 10 or 20, the exercise image 300, in which a user is photographed performing an exercise motion.

In this case, the exercise image 300 that the system 1000 for providing exercise therapy receives from the user terminal 10 or 20 may be understood as an exercise image targeted for an exercise motion analysis of the user.

The system 1000 for providing exercise therapy may receive an exercise image targeted for analysis from the user terminal 10 or 20 at various occasions and paths.

For example, as illustrated in FIG. 11, the system 1000 for providing exercise therapy may, based on a graphical object corresponding to “start an exercise” being selected from the user terminal 10 or 20, control a camera 201 provided on the user terminal 10 or 20 to be in an active state such that the camera 201 photographs an exercise image targeted for analysis. Further, the system 1000 for providing exercise therapy may receive the exercise image targeted for analysis that is photographed by the camera 201 from the user terminal 10 or 20 in real time or based on the completion of the user's exercise.

Next, in the present invention, a process of analyzing an exercise motion related to a specific exercise motion of a user included in an exercise image may proceed based on keypoints extracted from the posture estimation model trained using the training data set including the position information on the joint points.

When receiving the exercise image targeted for analysis from the user terminal 10 or 20, the training unit 51 may, based on the posture estimation model 52 trained using the training target exercise image 300, extract keypoints corresponding to joint points of the user U included in the exercise image targeted for analysis. Further, at least one of the artificial intelligence motion analysis unit 122 or 212 and the rule-based motion analysis unit 123 or 213 may analyze an exercise motion of the subject in the exercise image 300 using the extracted keypoints.

The posture estimation information of the user U estimated by the training unit 51 may include various information. For example, the training unit 51 may estimate i) the position information on the joint points P1 and P2 of the subject U, and ii) the information on the range of motion of the joint of the subject U (angle information).

Next, in the present invention, based on the completion of the analysis above, a process of providing an exercise motion analysis result of the user U related to a specific exercise motion to the user terminal 10 or 20 may proceed.

The system 1000 for providing exercise therapy may process an analysis result of an exercise motion of a user to generate an exercise motion analysis report. Further, the system 1000 for providing exercise therapy may provide the exercise motion analysis report on the user terminal 10 or 20.

For example, as illustrated in FIG. 11, the system 1000 for providing exercise therapy may provide joint point graphic objects corresponding to each of the joint points P1 and P2, at positions corresponding to the joint points P1 and P2 of the user U, in the exercise image of the user. Further, the system 1000 for providing exercise therapy may display joint range of motion information 221 of the specific joint point P1, around the specific joint point P1.

As described above, the system 1000 for providing exercise therapy according to the present invention may perform training for the posture estimation model 52 using the database 40 built based on the training target exercise image 300. Further, the posture estimation model 52 may be used to estimate the exercise posture of the user and perform a service of providing an exercise motion analysis result based on the estimated posture.

The information included in the analysis result may vary. For example, the analysis result may include analysis information on at least one of a range of motion of a joint, a distance of motion, a velocity (or acceleration) of movement of a joint, a body balance of a subject (corresponding to a patient) included in an exercise image targeted for analysis, a body balance, or a body alignment state (e.g., an axial alignment state of a leg, a spinal alignment state, etc.) analyzed from the extracted keypoints or image.

Further, the analysis information may further include a score, which may be an analysis score for an exercise motion (or posture) of the user. The analysis score may be calculated based on a variety of methods (e.g., a rule-based analysis based on a preset standard or an analysis by an artificial intelligence algorithm).

The training data set 400 extracted and generated from the training target exercise image 300 by the training unit 51 may be stored and exist in the database 40.

Hereinafter, the training data set 400 used to estimate an exercise posture of a user will be described in more detail.

As illustrated in FIG. 18A, the training data set 400 may be configured to include data associated with the subject U extracted from the training target exercise image 300.

The training unit 51 may extract data for the subject U from the training target exercise image 300 to configure a training data set 400.

This training data set 400 may be configured as a plurality of sub-data sets 401 to 403. In the present invention, it may be understood that the training data set 400 is a data set corresponding to an upper concept, and the sub-data sets 401 to 403 are data sets corresponding to a lower concept.

The training unit 51 may extract the data for the subject U from each of standard frames 301 to 306 selected based on the preset standard among the plurality of frames constituting the training target exercise image 300, and configure the sub-data sets 401 to 403.

The training unit 51 may select the standard frames 301 to 306 based on various standards. The training target exercise image 300 may be a motion image, or a plurality of still images.

When the training target exercise image 300 is a motion image, the training unit 51 may select the standard frames 301 to 306 based on a predetermined time interval T among the plurality of frames constituting the training target exercise image 300. In another example, the training unit 51 may, when the amount of change in the motion of the subject included in preceding and subsequent frames corresponds to a predetermined amount of change or more, select the preceding and subsequent frames as the standard frames 301 to 306.

The training data included in the training data set according to the present invention may be configured as training data extracted centered on the subject included in the training target exercise image, from each of the standard frames selected based on the preset standard among the plurality of frames constituting the training target exercise image.

Hereinafter, for convenience of description, the “training data set 400” and the “sub-data sets 401 to 403” are not separately distinguished, but the present invention will be described based on the “training data set 400”. The information included in the training data set 400, described below, may be information included in the sub-data sets 401 to 403. In this case, the training data set 400 according to the present invention may be understood to include the plurality of sub-data sets 401 to 403 including information described below.

Meanwhile, as illustrated in (a) of FIG. 18B, the training data set 400 may be configured with a plurality of data groups 410 to 460, each corresponding to a different plurality of information attributes.

The training unit 51 may extract data corresponding to each of the plurality of information attributes from the training target exercise image, and classify (or match) data having the same information attributes of the extracted data into the same data group to generate the training data set 400.

Here, a plurality of information attributes 410a to 450a may be understood as a standard for distinguishing a type of information required for estimating the exercise posture of the subject U from the training target exercise image 300. As illustrated in (b) of FIG. 18B, in the present invention, the plurality of different information attributes (a first information attribute to a fifth information attribute, 410a to 450a) may exist to be predefined.

The training unit 51 may extract training data corresponding to each of the plurality of information attributes 410a to 450a from the training target exercise image, and classify the training data corresponding to the same information attribute into the same data group to generate the training data set 400.

Further, the training unit 51 may, based on an association between the plurality of information attributes 410a to 450a, specify an association for each group between the plurality of data groups 410 to 460, and perform training on the training data set 400 and the association for each group.

Hereinafter, the plurality of data groups and the association for each group will be described in detail.

As illustrated in FIG. 18C, the first data group 410 of the plurality of data groups 410 to 450 may include position information 411 and 412 for the joint points P1 and P2 of the subject U included in the exercise image 300.

As illustrated in (a) of FIG. 18C, in the present invention, the joint points P1 and P2 may mean an area of the subject U corresponding to a joint of the user in the training target exercise image 300. Further, as illustrated in (b) of FIG. 18C, the position information 411 and 412 for joint points may be understood as a position of an area where the joint points P1 and P2 are positioned in the training target exercise image 300.

Meanwhile, the human body is made up of more than 200 bones, and a joint is apart where bone connects to each other, and the human body may consist of a plurality of joints.

In the training unit 51, the joint points of the plurality of joint points of the subject U, which are training targets, may exist to be predefined. That is, the “training target joint point” described in the present invention may be understood as a joint point predefined for training in the present invention among a plurality of joint points of the user.

As illustrated in FIG. 18D, in the database 40, a training target joint point that is a training target of the posture estimation model among the plurality of joint points may be predesignated and exist as reference information 500. Further, there may be a predefined sequence of the plurality of training target joint points in the reference information 500.

A first training target joint point may be defined as a center of head. More specifically, the first training target joint point may be understood as a point that is inferred as (predicted as or corresponds to) a cervical 1 level.

A second training target joint point may be defined as a center of neck. More specifically, the second training target joint point is C3 to C4 level, which is a center of the neck lordotic curve, and may be understood as a midpoint in a middle level of levels 1 and 3 from the front.

A third training target joint point may be defined as a lower end of neck. More specifically, the third training target joint point is C7 to T1 level, which may be understood as a midpoint of a line connecting both clavicle levels.

A fourth training target joint point may be defined as a center of shoulder. More specifically, the fourth training target joint point is a center of humerus head, which may be understood as a position that corresponds to a center of rotation of a continuous motion in a rotational motion in which the arm is abducted to a position that is a central axis of a shoulder joint rotation exercise. In an image that is not a continuous motion of the rotation exercise, a point corresponding to predicted position information of a center of shoulder may correspond to the fourth training target joint point. Further, the fourth training target joint point may exist at each of a center of left shoulder and a center of right shoulder.

A fifth training target joint point may be defined as a center of elbow. More specifically, the fifth training target joint point is a part corresponding to a center of the humerus medial-lateral epicondyle, which may be understood as a midpoint at an elbow level. The fifth training target joint point may exist at each of a center of left elbow and a center of right elbow.

A sixth training target joint point may be defined as a center of wrist. More specifically, the sixth training target joint point is a center of the radius-ulnar styloid process, which may be understood as a midpoint at a wrist level. The sixth training target joint point may exist at each of a center of left wrist and a center of right wrist.

A seventh training target joint point may be defined as a center of hand. More specifically, the seventh training target joint point may be understood as a point corresponding to the 3rd metacarpal head, and may exist at each of a center of left hand and a center of right hand.

An eighth training target joint point may be defined as a center of hip joint (a center of femoral head). More specifically, the eighth training target joint point is a position that is a central axis of a hip joint rotation exercise, which may be understood as a position corresponding to a rotational center of a continuous motion in which the leg is abducted. In an image that is not a continuous motion of the rotation exercise, a point corresponding to predicted position information of a center of hip joint may be understood as the eighth training target joint point. The eighth training target joint point may exist at each of a center of left hip joint and a center of right hip joint.

A ninth training target joint point may be understood as a center of knee. More specifically, the ninth training target joint point is a center of the femur medial-lateral epicondyle, which may be understood as a midpoint at a knee level. The ninth training target joint point may exist at each of a center of left knee and a center of right knee.

A tenth training target joint point may be defined as a center of ankle. More specifically, the tenth training target joint point is a center of the medial-lateral malleolus, which may be understood as a midpoint at an ankle level. The tenth training target joint point may exist at each of a center of left ankle and a center of right ankle.

An eleventh training target joint point may be defined as a center of foot. More specifically, the eleventh training target joint point is a point corresponding to the second metatarsal head, which may exist at each of a center of left foot and a center of right foot.

A twelfth training target joint point may be defined as a center of heel. More specifically, the twelfth training target joint point is a level at which the heel touches the floor, which may exist at each of left heel and right heel. The twelfth training target joint point may not be visible in the image when the subject U is standing perfectly front facing, but may be visible when the foot is even slightly deviated.

A thirteenth training target joint point may be defined as sup. end of lordosis. More specifically, the thirteenth training target joint point is a zypoid of sternum level, which is approximately 8-10T spine level, which may be understood as a midpoint at a middle level between an average level of both sides at level 4 and an average level of both sides at level 8.

A fourteenth training target joint point may be defined as a center of lordosis. More specifically, the fourteenth training target joint point is approximately L2-4 spine level, which may be understood as a midpoint at a middle level between level 13 and an average level of both sides at level 8.

A fifteenth training target joint point may be defined as sup. end of lordosis. More specifically, the fifteenth training target joint point is approximately S1-2 spine level, which may be understood as a midpoint at a middle level between level 14 and an average level of both sides at level 8.

Meanwhile, the first training target joint point P1 may be defined as the center of head 510, and the second training target joint point P2 may exist to be predefined as the center of neck 520. Further, a first sequence, which is the most prioritized sequence, may be defined at the first training target joint point P1, and a second sequence, which is prioritized lower than the first sequence, may be defined at the second training target joint point P2.

In this case, the sequence of training target joint points corresponding to each of the left and right sides of the subject U may be such that the training target joint point corresponding to a first side of the body (e.g., left) is prioritized over the training target joint point corresponding to a second side of the body (e.g., right). For example, a training target joint point P3 corresponding to a center of left wrist 530 may be matched with a sequence defined to be prioritized over a training target joint point P4 corresponding to a center of right shoulder 540.

The training unit 51 may extract coordinate information as the position information 411 and 412 of each of the plurality of predesignated training target joint points P1 and P2 from the training target exercise image 300.

The coordinate information may include at least one of two- or three-dimensional coordinates. When two-dimensional coordinate information is extracted, the training unit 120 may extract the x, y-axis coordinate information of each of the plurality of training target joint points P1 and P2 from the training target exercise image 300. In contrast, when two-dimensional coordinate information is extracted, the training unit 120 may extract the x, y, z-axis coordinate information of each of the plurality of training target joint points P1 and P2 from the training target exercise image 300.

The coordinate information may be extracted in a variety of ways. In particular, the coordinate information of the z-axis may be extracted from a camera (e.g., RGB camera) or various types of sensors (e.g., a distance measurement sensor). Further, the coordinate information of the z-axis may be extracted from the training target image 300 through various artificial intelligence algorithms. When the coordinate information of the z-axis is extracted by an artificial intelligence algorithm, it may be stated that the coordinate information of the z-axis is “estimated” or “predicted”.

Meanwhile, the training unit 51 may, based on the position information 411 and 412 of each of the plurality of training target joint points P1 and P2 corresponding to the first information attribute 410a, classify the position information 411 and 412 into the first data group 410 to generate the first data group 410 and the training data set 400 including the first data group 410.

Taking the extraction of two-dimensional coordinate information (x, y coordination information) as an example, the training unit 121 may extract the position information 411, 412 of each of the plurality of training target joint points P1, P2 in the form of paired x-axis, y-axis coordinate information. The training unit 51 may extract the position information “436]” of the first training target joint point P1 and extract the position information “436]” of the second training target joint point P2. Further, the training unit 51 may generate the training data set 400 configured with the first data group 410 including “436]” and “436]”.

The training unit 51 may, based on the position information 411 and 412 of the training target joint points P1 and P2 constituting the first data group 410, perform training to estimate the positions of the joint points P1 and P2 of the subject U included in the training target exercise image 300.

Meanwhile, as shown in (b) of FIG. 18C, the training unit 51 may, based on a predefined sequence between the plurality of training target joint points P1 and P2, sequentially arrange the position information 411 and 412 of each of the plurality of training target joint points P1 and P2 in the first data group 410 to configure (generate) the training data set 400.

As described above, there may be a predefined sequence of the plurality of training target joint points P1 and P2 in the database 40.

The training unit 51 may sequentially dispose the position information 411 and 412 of the plurality of training target joint points P1 and P2 in the first data group 410 according to a sequence corresponding to the training target joint points P1 and P2, with reference to the database 40, to generate the training data set 400. Further, the training data set 400 may be stored in the database 40 to build the database 40 for the posture estimation.

Specifically, as illustrated in (b) of FIG. 18C, the training unit 51 may arrange, in the first data group 410, the first position information 411 on the first training target joint point P1 corresponding to a first sequence in priority, and, following the first position information 411, the second position information 412 on the first training target joint point P2 corresponding to a second sequence.

Meanwhile, the training unit 51 may, based on whether the training target joint points P1 and P2 are visible in the exercise image 300, extract (or specify) the position information 411 and 412 of the training target joint points P1 and P2 according to a process of one of a first process and a second process.

In the present invention, whether the training target joint point is visible may be understood to mean whether the training target joint points P1 and P2 are visible in the training target exercise image 300.

In the present invention, a visible joint point in the training target exercise image may be referred to as a “training target visible joint point” and an invisible joint point in the training target exercise image may be referred to as a “training target invisible joint point”.

The training unit 51 may judge that the training target joint point is visible in the training target exercise image 300 when the training target exercise image 300 includes a body part of the subject U corresponding to the training target joint points P1 and P2.

The training unit 51 may, based on the training target joint points P1 and P2 being visible in the training target exercise image 300, extract position information on an actual position where the training target joint points P1 and P2 are positioned from the training target exercise image 300 according to the first process.

In the present invention, the position information on the training target joint points P1 and P2 extracted according to the first process may be described to be referred to as “first type of information (first type of position information)” or “substantial position information”.

In contrast, the training unit 51 may judge that the training target joint points P1 and P2 are invisible in the exercise image 300 when the training target exercise image 300 does not include a body part of the subject U corresponding to the training target joint points P1 and P2.

The training unit 51 may, based on the training target joint points P1 and P2 being invisible in the exercise image 300, predict an expected position of the training target joint points P1 and P2 according to the second process, and extract (or specify) predicted position information.

In the present invention, the position information on the training target joint points P1 and P2 extracted according to the second process may be described to be referred to as “second type of information (second type of position information)” or “predicted position information”.

As such, in the present invention, the plurality of position information 411 and 412 included in the first data group 410 may have different extraction processes and type information defined depending on whether the plurality of training target joint points P1 and P2 are visible in the training target exercise image 300.

Meanwhile, the second process may include various data processing to extract (specify) the predicted position information on the invisible training target joint points P1 and P2 in the exercise image 300.

For example, the training unit 51 that extracts the predicted position information according to the second process may, based on an actual position information on the training target joint point visible in the exercise image 300, predict the predicted position information of the training target joint points P1 and P2 that are invisible in the exercise image 300.

In this case, the training unit 51 may assign a weight based on the association with the training target joint points P1 and P2 that are invisible in the exercise image 300 to the plurality of training target joint points P1 and P2 that are visible in the exercise image 300 to specify the predicted position information.

For example, the association between the training target joint points may be set to be higher as the sequence corresponding to the training target joint points P1 and P2 is closer. The association with the training target joint point corresponding to a third sequence may be set such that the training target joint point corresponding to the second sequence is higher than the training target joint point corresponding to the first sequence.

In another example, the association between the training target joint points P1 and P2 may be set to be the highest between the training target joint points P1 and P2 that exist corresponding to the left and right sides of the subject U, respectively. For example, the association with the training target joint point P3 corresponding to the center of left wrist may be set to be highest for the training target joint point P3 corresponding to the center of right wrist (see (a) of FIG. 18C).

Further, the training unit 51 may, based on the motion information of the exercise motion performed by the subject U in the exercise image 300, extract the predicted position information of the training target joint points P1 and P2 that are invisible in the exercise image 300.

In the database 40, motion information including a movement path (e.g., movement position, movement direction) of the body (or joint point) according to the exercise motion may exist to be stored.

The training unit 51 may, with reference to the position information of the training target joint points P1 and P2 visible in the exercise image 300 and the motion information in the database 40, specify the predicted position information of the training target joints that are invisible in the exercise image 300.

Meanwhile, as illustrated in FIG. 18C, the second data group 420 of the plurality of data groups 410 to 450 may be configured with data values 421 and 422 representing whether the training target joint points P1 and P2 of the subject U included in the exercise image 300 are visible.

As illustrated in (b) of FIG. 18C, the data values of the data 421 and 422 included in the second data group 420 may be configured as one of a first data value (e.g., “1”) and a second data value (e.g., “2”) in response to whether the training target joint points P1 and P2 are visible.

The data having the first data value (e.g., “1”) is data representing that the training target joint points P1, P2 are visible in the exercise image 300, which may be understood as information representing that the position information included in the first data group 410 is of a first type (actual position information).

The training unit 51 may extract the first type of position information (actual position information) of the training target joint point when the training target joint points P1 and P2 are visible in the exercise image 300. The training unit 51 may, based on the first type of position information (actual position information) extracted from the exercise image 300, generate (configure) the training data set 400 by including data having the first data value (e.g., “ ”) in the second data group 420.

In contrast, the second data value (e.g., “2”) is data representing that the training target joint points P1, P2 are invisible in the exercise image 300, which may be understood as information representing that the position information included in the first data group 410 is of a second type (predicted position information).

The training unit 51 may extract (or specify) the second type of position information (actual position information) of the training target joint point when the training target joint point is invisible in the exercise image 300. The training unit 51 may, based on the second type of position information (predicted position information) extracted (or specified) from the exercise image 300, generate (configure) the training data set 400 by including the data having the second data value (e.g., “2”) in the second data group 420.

Meanwhile, as illustrated in (b) of FIG. 18C, the training unit 51 may generate (configure) the training data set 400 by arranging the data (or data values, 421 and 422) included in the second data group 420 in the same sequence as the predefined sequence in which the position information 411 and 412 of each of the plurality of training target joint points P1 and P2 are arranged to represent whether each of the plurality of training target joint points is visible within the second data group 420.

The training unit 51 may, based on the predefined sequence between the plurality of training target joint points P1 and P2, sequentially arrange the data 421 and 422 representing whether each of the plurality of training target joint points is visible within the second data group 420.

For example, as illustrated in (b) of FIG. 18C, the training unit 51 may, based on the first training target joint point P1 being visible in the exercise image 300, arrange the data 421 having the first data value (e.g., “ ”) in the second data group 420 in the first sequence corresponding to the first training target joint point P1.

Further, although the second training target joint point P2 is illustrated as being visible in the exercise image 300 in (a) of FIG. 18C, assume that the second training target joint point P2 is invisible in the exercise image 300. The training unit 51 may, based on the second training target joint point P2 being visible in the exercise image 300, arrange the data 422 having the second data value (e.g., “2”) in the second sequence corresponding to the second training target joint point P2.

Meanwhile, in the present invention, it may be understood that the definition of the type of position information included in the first data group 410 is made by the data value that the data included in the second data group 420 has.

As illustrated in (b) of FIG. 18C, assume that in the second data group 420, the data 421 arranged in the first sequence has the first data value (e.g., “ ”) and the data 422 arranged in the second sequence has the second data value (e.g., “2”).

In the present invention, a type of the position information 411 arranged in the first sequence within the first data group 410 may be defined as the first type of position information (actual position information) based on the data 421 arranged in the first sequence within the second data group 420 having the first data value (e.g., “1”).

In contrast, the type of the position information 411 arranged in the second sequence within the first data group 410 may be defined as the second type of position information (predicted position information) based on the data 422 arranged in the second sequence within the second data group 420 having the second data value (e.g., “2”).

Meanwhile, the posture estimation model 52 according to the present invention may perform training by setting different training weights for the position information 411 and 412 of each of the plurality of training target joint points included in the first data group 410 based on the data value included in the second data group 420.

Specifically, when the data 421 arranged in the first sequence within the second data group 420 has the first data value (e.g., “1”), the posture estimation model 52 may perform training by setting a first training weight for the position information 411 arranged in the first sequence within the first data group 410.

In contrast, when the data 422 arranged in the second sequence within the second data group 420 has the second data value (e.g., “2”), the posture estimation model 52 may perform training by setting a second training weight for the position information 412 arranged in the second sequence within the first data group 410.

That is, the posture estimation model 52 may perform training by setting different training weights for the first type of position information (actual position information) and the second type of position information (predicted position information) based on the data value of the second data group 420.

In this case, the posture estimation model 52 may set the first training weight to be higher than the second training weight.

Meanwhile, as illustrated in FIG. 18B, the plurality of data groups 410 to 450 may further include a third data group 430 that includes information related to a photographing direction for the subject included in the exercise image 300. The third data group 430 may be configured with a data value representing a photographing direction in which the subject U included in the exercise image 300 is photographed.

As illustrated in (a) of FIG. 18E, the subject U may be photographed from different photographing directions (e.g., “front” or “side”).

Here, the term “photographing direction” may be understood as a direction of an axis of the camera (see reference numeral “201” in FIG. 11) with respect to the subject U. Here, the camera 201 may be understood as the camera 201 that photographs the exercise image 300 including the subject U. The camera 201 may include the camera 201 provided on the user terminal 10 or 20.

The data values included in the third data group 430 illustrated in (b) of FIG. 18E may have different data values (e.g., “0” or “1”) in response to the photographing direction for the subject U. The data values included in the third data group may be configured to have different data values depending on the photographing direction of the subject, with respect to the camera that photographs the subject. Hereinafter, in order to avoid terminological confusion with the data value included in the second data group 420, the data value corresponding to the photographing direction will be described to be referred to as a “data object value”.

The data having a first data object value (e.g., “0”) may be understood as data in which the photographing direction for the training target exercise image 300 represents a first direction (e.g., a frontal direction) (see (b) of FIG. 18E).

The training unit 51 may, based on the photographing direction for the subject U included in the training target exercise image 300 corresponding to the first direction (e.g., frontal direction), generate the training data set 400 by including data having the first data object value (e.g., “0”) in the third data group 430.

In contrast, a second data object value (e.g., “1”) may be understood as data in which the photographing direction for the exercise image 300 is a second direction (e.g., a lateral direction) that is different from the first direction (see (b) of FIG. 18E).

The training unit 51 may, based on the photographing direction for the subject U included in the exercise image 300 corresponding to the second direction (e.g., lateral direction), generate the training data set 400 by including data having the second data object value (e.g., “1”) in the third data group 430.

Further, although not illustrated, a third data object value (e.g., “2”) may be understood as data in which the photographing direction for the exercise image 300 represents a third direction (e.g., an oblique direction) that is different from the first and second directions.

The training unit 51 may, based on the photographing direction for the subject U included in the training target exercise image 300 corresponding to the third direction (e.g., an oblique direction), allow data having the third data object value (e.g., “2”) to be included in the third data group 430 to be stored in the database 40.

Meanwhile, the first direction to the third direction described in the present invention may be understood as a case where an angle formed by the axis of the camera 201 and the subject U, with respect to a preset direction (e.g., clockwise direction), corresponds to each of a first range to a third range.

For example, the first direction may be understood that the angle formed by the axis of the camera 201 and the subject U corresponds to a range between a first angle and a second angle greater than the first angle. The second direction may be understood that the angle formed by the axis of the camera 201 and the subject U corresponds to a range between the second angle and a third angle greater than the second angle. Further, the third direction may be understood that the angle formed by the axis of the camera 201 and the subject U corresponds to a range between the second angle and a third angle greater than the second angle.

Meanwhile, the training unit 51 may configure the angle (or angle value) formed by the subject U and the axis of the camera 201 with respect to the preset direction (e.g., clockwise direction) as the data value of the data included in the third data group 430. For example, when the subject U and the axis of the camera 201 are perpendicular with respect to the clockwise direction, the training unit 51 may configure the data value of “90” as the data of the third data group 430. Meanwhile, the training unit 51 may perform training for estimating the posture of the subject U in conjunction with the training data set 400, which has different photographing direction information included in the third data group 430.

For example, assume that there are a first training data set with a first data object value (e.g., “0”) and a second training data set with a second data object value (e.g., “1”) in the data included in the third data group 430. The training unit 51 may perform training for estimating the posture of the subject U in conjunction together with the position information of the first data group 410 included in the first data set and the position information of the first data group included in the second data set.

Meanwhile, when estimating the exercise posture of the subject U from an exercise image targeted for analysis photographed in the first direction, the training unit 51 may estimate the exercise posture of the subject U based on the posture estimation model performed training on the training target exercise image 300 photographed in the training target exercise image 300 photographed in the first direction.

The system 1000 for providing exercise therapy according to the present invention may analyze the exercise motion of the subject U based on the keypoints extracted from the posture estimation model trained using the training data set 400, which includes the photographing direction information (data object value or data value) corresponding to the photographing direction of the exercise image targeted for analysis.

Meanwhile, when estimating the exercise posture of the subject U from the exercise image targeted for analysis 300 photographed in the first direction, the motion analysis unit 120 or 210 may analyze the exercise motion of the subject U using the posture estimation information estimated from the posture estimation model trained on the training target exercise image 300 photographed in each of the first direction and the second direction.

That is, when the exercise motion of the subject U is photographed from the exercise image targeted for analysis 300 photographed in a first photographing direction, the motion analysis unit 120 or 210 may analyze the exercise motion of the subject U using the posture estimation information estimated from the posture estimation model that has performed training on the first training data set including data object values corresponding to the first photographing direction and the second training data set including data object values corresponding to a second photographing direction different from the first photographing direction. In this case, the posture estimation model 122 may correspond to a posture estimation model that has been performed training by setting a weight on the first training data set.

As such, the posture estimation model 52 may, according to the photographing direction of the subject included in the training target image, be trained through the training data set having different data values in consideration of the photographing direction of the subject. Further, the exercise motion analysis result of the user may be a result of analyzing a specific exercise motion of the user based on the posture estimation information extracted in consideration of the photographing direction of the user included in the exercise image in the posture estimation model.

Meanwhile, as illustrated in FIG. 18B, a fourth data group 440 of the plurality of data groups may include an exercise code matched to an exercise motion performed by the subject U included in the training target exercise image 300.

As illustrated in FIG. 18F, in the database 40, there may be different exercise codes (e.g., “502”, “503”, “504”) matched to each of the different plurality of exercise motions 710, 720, and 730.

The term “exercise code” described in the present invention is a data value that distinguishes different exercise motions, which may be used interchangeably with the terms “exercise key,” “motion code,” and “motion key.”

The training unit 51 may generate the training data set 400 by including, in the fourth data group 440, a specific exercise code (“502”) matched to a specific exercise motion (e.g., “stand on one foot and extend the other foot forward”, 710) performed by the subject U included in the training target exercise image 300.

The training unit 51 may associate the plurality of training data sets 400 including the same exercise codes with each other to perform training for the posture estimation.

For example, assume that there is a first training data set based on a first training target exercise image, and a second training data set based on a second training target exercise image 300. The training unit 51 may, based on the exercise codes (e.g., “502”) included in the first training data set and the second training data set being the same, associate the first training data set and the second training data set with each other to perform training for the posture estimation.

These exercise codes may be included in the fourth data group 440, based on the exercise motion performed by the subject U being specified by at least one of the information received from the user terminal 10 or 20, the system administrator, or the training unit 51.

The training unit 51 may, based on the information received from the user terminals 10 or 20, specify the exercise motion performed by the subject U. For example, the training unit 51 may, based on a graphical object corresponding to “start an exercise” being selected from the user terminal 10 or 20, control a camera 201 provided on the user terminal 10 or 20 to be in an active state such that the camera 201 photographs an exercise image.

In this case, the graphic object may correspond to a specific exercise motion, and the training unit 51 may judge that the subject U included in the exercise image 300 received from the user terminal 10 or 20 has performed the specific exercise motion.

Further, the training unit 51 may, based on information input by the system administrator, specify the exercise motion performed by the subject U.

Further, the training unit 51 may, based on the position information of the training target joint of the subject U included in the exercise image 300, specify the exercise motion performed by the subject U. In this case, with reference to the motion information for the exercise motion stored in the database 40, the exercise motion performed by the subject U may be specified.

Meanwhile, the training unit 51 may match the plurality of training data sets 400 including the same exercise code to each other, on the basis of the exercise code included in the fourth data group 440, to be stored in the database 40.

In this case, the training unit 51 may, on the basis of the training code, divide the memory (or memory space) of the database 40 to be allocated. In the present invention, the dividing of the memory (or memory space) of the database 40 may be understood as generating a folder on the database 40 on the basis of the exercise code. Further, the training unit 51 may store the training data set 400 including a specific exercise code in a folder corresponding to the specific exercise code.

Meanwhile, the motion analysis unit 120 or 210 may analyze an exercise motion using the posture estimation information estimated from the posture estimation model trained using a training data set including the exercise code corresponding to the specific exercise motion performed by the subject U in the exercise image targeted for analysis.

Further, the motion analysis units 120 and 210 may estimate the exercise motion of the subject U for the specific exercise motion using the posture estimation information estimated from the posture estimation model trained based on the training target exercise image 300 related to the same specific exercise motion as the subject U included in the analysis exercise target.

Meanwhile, as illustrated in FIG. 18C, a fifth data group 450 of the plurality of data groups 410 to 450 may include size information 451 on the bounding box 301 for the subject U included in the training target exercise image 300, and position information 452 centered on the bounding box 301.

In the present invention, the term “size information” of the bounding box 301 may be used interchangeably with the term “scale”.

The training unit 51 may extract the size information 451 on the bounding box 301 corresponding to the subject U detected in the training target exercise image 300, extract center position information 452 on the bounding box 301, and generate the training data set 400 by including the extracted size and center position information in the fifth data group 450.

As described above, the training unit 51 may use an algorithm for various object detections to detect the subject U from the training target exercise image 300. For example, the training unit 51 may use an algorithm (weighted box fusion (WBF)) that ensembles a plurality of bounding boxes. However, it is obvious that the training unit 51 is not limited to the object detection algorithm described above, but may utilize various object detection algorithms that are capable of detecting an object corresponding to the subject U from the training target exercise image 300.

The training unit 51 may, based on the object detection algorithm, extract the size information 451 and the center position information 452 on the bounding box corresponding to the subject U from the training target image 300, and include the size information 451 and the center position information 452 in the fifth data group 450 to generate the training data set 400.

In this case, the training unit 51 may extract the center position information 452 on the bounding box 301 in the form of paired x-axis and y-axis coordinate information.

Meanwhile, the training unit 51 may configure the training data set 400 by including image identification information on the training target exercise image 300.

Here, the term “image identification information” refers to information for identifying the image 300 from which the information included in the training data set is extracted, which may include, for example, filename information, file format type information (or extension information, e.g., “JPG”, “TIF”) of the training target exercise image 300.

In the present invention, the image identification information may be referred to as a sixth data group corresponding to a sixth information attribute.

The training unit 51 may generate the training data set 400 by including the sixth data group configured with the image identification information.

Meanwhile, in the present invention, the exercise motion analysis result may be provided to the user terminal 200 on the basis of the completion of an analysis of the exercise motion of the user included in the exercise image targeted for analysis through the posture estimation information extracted based on the posture estimation model 122.

For example, as illustrated in (a) of FIG. 19, the service server 130 may, based on a graphical object 210 corresponding to “start an exercise” being selected from the user terminal 200, control a camera 201 provided on the user terminal 200 to be in an active state such that the camera 201 photographs an exercise image targeted for analysis. Further, the service server 130 may receive the exercise image targeted for analysis that is photographed by the camera 201 from the user terminal 200 in real time or based on the completion of the user's exercise.

As illustrated in (b) of FIG. 19, the service server 130 may provide the exercise motion analysis result using the analysis result to the user terminal 200 on the basis of the completion of the analysis of the exercise motion of the user included in the image to be analyzed based on the posture estimation model 122. The exercise motion analysis result provided to the user terminal 200 by the service server 130 may include a variety of information. For example, the service server 130 may render the graphic objects corresponding to the joint points P1 and P2 to the subject U corresponding to the user and provide them on the user terminal 200. In this case, the service server 130 may provide the joint range of motion information (angle information, 221) of the subject U together.

Further, as illustrated in (c) of FIG. 19, the service server 130 may provide, on the user terminal 200, a plurality of exercise posture information 820 for each joint point of the subject U performing a specific exercise posture (e.g., “arms out to side”, 810). For example, the service server 130 may provide, on the user terminal 200, a graph of a range of motion of a joint for a joint point positioned on a first side (e.g., left) of the user and a range of motion of a joint for a joint point positioned on a second side (e.g., right) of the patient.

Meanwhile, the service server 130 may, based on a motion analysis result for a specific exercise motion of the user, prescribe (provide) an exercise plan (or exercise program) including at least one exercise motion to the user.

For example, as illustrated in (a) of FIG. 20, the service server 130 may, based on the exercise motion analysis result, add an exercise motion to an exercise plan (or exercise program) and provide, on the user terminal 200, a service page 910 that includes information on the exercise plan (or exercise program) including the added exercise motion.

In another example, as illustrated in (b) of FIG. 20, the service server 130 may, based on the motion analysis posture result, adjust the difficulty level of an exercise plan (or exercise program) and provide, on the user terminal 200, a service page 920 that includes information on the adjusted difficulty level.

In yet another example, as illustrated in (c) of FIG. 20, the service server 130 may, based on the exercise motion analysis result, exclude a portion of an exercise motion from an exercise plan (or exercise program) and provide, on the user terminal 200, a service page 930 guiding that the exercise motion has been excluded.

Meanwhile, in the present invention, an exercise motion analysis result may be provided to the patient terminal 10 on the basis of completing an analysis of the exercise motion of the patient based on the motion analysis unit 120 or 210 as described above. As illustrated in FIGS. 21A, 21B, and 21C, the exercise motion analysis results may be provided through the exercise therapy application 100 installed on the patient terminal 10.

As illustrated in (a) of FIG. 21A, the exercise therapy application 100 may provide, on the patient terminal 10, a service page configured to be accessible for each of a plurality of services provided in the present invention. For example, the service page may be configured to be accessible to at least one of i) an exercise guide page associated with a function of providing exercise guide information for an exercise plan allocated to the patient account, ii) an exercise page related to performing an exercise plan allocated to the patient account (see (b) of FIG. 21A), iii) a functional evaluation page associated with a functional evaluation (see (c) of FIG. 21A), or iv) a plan evaluation page associated with an exercise plan evaluation function.

Further, as illustrated in FIG. 21B, the exercise therapy application 100 may provide, on the patient terminal 10, an exercise report page that provides an exercise report based on an exercise motion analysis result and an exercise performance result. For example, the exercise report page may include at least one of exercise performance rate information (see (a) and (b) of FIG. 21B) or exercise plan difficulty information (see (c) of FIG. 21B).

Further, as illustrated in FIG. 21C, the exercise therapy application 100 may provide, on the patient terminal 10, the exercise motion analysis result of the patient.

As illustrated in (a) of FIG. 21C, the exercise therapy application 100 may provide, on the patient terminal 10, a plurality of exercise motion analysis information for each joint point of the subject U performing a specific prescribed exercise (e.g., “arms out to side”). For example, the exercise therapy application 100 may provide, on the patient terminal 10, a graph of a range of motion of a joint for a joint point positioned on a first side (e.g., left) of the patient and a range of motion of a joint for a joint point positioned on a second side (e.g., right) of the patient.

As illustrated in (b) of FIG. 21C, the exercise therapy application 100 may provide an exercise motion analysis result of the patient who performed a prescribed exercise according to an exercise plan over a period of time, on a daily basis. For example, the exercise therapy application 100 may provide joint range of motion information corresponding to a first exercise day and joint range of motion information corresponding to a second exercise day. Further, the exercise therapy application 100 may provide an average range of motion of a joint on the first exercise day and the second exercise day.

As illustrated in (c) of FIG. 21C, the exercise therapy application 100 may render graphic objects corresponding to the keypoints P1 and P2 on the exercise image 300 and provide the graphic objects on the patient terminal 10. In this case, the exercise therapy application 100 may provide the joint range of motion information (angle information) of the patient together.

Meanwhile, the system 1000 for providing exercise therapy according to the present invention may also provide the exercise motion analysis result provided to the patient terminal 10 to the doctor terminal 20 such that the doctor may perform monitoring on the performance of the exercise plan of the patient.

As described above, the method and system for providing exercise therapy using an artificial intelligence posture estimation model and motion analysis model, according to the present invention, can receive, from a doctor terminal, prescription information related to an exercise for a patient, and, based on the prescription information, allocate an exercise plan including at least one prescribed exercise to an account of the patient. This allows a doctor to prescribe to a patient, and a patient to be provided with an exercise plan based on the doctor's prescription, even if the doctor and patient do not meet in person for exercise therapy for a musculoskeletal disease, thereby resolving spatial, temporal, and economic constraints on the exercise therapy and increasing accessibility to the exercise therapy.

Further, the method and system for providing exercise therapy using an artificial intelligence posture estimation model and motion analysis model, according to the present invention, can analyze an exercise motion of a user by extracting a keypoint corresponding to each of a plurality of preset joint points from an exercise image to focus on a joint required for exercise therapy of a musculoskeletal disease.

Further, the method and system for providing exercise therapy using an artificial intelligence posture estimation model and motion analysis model, according to the present invention, can analyze an exercise motion related to a specific exercise motion of a user included in an exercise image based on a posture estimation model trained using a training data set including position information for a joint point. Therefore, in the present invention, it is possible to accurately analyze posture of a patient from an exercise image, and in particular, it is possible to improve the quality of healthcare services by obtaining information on a range of motion, alignment state, and deviation state of a joint of the patient.

Further, the method and system for providing exercise therapy using an artificial intelligence posture estimation model and motion analysis model, according to the present invention, by transmitting an analysis result of an exercise motion of a patient to a patient terminal, the patient can be provided with feedback on an exercise image without having to visit a hospital located at a distance, thereby enhancing an effect of exercise therapy.

Meanwhile, the present invention described above may be executed by one or more processes on a computer and implemented as a program that can be stored on a computer-readable medium (or recording medium).

Further, the present invention described above may be implemented as computer-readable code or instructions on a medium in which a program is recorded. That is, the present invention may be provided in the form of a program.

Meanwhile, the computer-readable medium includes all kinds of storage devices for storing data readable by a computer system. Examples of computer-readable media include hard disk drives (HDDs), solid state disks (SSDs), silicon disk drives (SDDs), ROMs, RAMs, CD-ROMs, magnetic tapes, floppy discs, and optical data storage devices.

Further, the computer-readable medium may be a server or cloud storage that includes storage and that the electronic device is accessible through communication. In this case, the computer may download the program according to the present invention from the server or cloud storage, through wired or wireless communication.

Further, in the present invention, the computer described above is an electronic device equipped with a processor, that is, a central processing unit (CPU), and is not particularly limited to any type.

Meanwhile, it should be appreciated that the detailed description is interpreted as being illustrative in every sense, not restrictive. The scope of the present invention should be determined based on the reasonable interpretation of the appended claims, and all of the modifications within the equivalent scope of the present invention belong to the scope of the present invention.

What is claimed is:

1. A method of providing digitally-based musculoskeletal rehabilitation therapy provided in an application, the method comprising:
selecting, based on prescription information including an exercise plan corresponding to an indication of a patient being allocated from a doctor terminal, an exercise plan to be provided to the patient;
executing the application on a user terminal to which an account of the patient is logged in;
providing an exercise list according to the exercise plan to the user terminal on which the application is executed;
playing, on the user terminal, an exercise image corresponding to each of a plurality of exercise items according to the plurality of exercise items constituting the exercise list;
providing, based on a degree of playback of the exercise image satisfying a preset standard, an evaluation page to the user terminal to receive evaluation for the exercise plan from the patient; and
updating the exercise plan based on evaluation information received through the evaluation page and exercise matching information present in the storage unit,
wherein the evaluation information comprises at least one of first evaluation information in which the patient evaluates difficulty of the exercise plan, and second evaluation information in which the patient selects at least one exercise item of the plurality of exercise items as an exercise of high difficulty,
wherein the exercise matching information includes, for each indication, exercise items for the indication therapy that are matched as a group, and each of the exercise items for the indication therapy is matched with difficulty level information of the exercise, and
wherein the updating of the exercise plan comprises:
changing, based on the first evaluation information and the exercise matching information, difficulty of the exercise items constituting the exercise plan;
excluding, based on the second evaluation information and the exercise matching information, the exercise item selected as an exercise of high difficulty from the exercise plan; and
allowing a different exercise item to be included in the exercise plan, the different exercise item being matched to the same group of the selected exercise item and being matched with the same difficulty level information of the selected exercise item.

2. The method of claim 1, wherein the exercise plan includes exercise items related to an indication of the patient included in the prescription information on the patient, at least some of which are allocated to each of a plurality of different days constituting a preset rehabilitation period.

3. The method of claim 2, wherein in the providing of the exercise list, the user terminal provides the exercise list including the plurality of exercise items allocated to specific days on which the exercise image is played, based on a reference date from which a counting of the preset rehabilitation period has been started, and
wherein the evaluation page is provided to the user terminal for performing an evaluation related to the plurality of exercise items provided to the patient on each of the specific days, when a degree of playback of the exercise image satisfies a preset standard.

4. The method of claim 3, wherein the evaluation page includes at least one of a first evaluation area configured to evaluate difficulty for the plurality of exercise items allocated to the specific day, a second evaluation area configured to select a high difficulty exercise among the plurality of exercise items, and a third evaluation area configured to evaluate exercise pain related to the plurality of exercise items, and
wherein in the updating of the exercise plan, the difficulty of the exercise items constituting the exercise plan is changed, or at least some of the exercise items constituting the exercise plan are replaced by the different exercise item, based on the evaluation information received through at least one of the first evaluation area, the second evaluation area, and the third evaluation area.

5. The method of claim 4, wherein the exercise items according to the updated exercise plan are provided to the user terminal, beginning on a day after the specific day has elapsed.

6. The method of claim 1, further comprising:
allocating, to the patient account, a cognitive behavioral therapy plan that proceeds in conjunction with the exercise plan,
wherein the allocating of the cognitive behavioral therapy plan comprises:
receiving, through the user terminal, survey response data for a plurality of survey data;
detecting, based on the survey response data, state information on the patient related to pain duration and a degree of cognitive distortion of the patient;
specifying a user group corresponding to the state information on the patient among a plurality of user groups categorized according to the pain duration and degree of cognitive distortion;
determining an initial therapy protocol corresponding to the user group among a plurality of therapy protocols; and
providing a plurality of specific therapy programs included in the initial therapy protocol, sequentially during a preset rehabilitation period.

7. The method of claim 6, further comprising:
providing, in response to the application being executed on the user terminal, an initial screen page,
wherein the initial screen page includes at least one of:
a first menu item configured to access an exercise list according to the exercise plan;
a second menu item configured to access the evaluation page;
a third menu item configured to access the cognitive behavioral therapy plan allocated in conjunction with the exercise plan; and
a fourth menu item configured to access a page for performing a functional evaluation of a specific motion of the patient, and
wherein, when the degree of playback of the exercise image does not satisfy the preset standard, the provision of the evaluation page to the user terminal is restricted, even though the second menu item is selected on the user terminal.

8. The method of claim 7, wherein the functional evaluation of the specific motion is configured such that the exercise plan is performed at a preset day interval during the preset rehabilitation period to which the exercise plan is allocated, and
wherein the fourth menu item is configured to be included in the initial screen page on a specific day according to the preset day interval, and not to be included in the initial screen page, when not on the specific day during the rehabilitation period.

9. A system for providing digitally-based musculoskeletal rehabilitation therapy, the system comprising:
a communication unit configured to receive, from a doctor terminal, prescription information including an exercise plan corresponding to an indication of a patient;
a storage unit configured to store exercise matching information in which, for each indication, exercise items for the indication therapy are matched as a group, and each of the exercise items for the indication therapy is matched with difficulty level information of the exercise; and
a control unit, in response to an execution of an application on a user terminal to which the patient account is logged in, configured to provide an exercise list according to the exercise plan to the user terminal,
wherein the control unit is configured to:
play, on the user terminal, an exercise image corresponding to each of a plurality of exercise items constituting the exercise list according to the plurality of exercise items;
provide, based on a degree of playback of the exercise image satisfying a preset standard, an evaluation page to the user terminal for the patient to perform evaluation for the exercise plan;
update the exercise plan based on evaluation information received through the evaluation page and exercise matching information stored in the storage unit,
wherein the evaluation information includes at least one of first evaluation information in which the patient evaluates difficulty of the exercise plan, and second evaluation information in which the patient selects at least one exercise item of the plurality of exercise items as an exercise of high difficulty,
wherein the control unit is configured to:
change, based on the first evaluation information and the exercise matching information, difficulty of the exercise items constituting the exercise plan;
exclude, based on the second evaluation information and the exercise matching information, the exercise item selected as an exercise of high difficulty from the exercise plan; and
allow a different exercise item to be included in the exercise plan, the different exercise item being matched to the same group of the selected exercise item and being matched with the same difficulty level information of the selected exercise item.

10. A program stored on a computer-readable recording medium, executable by one or more processes on an electronic device, the program comprising instructions for performing of:
selecting, based on prescription information including an exercise plan corresponding to an indication of a patient being allocated from a doctor terminal, an exercise plan to be provided to the patient;
executing the application on a user terminal to which an account of the patient is logged in;
providing an exercise list according to the exercise plan to the user terminal on which the application is executed;
playing, on the user terminal, an exercise image corresponding to each of a plurality of exercise items according to the plurality of exercise items constituting the exercise list;
providing, based on a degree of playback of the exercise image satisfying a preset standard, an evaluation page to the user terminal for the patient to perform evaluation for the exercise plan; and
updating the exercise plan based on evaluation information received through the evaluation page and exercise matching information present in the storage unit,
wherein the evaluation information comprises at least one of first evaluation information in which the patient evaluates difficulty of the exercise plan, and second evaluation information in which the patient selects at least one exercise item of the plurality of exercise items as an exercise of high difficulty,
wherein the exercise matching information includes, for each indication, exercise items for the indication therapy that are matched as a group, and each of the exercise items for the indication therapy is matched with difficulty level information of the exercise,
wherein the updating of the exercise plan comprises:
changing, based on the first evaluation information and the exercise matching information, difficulty of the exercise items constituting the exercise plan;
excluding, based on the second evaluation information and the exercise matching information, the exercise item selected as an exercise of high difficulty from the exercise plan; and
allowing a different exercise item to be included in the exercise plan, the different exercise item being matched to the same group of the selected exercise item and being matched with the same difficulty level information of the selected exercise item.
